(19) United States
(12) Patent Application Publication
(10) Pub. No.: US 2026/0215671 A1
(43) Pub. Date: Jul. 30, 2026

(54) ORAL IMAGING DEVICE

(71) Applicant: GUANGZHOU STARS PULSE CO.,LTD., GUANGZHOU (CN)

(72) Inventors: HANG FAN, GUANGZHOU (CN); LULU NING, GUANGZHOU (CN); HANHONG DAI, GUANGZHOU (CN); HONGQUAN MO, GUANGZHOU (CN); BO CHEN, GUANGZHOU (CN)

(21) Appl. No.: 19/460,470

(22) Filed: Jan. 27, 2026

(30) Foreign Application Priority Data
Jan. 27, 2025 (WO) PCT/CN2025/075430
Jan. 27, 2025 (WO) PCT/CN2025/075539
Apr. 30, 2025 (CN) 202510570684.7

(51) Int. Cl. A61B 1/24; A61B 1/00; A61B 1/05

(52) U.S. Cl. CPC A61B 1/24; A61B 1/00105; A61B 1/05

(57) ABSTRACT

An oral imaging device includes an oral camera and a mouth opener. The oral camera includes a main body and a camera module. The camera module includes at least one camera. The mouth opener defines an imaging through hole penetrating through the mouth opener from a first end to a second end thereof. The second end of the mouth opener is configured to abut against an oral cavity structure of a user. The mouth opener is movable relative to the camera module to switch the oral imaging device between a first form and a second form. In the first form, the at least one camera faces the imaging through hole and does not exceed the second end of the mouth opener. In the second form, the at least one camera is exposed outside the imaging through hole and is configured to insert into an oral cavity of the user.

### TECHNICAL FIELD

The present disclosure relates to a technical field of oral detection, and in particular to an oral imaging device.

### BACKGROUND

Oral cameras generally include in-mouth oral cameras and oral cameras with mouth openers. The in-mouth oral cameras are configured to shoot tooth surfaces in the mouth, which are commonly allowed to shoot a single tooth or several teeth. The in-mouth oral cameras are difficult to shoot a panoramic image of the teeth, and when shooting the teeth, one or more teeth may be missed. Further, when shooting outer surfaces of the teeth, the in-mouth oral cameras are easy to be blocked by the lips, making it difficult to shoot the outer surfaces of all teeth. The oral cameras with mouth openers are configured to shoot teeth outside the mouth, and images of the outer surfaces of the teeth are obtained. However, since occlusal surfaces of the teeth are only shot in a direction perpendicular to the teeth and inner surfaces of the teeth are only shot inside the mouth, it is difficult for oral cameras with the mouth opener to obtain images of the inner side of some teeth.

Currently, in order to obtain better images of the outer surfaces, the occlusal surfaces and the inner surfaces of the teeth, and to obtain a panoramic image of the occlusal surfaces of the teeth that is easy to observe, professionals, such as dentists, use mouth openers combined with professional single-lens reflex (SLR) cameras and reflectors for shooting, which is complicated to use.

Oral shooting schemes in the related art are difficult to balance all-round shooting of teeth and ease of operation.

### SUMMARY

The present disclosure provides an oral imaging device. The oral imaging device has a shooting view angle and realizes comprehensive shooting of occlusal surfaces, inner surfaces and outer surfaces of teeth through shooting outside and inside a mouth.

In order to achieve the above-mentioned purpose, the present disclosure provides an oral imaging device. The oral imaging device includes an oral camera, and a mouth opener. The oral camera includes a main body and a camera module connected to the main body. The camera module includes at least one camera. The mouth opener includes a first end and a second end opposite to the first end. The mouth opener defines an imaging through hole penetrating through the mouth opener from the first end to the second end. The second end of the mouth opener is configured to abut against an oral cavity structure of a user. A position of the mouth opener is variable relative to the at least one camera to enable the oral imaging device to have at least a first form and a second form. In the first form, the at least one camera faces the imaging through hole and does not exceed the second end of the mouth opener. In the second form, the at least one camera is exposed outside the imaging through hole and is configured to insert into an oral cavity of the user.

When the oral imaging device in the embodiment of the present disclosure is in the first form, the mouth opener opens the mouth and supports the lips to expose the outer surfaces of the teeth, so that the outer surfaces of the teeth of the user face the imaging through hole of the mouth opener, and the camera module at least receives the light reflected back from the outer surfaces of the teeth and takes images. In the second form, the camera module is inserted into the oral cavity to take images of the inner surfaces of the teeth and the occlusal surfaces of the teeth. Because the camera module is inserted into the oral cavity, the camera module is able to obtain a good shooting angle. The oral imaging device has the shooting view angle and realizes comprehensive shooting of the occlusal surfaces, the inner surfaces and the outer surfaces of teeth through shooting outside and inside the mouth.

### BRIEF DESCRIPTION OF DRAWINGS

The drawings are included to provide a further understanding of embodiments of the present disclosure, which form portions of the specification and are used to illustrate implementation manners of the present disclosure and are intended to illustrate operating principles of the present disclosure together with the description. Apparently, the drawings in the following description are merely some of the embodiments of the present disclosure, and those skilled in the art are able to obtain other drawings according to the drawings without contributing any inventive labor. In the drawing:

FIG. 1 is a schematic diagram of an oral imaging device according to a first embodiment of the present disclosure.

FIG. 2 is an exploded schematic diagram of the oral imaging device according to the first embodiment of the present disclosure.

FIG. 3 is another exploded schematic diagram of the oral imaging device according to the first embodiment of the present disclosure.

FIG. 4 is a schematic diagram of the oral imaging device according to a second embodiment of the present disclosure.

FIG. 5 is an exploded schematic diagram of the oral imaging device according to the second embodiment of the present disclosure.

FIG. 6 is a schematic diagram of the oral imaging device according to a third embodiment of the present disclosure.

FIG. 7 is a schematic diagram of the oral imaging device shown in FIG. 6, where a mouth opener is slid relative to a main body to stagger a through hole and a camera.

FIG. 8A is a cross-sectional schematic diagram of the oral imaging device taken along a line A-A shown in FIG. 1.

FIG. 8B is a cross-sectional schematic diagram of the oral imaging device taken along the line A-A shown in FIG. 1 where the mouth opener is provided.

FIG. 9 is another cross-sectional schematic diagram of the oral imaging device taken along the line A-A shown in FIG. 1.

FIG. 10 is another cross-sectional schematic diagram of the oral imaging device taken along the line A-A shown in FIG. 1.

FIG. 11 is another cross-sectional schematic diagram of the oral imaging device taken along the line A-A shown in FIG. 1.

FIG. 12 is a schematic diagram of the oral imaging device according to a fourth embodiment of the present disclosure.

FIG. 13 is a schematic diagram of the oral imaging device shown in FIG. 3, where a support is connected to another position of the main body.

FIG. 14 is a schematic diagram of the oral imaging device according to a fifth embodiment of the present disclosure.

FIG. 15 is a front side schematic diagram of the oral imaging device shown in FIG. 14.

FIG. 16 is a schematic diagram of a shooting range of the oral imaging device, where a mouth opening angle of a user is β and an inclined angle of the camera is α.

FIG. 17 is a schematic diagram of a field of view (FOV) of the camera.

FIG. 18 is a schematic diagram of one of dental arches.

FIG. 19 is a schematic diagram of commonly oral images in clinical practice.

FIG. 20 is a schematic diagram of a first embodiment of an oral camera of the present disclosure.

FIG. 21 is a side schematic diagram of the oral camera shown in FIG. 20.

FIG. 22 is another side schematic diagram of the oral camera shown in FIG. 20.

FIG. 23 is another side schematic diagram of the oral camera shown in FIG. 20.

FIG. 24 is a side schematic diagram of a second dental arch and the oral camera shown in FIG. 20.

FIG. 25 is a schematic diagram of a second embodiment of the oral camera of the present disclosure.

FIG. 26 is a schematic diagram of the oral camera shown in FIG. 25, where the oral camera is placed on a first dental arch.

FIG. 27 is a schematic diagram of the oral camera shown in FIG. 25, where the oral camera is placed on a second dental arch.

FIG. 28 is a schematic diagram of the oral camera shown in FIG. 25, where the oral camera is placed on the second dental arch.

FIG. 29 is a schematic diagram of the oral camera shown in FIG. 25, where the oral camera is placed between the dental arches.

FIG. 30 is a schematic diagram of the oral camera shown in FIG. 25, where the oral camera faces the dental arches.

FIG. 31 is a schematic diagram of the oral camera in a rotation position.

FIG. 32 is a schematic diagram of the oral camera in another rotation position.

FIG. 33 is a schematic diagram of the oral camera and a light source.

FIG. 34 is a schematic diagram of a shooting range of the oral camera and a lighting range of the light source.

FIG. 35 is a schematic diagram of the oral imaging device according to a sixth embodiment of the present disclosure.

FIG. 36 is a schematic diagram of the oral imaging device according to the sixth embodiment of the present disclosure, where the mouth opener is removed.

### DETAILED DESCRIPTION

In order to make the purpose, technical solutions and features of the present disclosure clearly understood, exemplary embodiments of the present disclosure are described clearly and completely below in conjunction with the accompanying drawings in the exemplary embodiments of the present disclosure. Obviously, the exemplary embodiments described are only part of the embodiments of the present disclosure, not all of the embodiments. That is, the specific embodiments described herein are only used to explain the present disclosure, and are not used to limit the present disclosure.

The technical solutions in the embodiments of the present disclosure are clearly and completely described below in conjunction with the accompanying drawings in the embodiments of the present disclosure. Obviously, the described embodiments are only a part of the embodiments of the present disclosure, rather than all of the embodiments. Based on the embodiments of the present disclosure, all other embodiments obtained by those of ordinary skill in the art without creative work shall fall within the protection scope of the present disclosure.

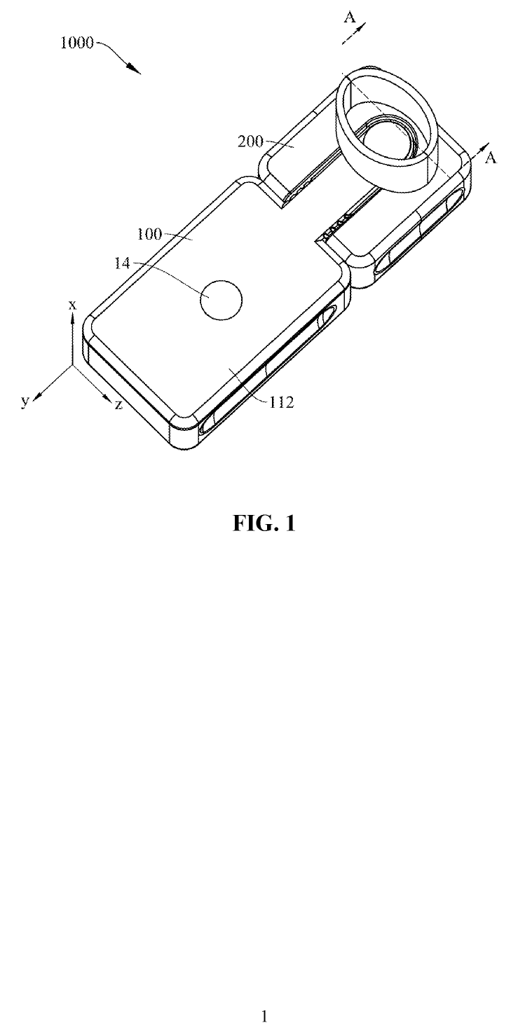

As shown in FIG. 1, FIG. 1 is a schematic diagram of an oral imaging device according to some embodiments of the present disclosure.

The oral imaging device 1000 of the embodiment of the present disclosure is configured to shoot an oral cavity of a user to obtain oral images, and the oral images include images of oral tissues such as teeth, gums, oral mucosa, and tongue.

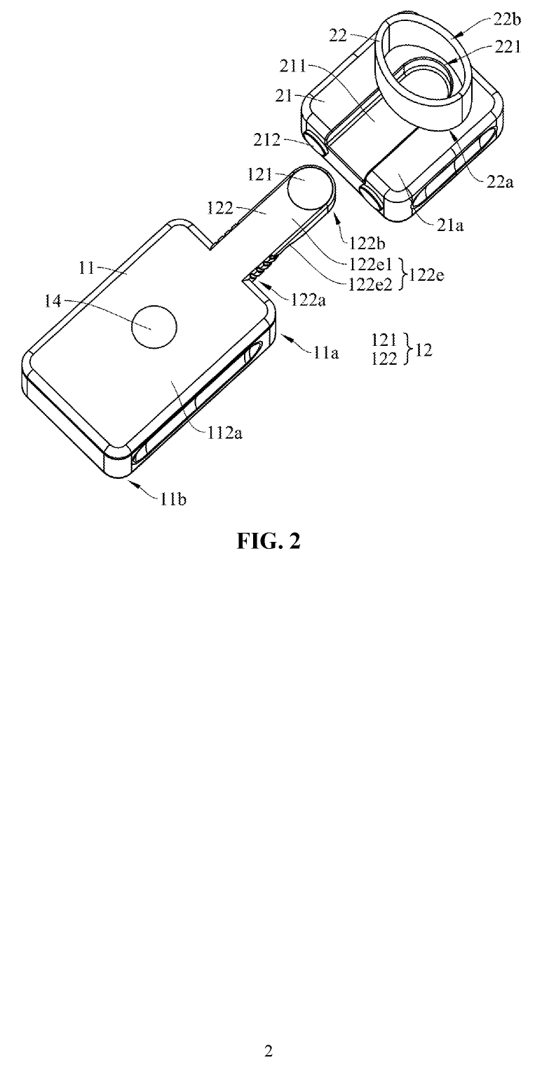

As shown in FIGS. 1 and 2, the oral imaging device 1000 in the embodiment of the present disclosure includes an oral camera 100 and a mouth opener 200. The oral camera 100 includes a main body 11 and a camera module 12 connected to the main body 11. The camera module 12 includes at least one camera 121. The mouth opener 200 has a first end 22a and a second end 22b opposite to the first end 22a. The mouth opener 200 includes an imaging through hole 221 penetrating through the first end 22a and the second end 22b. The second end 22b of the mouth opener 200 is configured to abut against an oral structure. For instance, the second end 22b of the mouth opener 200 is held in the mouth of the user.

In some embodiments, when shooting the outer surfaces of dental arches, the mouth opener 200 is configured to open the lips of a person being shot, and the imaging through hole 221 on the mouth opener 200 corresponds to the at least one camera 121 to ensure an imaging path. That is, the at least one camera 121 is able to capture an image of a part to be scanned via the imaging through hole 221.

A configuration of the mouth opener 200 prevents the lips from fitting with the gums. At this time, at least the outer surfaces of the teeth are corresponding to the imaging through hole 221, so that at least the outer surfaces of the teeth are within an imaging range of the at least one camera 121, so that the at least one camera 121 is able to obtain the image of at least the outer surfaces of the dental arches through the imaging through hole 221.

In some embodiments, the dental arches include two dental arches. As shown in FIG. 26, the two dental arches include a first dental arch 301 and a second dental arch 302. One of the first dental arch 301 and the second dental arch 302 is a lower dental arch, and the other one is an upper dental arch. Moreover, both of the first dental arch 301 and the second dental arch 302 include the teeth, or periodontal tissues such as the teeth and the gums.

In some embodiments, a position of the mouth opener 200 is variable relative to the at least one camera 121, so that the oral imaging device 1000 at least has the first form and the second form. In the first form, the at least one camera 121 faces the imaging through hole 221 and the camera module 12 does not exceed the second end 22b of the mouth opener 200. In the second form, the at least one camera 121 is exposed outside the imaging through hole 221 for being placed into the oral cavity. That is, in the first form, a position of the at least one camera 121 of the camera module 12 is corresponding to the position of the mouth opener 200, so that when the mouth opener 200 opens the lips, the outer surfaces of the teeth of the person being shot are fully exposed. At this time, the oral imaging device 1000 is at least configured to shoot the outer surfaces of the teeth, and a comprehensive image of the outer surfaces of the teeth having a good view angle is obtained. For instance, the at least one camera is allowed to take images horizontally. In the second form, the at least one camera 121 of the camera module 12 is inserted into the mouth of the person being shot, so as to shoot the occlusal surfaces and the inner surfaces of the teeth. Since the at least one camera 121 of the camera module 12 is able to be placed in the mouth, the at least one camera 121 of the camera module 12 is able to obtain images of the inside of the mouth, such as an image of the occlusal surfaces of molars.

In some embodiments, a display screen (not shown) is disposed on the main body 11 of the oral camera 100, so that an oral image captured by the at least one camera 121 is displayed in real time. In other embodiments, the oral camera 100 is communicated with a display device, so as to display the oral image captured by the at least one camera 121 on the display device.

Further, the oral camera 100 or the display device is able to analyze a current oral condition of the user based on the oral image captured. For instance, when the oral image captured is a tooth image, the teeth image may include but is not limited to an image of the occlusal surfaces of the tooth, an image of the inner surfaces of the teeth, and an image of the outer surfaces of the teeth, etc.

In some embodiments, the main body 11 is configured to power and control the camera module 12, transmit or store the images captured by the camera module 12, etc. For instance, the main body 11 includes a controller, a battery, a memory, and a communication element, etc. When the oral camera 100 works, the at least one camera 121 of the camera module 12 captures the oral images of at least one of the teeth and the gums in the oral cavity. The other components disposed inside the main body 11 are configured to analyze the current condition of the oral cavity of the user through the oral images captured by the at least one camera 121 of the camera module 12. Of course, the user is allowed to intuitively obtain the current oral condition through the oral images captured by the at least one camera 121 of the camera module 12. Moreover, the main body 11 is a portion of the oral camera 100 for the user to hold.

In some embodiments, the main body 11 further includes a power supply device (not shown). The power supply device is configured to be electrically connected to the camera module 12. In the first form and the second form, the power supply device is electrically connected to the camera module 12.

Specifically, the power supply device at least powers the camera module 12 to ensure that the camera module 12 operates normally in different working states (the first form and the second form), so that the camera module 12 works normally. For instance, the power supply device is a built-in rechargeable battery, such as a lithium-ion battery.

In some embodiments, the main body 11 is an arc-shaped handle that conforms to ergonomic design. For instance, the main body 11 is a columnar structure extending along a length direction, such as a cylindrical structure, so that the user or the dentist is able to hold the main body to adjust an angle and the position of the at least one camera 121 when taking the images.

In other embodiments, a shape of the main body 11 is not limited to a circle, and a square, rectangular, hexagonal, or another geometric shape that is easy to hold is adopted. For instance, the main body 11 is a block structure extending along the length direction, such as a cubic structure. Specifically, the cubic structure is a rectangular block structure or a square block structure.

The length direction of the main body 11 is an x-axis direction in FIG. 1, and the main body 11 further defines a width direction (e.g., a y-axis direction in FIG. 1) and a thickness direction (e.g., a z-axis direction in FIG. 1).

In some embodiments, long sides of the main body 11 in the length direction and wide sides in the width direction form a surface that is the largest surface of the main body 11. The at least one camera 121 and the largest surface of the main body 11 are on a same side or two opposite sides of the oral imaging device 1000.

Since the surface formed by the long sides and the wide sides of the main body 11 is the largest surface of the main body 11, the main body 11 as a whole presents a relatively flat shape. Further, the at least one camera 121 and the largest surface of the main body 11 are on the same side or the two opposite sides of the oral imaging device 1000, so that when the user holds the main body 11, the user generally holds on the largest surface and an opposite surface of the largest surface. A contact area between the largest surface and the palm of the user is larger, so the main body is comfortable to hold and is more conducive to the user maintaining a holding state of the oral imaging device 1000, ensuring the stability of the at least one camera 121. Furthermore, a lens of the at least one camera 121 is roughly parallel to the largest surface of the main body 11, which is easy to hold when in use.

In some embodiments, the at least one camera 121 is able to capture and receive light reflected from the internal parts of the oral cavity, and each of the images is formed by processing reflected light by the at least one camera 121. The reflected light received by the at least one camera 121 includes but is not limited to natural light, infrared light, and ultraviolet light. The at least one camera 121 may include only one camera or at least two cameras one (two or more), which is not limited in the present disclosure.

In one case, the at least one camera 121 is a two-dimensional camera 121. At this time, the at least one camera 121 collects light within the FOV (light of the part to be scanned) and transmits the light to an image sensor. The image sensor converts a light signal into an electrical signal, and after a series of conversions, a two-dimensional image is finally formed. It is understood that the image sensor is disposed in the camera module 12 or in the main body 11, which is not limited in the present disclosure. For instance, the at least one camera 121 is a two-dimensional infrared camera 121. At this time, the at least one camera 121 emits infrared light. The infrared light irradiates the part to be scanned and a part of the infrared light is reflected. The at least one camera 121 is able to capture reflected infrared light, and the image sensor converts the reflected infrared light into a digital signal. The digital signal is processed to generate an infrared image. For another example, the at least one camera 121 is a two-dimensional ultraviolet camera 121. In this case, the at least one camera 121 emits ultraviolet light. When the ultraviolet light is irradiated onto the part to be scanned, the internal parts of the oral cavity reflect a part of the ultraviolet light. The at least one camera 121 is able to capture reflected ultraviolet light. The reflected ultraviolet light is converted into the digital signal by the image sensor and the digital signal is processed to generate an ultraviolet image.

In another case, the at least one camera 121 is a time-of-flight camera. The time-of-flight camera first emits a laser pulse to illuminate the part to be scanned, and the laser pulse reaches the internal parts to be shot and is reflected back. The time-of-flight camera is able to calculate a distance from the part to be scanned to the time-of-flight camera by the time when the laser pulse is emitted and the time when the laser pulse is reflected back, so as to form a three-dimensional image. The present disclosure takes an example that the at least one camera 121 is the two-dimensional camera 121 for further illustration.

In some embodiments, the mouth opener 200 is not connected to at least one of the main body 11 and the camera module 12. The mouth opener 200 serves as an independent accessory. When shooting the outer surfaces of the dental arches, the second end 22b of the mouth opener 200 is configured to abut against the oral structure. For example, the second end 22b of the mouth opener 200 is held in the mouth of the user, and the first end 22a of the mouth opener 200 is configured to abut against the camera module 12 or the main body 11, and the imaging through hole 221 corresponds to the at least one camera 121 (at this time, the oral imaging device 1000 is in the first form), so that the at least one camera 121 is able to obtain an image of at least the outer surfaces of the dental arches through the imaging through hole 221 of the mouth opener 200.

In other embodiments, in the first form, the first end 22a of the mouth opener 200 is connected to the main body 11 or the camera module 12. The second end 22b of the mouth opener 200 is configured to abut against the oral structure. The at least one camera 121 is opposite to the imaging through hole 221 and does not exceed the second end 22b of the mouth opener 200 in an axial direction of the imaging through hole 221. That is, the at least one camera 121 is completely hidden in the imaging through hole 221, or the at least one camera 121 is flush with an end surface of the second end 22b of the mouth opener 200.

In this way, in the first form, when the mouth opener 200 is configured to open the lips, so the at least one camera 121 is able to shoot the outer surfaces of the teeth of the person being shot. The mouth opener 200 is connected and fixed to the main body 11 or the camera module 12, which prevents the mouth opener 200 from accidentally falling off, and facilitates a synchronous movement of the at least one camera 121 and the mouth opener 200 by moving the main body 11 during extraoral scanning, so as to avoid the misalignment of the at least one camera 121 and the imaging through hole 221 and affecting the shooting.

In some embodiments, in the first form, the mouth opener 200 is connected to at least one of the main body 11 and the camera module 12. The second end 22b of the mouth opener 200 abuts against the gums of the user to open the lips of the user. The at least one camera 121 is configured to shoot outside the oral cavity. When the user holds the second end 22b of the mouth opener 200 in the mouth, the second end 22b of the mouth opener 200 is located between the lips and the gums. The mouth opener 200 prevents the lips from fitting with the gums. At this time, at least the outer surfaces of the teeth correspond to the imaging through hole 221, and at least the outer surfaces of the teeth are located within the imaging range of the at least one camera 121. The at least one camera 121 is able to obtain a reflected signal of the part to be scanned (at least the outer surfaces of the dental arches) through the imaging through hole 221 to generate an image or a video.

In addition, by configuring the mouth opener 200, a certain distance is defined between the at least one camera 121 and the part to be scanned, so as to avoid a problem of blurring due to a too-close distance between the at least one camera 121 and the part to be scanned. When the oral camera 100 needs to scan the oral cavity, since the mouth opener 200 creates a certain distance between the part to be scanned and the at least one camera 121, the part to be scanned is located within the imaging range of the at least one camera 121, so that the image and/or the video taken by the at least one camera 121 is clear.

In the first form, the at least one camera 121 is located outside the oral cavity, and the at least one camera 121 is configured to shoot the outer surfaces of the teeth. Since the at least one camera 121 is outside the oral cavity, the user does not need to insert the oral imaging device into the oral cavity when using it, which reduces the interference with soft tissue in the oral cavity and potential discomfort. In addition, the at least one camera 121 in the first form is disposed outside the oral cavity, which makes the oral imaging device easy to operate, and is suitable for quickly checking the health of the outer side of the teeth, such as checking caries, plaque deposition or tooth arrangement.

In the second form, the at least one camera 121 is used inside the oral cavity. Specifically, in the second form, the user holds the main body 11 and inserts the at least one camera 121 into the oral cavity. The at least one camera 121 is configured to shoot inside the oral cavity. For example, the at least one camera 121 is aligned with the inner surfaces and the occlusal surfaces of the teeth, so as to obtain images of the inner surfaces and the occlusal surfaces of the teeth.

The oral imaging device 1000 in the embodiment of the present disclosure realizes an extraoral scanning function and an intraoral scanning function. That is, the oral imaging device 1000 is an intraoral and extraoral two-in-one oral imaging device 1000. An intraoral oral imaging device is a device where the at least one camera 121 is inserted into the oral cavity (i.e, the at least one camera 121 exceeds inner surfaces of front teeth). An extraoral oral imaging device is a device where the at least one camera 121 is used outside the oral cavity (i. e, the at least one camera 121 does not exceed the inner surfaces of front teeth).

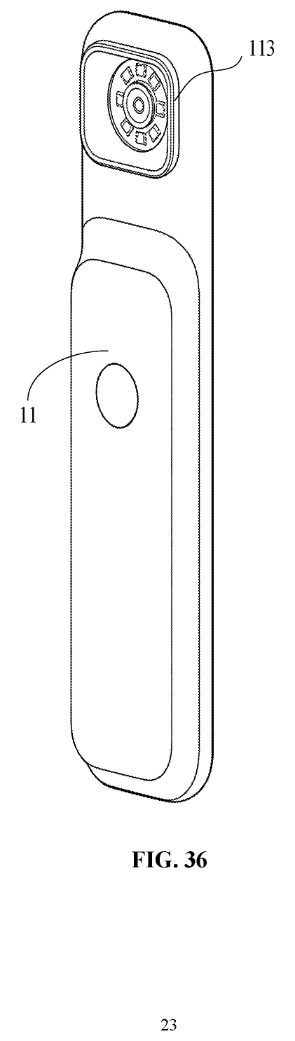

In some embodiments, as shown in FIGS. 35-36, the mouth opener 200 is detachably connected to the camera module 12 and is disposed around the at least one camera 121. Specifically, the main body comprises a connecting component 113 disposed around the at least one camera. In the first form, the first end 22a of the mouth opener 200 is detachbaly connected with the connecting component 113, so that the mouth opener 200 is disposed around the at least one camera 121 in the first form. The connection method between the connecting component 113 and the mouth opener is nit limited thereto. For example, the connecting component 113 is magnetically connected.to the mouth opener. in this case, the mouth opener 200 is easily positioned and is also easily removed. For another example, an engaging groove is disposed on the first end 22a of the mouth opener 200, and an engaging rim is defined on the at least one camera 121. The engaging rim is detachably engaged with the engaging groove to detachably connect the mouth opener 200 to the connecting component 121. In the firt form, the mouth opener is disposed around the at least one camera, and the at least one camera is exposed from the imaging through hole 221. At this point, the at least one camera 121 is at least configured to shoot the outer surfaces of the dental arches. In the second form, the mouth opener 200 is directly removed from the at least one camera 121, thereby enabling the at least one camera 121 to shoot at least one of the occlusal surfaces and/or the inner surfaces of the dental arches. Of course, in other embodiments, the engaging rim is defined on the first end of the mouth opener 200, while the engaging groove is disposed around at least one camera..

In some embodiments, in the first form, the at least one camera 121 is at least configured to shoot the outer surfaces of the dental arches. In the second form, the at least one camera 121 is at least configured to shoot at least one of the occlusal surfaces and/or the inner surfaces of the dental arches. Specifically, in the first form, the mouth opener 200 abuts against the gums and supports the lips, the outer surfaces of the teeth face the imaging through hole 221, and the at least one camera 12 at least receives energy reflected back from the outer surfaces of the dental arches and shoots an image. In the second form, the at least one camera 121 is inserted into the oral cavity to shoot images of the inner surfaces and the occlusal surfaces of the teeth, as well as a partial image of a single tooth, thereby achieving comprehensive shooting of the occlusal surfaces, the inner surfaces and the outer surfaces of the teeth.

In some embodiments, at least one of the position of the at least one camera 121 and the position of the mouth opener 200 is variable relative to the main body 11, so that the oral imaging device 1000 is able to switch between the first form and the second form. Therefore, in the first form, the lips are opened by the mouth opener 200 to expose the outer surfaces of the teeth of the person being shot. At this time, the oral imaging device 1000 is at least configured to shoot the outer surfaces of the teeth, and a comprehensive image of the outer surfaces of the teeth having a good view angle is obtained. For instance, the comprehensive image of the outer surfaces of the teeth is obtained by aligning the at least one camera and the teeth horizontally. In the second form, the at least one camera 121 is exposed from the imaging through hole 221 of the mouth opener 200, and the at least one camera 121 of the camera module 12 is inserted into the mouth of the person being shot. The at least one camera 121 is movable in the mouth, and a distance and an angle between the at least one camera 121 and the teeth are freely changed, so as to take different oral images. For example, the at least one camera 121 is able to shoot the occlusal surfaces and the inner surfaces of the teeth. Since the at least one camera 121 of the camera module 12 is inserted into the mouth, the at least one camera 121 of the camera module 12 is able to take images of the internal parts of the mouth, such as the images of the occlusal surfaces of the molars.

Specifically, the oral imaging device 1000 is able to switch between the first form and the second form by changing a positional relationship between the mouth opener 200 and the main body 11, or by changing a positional relationship between the camera module 12 and the main body 11, or by changing a positional relationship of the mouth opener 200 and the camera module 12 and the main body 11. For instance, the mouth opener 200 may move relative to the main body 11 or may be separated from the main body to change the positional relationship thereof, and the camera module 12 may be telescopic, rotated or disassembled relative to the main body 11 to change the positional relationship thereof.

In the first form, the mouth opener 200 supports the lips to facilitate the shooting of the outer surfaces of the teeth; while in the second form, the at least one camera 121 is inserted into the oral cavity to shoot the inner surfaces and the occlusal surfaces of the teeth. The oral imaging device 1000 is able to switch between the first form and the second form, thereby shooting at least the images of the outer surfaces, the inner surfaces, and the occlusal surfaces of the teeth. Namely, different oral images are obtained by only one oral imaging device 1000, so that the oral imaging device 1000 meets needs of comprehensively shooting various internal parts in the oral cavity.

In some embodiments, in the first form, the at least one camera 121 is aligned with the imaging through hole 221 of the mouth opener 200, and the at least one camera 121 is located outside the oral cavity. The at least one camera 121 is directly aligned with the outer surfaces of the teeth through the imaging through hole 221, thereby receiving the light signal reflected back from the outer surfaces of the teeth through the imaging through hole 221. At this time, the imaging through hole 221 acts as an optical channel to ensure that the at least one camera 121 clearly captures the image of the outer surfaces of the teeth, while the mouth opener 200 supports the lips to provide a stable shooting environment for the at least one camera 121.

In some embodiments, in the second form, the mouth opener 200 is connected to the main body 11, and the at least one camera 121 is staggered with the imaging through hole 221 to be exposed outside the imaging through hole 221, so that the imaging through hole 221 is no longer within the imaging range of the at least one camera 121, avoiding the interference of the mouth opener 200 on the at least one camera 121. At this time, the at least one camera 121 is inserted into the oral cavity to align with the inner surfaces and the occlusal surfaces of the teeth for shooting.

In some embodiments, in the second form, the at least one camera 121 exceeds the imaging through hole 221 and is exposed outside the imaging through hole 221. At this time, the mouth opener 200 is disposed between the at least one camera 121 and the main body 11, and the at least one camera 121 is no longer disposed inside the mouth opener 200, thereby facilitating the at least one camera 121 to insert into the oral cavity to obtain the images of the internal parts of the oral cavity, such as shooting the occlusal surfaces and the inner surfaces of the dental arches.

In some embodiments, in the second form, the mouth opener 200 is separated from the main body 11, and the at least one camera 121 protrudes relative to the main body 11 to be inserted into the oral cavity. Specifically, the mouth opener 200 is separated from the main body 11, that is, the mouth opener 200 is removed from the main body 11, and the at least one camera 121 protrudes relative to the main body 11, so that the at least one camera 121 is freely inserted into the oral cavity to obtain the oral images of the oral cavity. That is, the at least one camera 121 is able to take images of the occlusal surfaces and the inner surfaces of the dental arches.

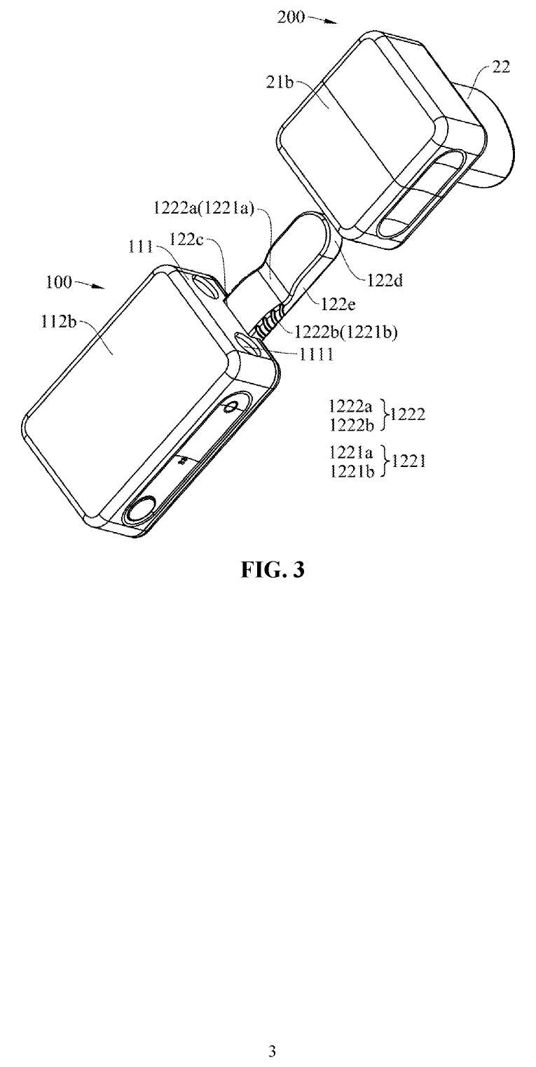

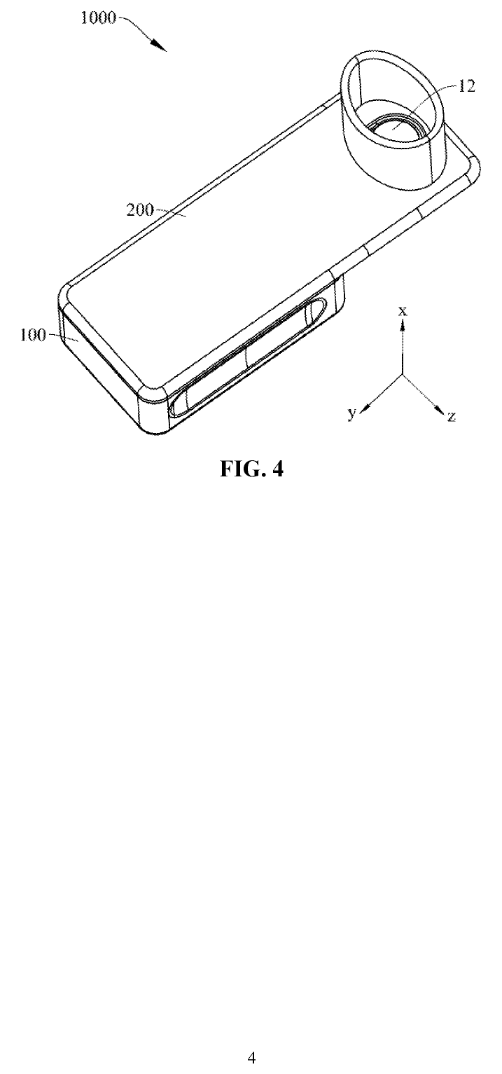

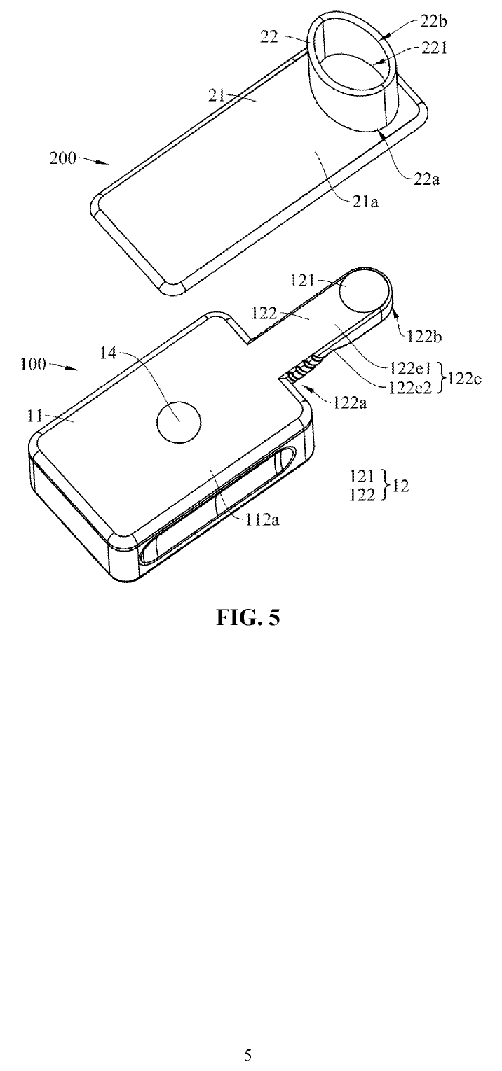

In some embodiments, as shown in FIGS. 1-5, the mouth opener 200 is detachably connected to the main body 11 or the camera module 12. That is, the mouth opener 200 is able to be connected to the main body 11 or the camera module 12 and is able to be detached from the main body 11 or the camera module 12, so that the oral imaging device 1000 is able to switch between the first form and the second form. At this time, the first form is a form where the mouth opener 200 is connected to the main body 11 or the camera module 12, and the at least one camera 121 is able to receive the light signal of the inner surfaces of the teeth passing through the imaging through hole 221. The second form is a form where the mouth opener 200 is detached from the main body 11 or the camera module 12.

The mouth opener 200 is detachably connected to the main body 11 or the camera module 12, so that the oral imaging device 1000 is able to flexibly switch between two different forms to meet different shooting requirements. In the first form, the mouth opener 200 is connected to the main body 11, and the at least one camera 121 is aligned with the imaging through hole 221 of the mouth opener. Since the imaging through hole 221 penetrates through the first end and the second end of the mouth opener 200, when the mouth opener 200 is placed in the oral cavity, the outer surfaces of the teeth face the imaging through hole 221. The at least one camera 121 receives the light signal reflected from the outer surfaces of the teeth through the imaging through hole 221, thereby capturing the image of the outer surfaces of the teeth and realizing image acquisition.

In the second form, the mouth opener 200 is separated from the main body 11, and the at least one camera 121 protrudes relative to the main body 11. At this time, the at least one camera 121 is no longer facing the imaging through hole 221 of the mouth opener 200. Since a support piece 122 has a certain length, the at least one camera 121 is freely inserted into the oral cavity, thereby obtaining the light signal reflected back from the inner surfaces and the occlusal surfaces of the teeth and forming the image of the inner surfaces and the occlusal surfaces of the teeth.

Optionally, the mouth opener 200 is detachably connected to the main body 11 or the camera module 12 by at least one of magnetic attraction connection, snap-fit connection, and sleeving. The oral imaging device 1000 is detachably connected to the mouth opener 200 and the main body 11 when in use, so that the oral imaging device 1000 is able to switch between the first form and the second form.

The magnetic attraction connection uses magnetic force to realize connection and separation of the mouth opener 200 and the main body 11. The mouth opener 200 and the main body 11 are automatically aligned and connected without external force, saving time and manpower, and reducing wear and looseness problems that may occur by mechanical connection.

In another embodiment, the mouth opener 200 and the main body 11 are quickly assembled and disassembled by the snap-fit connection. The snap-fit connection does not require additional locking accessories, thereby saving costs. In addition, the snap-fit connection further enables the high connection strength and reliability between the mouth opener 200 and the main body 11, so that the mouth opener 200 and the main body 11 are allowed to be disassembled and assembled repeatedly.

In another embodiment, the mouth opener 200 and the main body 11 are quickly assembled and disassembled by sleeving. The mouth opener 200 and the main body 11 are sleeved with each other without additional locking accessories, thereby saving costs.

In some embodiments, the main body 11 includes a first end 11a and a second end 11b opposite to the first end 11a. The first end 11a and the second end 11b of the main body 11 are disposed in the length direction thereof. The at least one camera 121 is disposed on an end surface of the first end 11a of the main body 11, and an optical axis of the at least one camera 121 is disposed at an angle to the length direction of the main body 11. The mouth opener 200 is detachably sleeved on an outer periphery of the first end 11a of the main body 11, so that the oral imaging device 1000 is able to switch between the first form and the second form.

Thus, in the first form, when the at least one camera 121 is disposed on the end surface of the main body 11 in the length direction (i.e., the end surface of the first end 11a of the main body 11), when the user uses the oral imaging device 1000, the user needs to hold the main body 11 vertically, so that the length direction of the main body 11 is roughly perpendicular to a horizontal plane, and the width direction and thickness direction of the main body 11 are roughly parallel to the horizontal plane. At this time, the end surface where the at least one camera 121 is disposed is naturally aligned with the oral cavity, and the user is able to take the oral images without raising his/her arm or adjusting the angle of the main body 11. The main body 11 is not only natural and comfortable for the user to hold, but also reduces arm fatigue brought to the user, which helps the at least one camera 121 to stably align with the internal parts in the oral cavity and obtain clear and stable oral images.

In some embodiments, the first end 11a of the main body 11 is bent so that the optical axis of the camera module 12 is disposed at an angle to the length direction of the main body 11. A bending portion is formed between the first end 22a and the second end 22b of the mouth opener 200, and the bending portion is sleeved on a bending portion of the first end of the main body 11. The first end 22a of the mouth opener 200 extends to the outer periphery of the main body 11, and an axis of the imaging through hole 221 at the second end 22b of the mouth opener extends along the optical axis of the camera module 12. In this way, the bending portion on the main body 11 and the bending portion on the mouth opener 200 are matched with each other to avoid the mouth opener 200 from easily detaching from the first end 11a of the main body 11, thereby improving the stability of the mouth opener 200 on the main body 11, and reducing a risk of accidental drop and damage of the mouth opener 200.

When the mouth opener 200 is detachably connected to the main body 11 or the camera module 12, and the mouth opener 200 is made of a blister material. Specifically, the mouth opener 200 is made of the blister material, which means that the mouth opener 200 itself is a disposable item that needs to be replaced. The blister material includes, but is not limited to, polyvinyl chloride, polyethylene terephthalate, polypropylene or polystyrene, etc., which is not limited in the present disclosure. The mouth opener 200 made of the blister material is served as the disposable item, and the mouth opener is discarded after use by the user to avoid a risk of cross infection. The cost of the blister material is low, which reduces costs.

In some embodiments, the camera module 12 further includes the support piece 122 connected to the main body 11, and the at least one camera 121 is disposed on the support piece 122. The support piece 122 is disposed along the length direction of the main body 11, that is, a length direction of the support piece 122 is the length direction of the main body 11, a thickness of the support piece 122 is the thickness direction of the main body 11, and a width direction of the support piece 122 is the width direction of the main body 11.

Specifically, the support piece 122 inc includes a connecting end 122a and an insertion end 122b opposite to the connecting end in the length direction. The connecting end 122a of the support piece 122 is connected to the main body 11, the insertion end 122b is at least partially inserted into the oral cavity, and the at least one camera 121 is disposed at the insertion end 122b of the support piece 122.

Since the support piece 122 has a certain length, in the second form, it is convenient for the at least one camera 121 to freely insert into the oral cavity, so as to obtain the light signal reflected back from the inner surfaces and the occlusal surfaces of the teeth and form the image of the inner surfaces and the occlusal surfaces of the teeth.

The support piece 122 is configured to connect the main body 11 and the at least one camera 121. The support piece 122 further provides a mounting space for the at least one camera 121. In one example, the support piece 122 is detachably connected to the main body 11, such as the support piece 122 is snapped with or screwed with the main body 11. In another example, the support piece 122 is fixedly connected to the main body 11, such as the support piece and the main body are fixed by one-piece molding, bonding or welding. The at least one camera 121 may be detachably or fixedly connected to the support piece 122, which is not limited thereto. The support piece 122 connects the main body 11 and the at least one camera 121, which realizes an indirect connection between the at least one camera 121 and the main body 11. Compared with a case where the at least one camera 121 is directly connected to the main body 11, the position of the at least one camera 121 is variable relative to the main body 11. That is, the position of the at least one camera 121 connected to the main body 11 is not related to the position of the mouth opener 200, and the at least one camera 121 is allowed to protrude relative to the main body 11. Namely, the at least one camera 121 is able to stretch outside the main body 11, and the position of the at least one camera 121 relative to the main body 11 is variable, which is convenient for the at least one camera 121 to be inserted into the oral cavity.

In some embodiments, the support piece 122 is detachably mounted on the main body 11, which is convenient for replacement, cleaning or storage. In other embodiments, the support piece 122 is configured as a part of the main body 11.

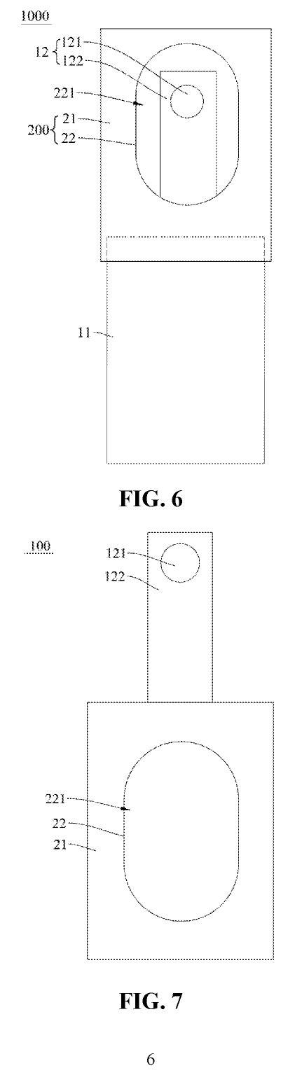

In some embodiments, as shown in FIGS. 6 and 7, the mouth opener 200 is slidably connected to the main body 11 or the camera module 12.

Specifically, when the mouth opener 200 slides to a first position shown in FIG. 6, the oral imaging device 1000 is in the first form, and the at least one camera 121 is aligned with the imaging through hole 221 of the mouth opener 200. When the mouth opener 200 slides to a second position shown in FIG. 7, the oral imaging device 1000 is in the second form, the at least one camera 121 protrudes relative to the main body 11, and the at least one camera 121 is staggered with the imaging through hole 221 of the mouth opener 200.

Specifically, the mouth opener 200 is able to slide along a certain direction (e.g., the length direction of the main body 11) of the main body 11 to switch the oral imaging device 1000 between the first form and the second form. There are mul22le sliding connection methods for the mouth opener 200, which are not limited in the present disclosure.

For example, a sliding groove or a guide rail is disposed on the main body 11 or the support piece 122, and the mouth opener 200 includes a sliding block corresponding to the sliding groove or a guide piece corresponding to the guide rail, so that the mouth opener 200 is slidable on the main body 11 or the camera module 12. Alternatively, the mouth opener 200 is sleeved on at least one of the outer periphery of the main body 11 and an outer periphery of the camera module 12, so that the mouth opener 200 is slidably connected to at least one of the main body 11 and the camera module 12.

For example, when the mouth opener 200 is in the first position, that is, the oral imaging device 1000 is in the first form, the at least one camera 121 is aligned with the imaging through hole 221 of the mouth opener 200, and the at least one camera 121 is able to capture the image of the outer surfaces of the teeth through the imaging through hole 221 of the mouth opener 200. When it is necessary to switch the oral imaging device 10000 to the second form, the user only needs to push or pull the mouth opener 200 along a sliding direction to slide the mouth opener 200 to the second position, the imaging through hole 221 of the mouth mouth opener 200 is staggered with the at least one camera 121, and the support piece 122 and the at least one camera 121 protrude relative to the main body 11, thereby allowing the at least one camera 121 to insert into the oral cavity to shoot the inner surfaces and the occlusal surfaces of the teeth. The mouth opener is slidably connected, which is relatively simple in structure, has a low cost, and is fast and flexible. The user is able to easily switch the oral imaging device between the first form and the second form according to different shooting needs.

In some embodiments, the support piece 122 stretches and protrudes relative to the main body 11 in a direction away from the main body 11, and the mouth opener 200 slides relative to the main body 11 in an extension direction of the support piece 122.

When the oral imaging device 1000 shoots the outer surfaces of the teeth in the first form, the support piece 122 and the main body 11 are disposed roughly in a longitudinal direction (i.e., a vertical direction), and the extension direction of the support piece 122 relative to the main body 11 is roughly in the longitudinal direction, while the oral imaging device 1000 is movable in a transverse direction (i.e., the horizontal direction) to shoot different positions of the outer surfaces of the teeth, so that the mouth opener 200 slides relative to the main body 11 in the extension direction of the support piece 122. A sliding direction of the mouth opener 200 is different from a moving direction of the oral imaging device 1000 to shoot different positions of the outer surfaces of the teeth. The sliding direction of the mouth opener 200 is roughly perpendicular to the moving direction of the oral imaging device 1000, thereby effectively preventing the mouth opener 200 from sliding relative to the main body 11 or the camera module 12 when the oral imaging device 1000 moves in the transverse direction to shoot different positions of the outer surfaces of the teeth. Thus, the imaging through hole 221 is not staggered with the at least one camera 121 when the main body moves in the horizontal direction.

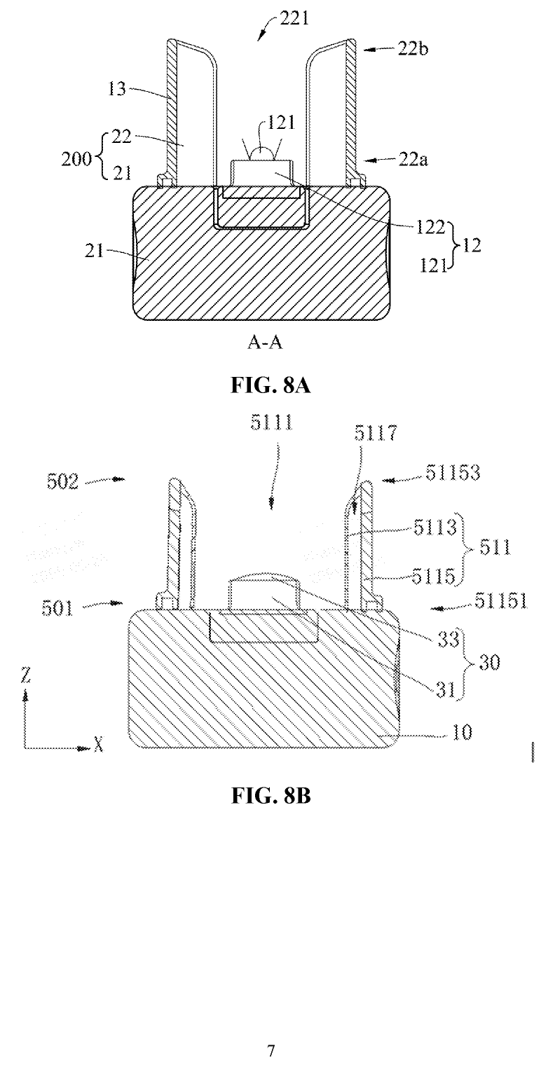

In some embodiments, as shown in FIGS. 8A-11, the camera module 12 is retractably connected to the main body 11 so that the oral imaging device 1000 is able to switch between the first form and the second form.

Optionally, the camera module 12 realizes a retractable connection relative to the main body 11 by moving, sliding, lifting, etc. A connection structure thereof of moving, sliding, lifting, etc. is relatively simple, low-cost, fast and flexible. The user is able to switch the oral imaging device 1000 between the first form and the second form according to different shooting requirements.

In some embodiments, the camera module 12 is slidably connected to the main body 11. When the camera module 12 slides to a position where the at least one camera 121 is corresponding to the imaging through hole 221, the oral imaging device 1000 is in the first form, and the at least one camera 121 is aligned with the imaging through hole 221 of the mouth opener 200. When the camera module 12 slides to a position where the at least one camera 121 and the imaging through hole 221 are staggered, the oral imaging device 1000 is in the second form, the support piece 122 and the at least one camera 121 protrude relative to the main body 11, and the at least one camera 121 is able to be inserted into the oral cavity.

Specifically, the camera module 12 is slidable along a certain direction (e.g., the length direction of the main body) of the main body 11, so the camera module 12 is slidably connected to the main body 11. The camera module 12 is slidably connected to the main body 11 through different ways, which are not limited in the present disclosure. For example, the sliding groove or the guide rail is disposed on the main body 11 or the support piece 122, and the mouth opener 200 includes the sliding block corresponding to the sliding groove or the guide piece corresponding to the guide rail.

When the camera module 12 slides to enable that the at least one camera 121 is aligned with the imaging through hole 221 of the mouth opener 200, the oral imaging device 1000 is in the first form. At this time, the at least one camera 121 is able to capture the image of the outer surfaces of the teeth through the imaging through hole 221 of the mouth opener 200. When it is necessary to switch the oral imaging device 10000 to the second form, the user only needs to push or pull the camera module 12 along a sliding direction to slide the camera module 12 to switch the first form to the second form. At this time, the support piece 122 and the at least one camera 121 protrude relative to the main body 11, and the imaging through hole 221 of the mouth mouth opener 200 is staggered with the at least one camera 121, thereby allowing the at least one camera 121 to insert into the oral cavity to shoot the inner surfaces and the occlusal surfaces of the teeth. The camera module 12 is slidably connected, which is relatively simple in structure, has a low cost, and is fast and flexible. The user is able to easily switch the oral imaging device between the first form and the second form according to different shooting needs.

In some embodiments, the camera module 12 is embedded in the main body 11 and is slidable in the main body 11. Specifically, the support piece 122 of the camera module 12 is sleeved on the main body 11, and the camera module 12 is slidable on the main body 11 along a specific direction (such as the length direction), so as to realize the rapid switching of the oral imaging device 1000 between different forms.

Specifically, when the oral imaging device 1000 is in the first form, the camera module 12 is stored in the main body 11 or is close to the main body 11 through the sliding connection, so that an overall volume of the oral imaging device 1000 is small, which is convenient for handheld operation and storage. At this time, the at least one camera 121 is aligned with the imaging through hole 221 of the mouth opener 200 to capture the image of the outer surfaces of the teeth.

When it is necessary to switch the oral imaging device 1000 to the second form, the camera module 12 extends outward by sliding, and the at least one camera 121 protrudes from the main body 11, so that the at least one camera 121 is allowed to be inserted into the oral cavity to capture the inner surfaces and the occlusal surfaces of the teeth.

In some embodiments, the camera module 12 is stretchable in a direction perpendicular to the axial direction of the imaging through hole 221. For example, the camera module 12 is stretchable in the length direction or the width direction of the support piece 122 to perform linear motion.

Specifically, when the support piece 122 is retracted to the first position, the oral imaging device 1000 is in the first form, and the at least one camera 121 is aligned with the imaging through hole 221 of the mouth opener 200. When the support piece 122 is stretched to the second position, the oral imaging device 1000 is in the second form, the mouth opener 200 is connected to the main body 11, and the at least one camera 121 is staggered with the imaging through hole 221 of the mouth opener 200, so that the at least one camera 121 is allowed to be inserted into the oral cavity.

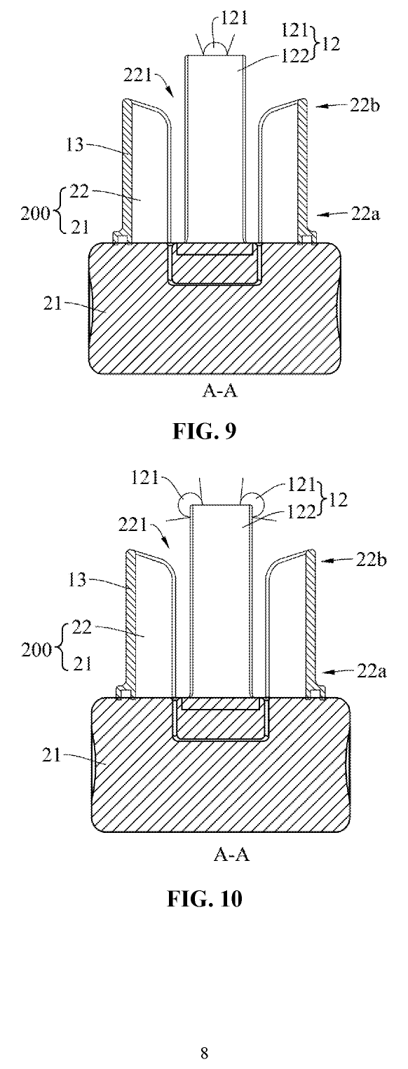

In other embodiments, as shown in FIGS. 8A-11, the camera module 12 is stretchable along the axial direction of the imaging through hole 221. Specifically, when the support piece 122 is retracted to the first position, the oral imaging device 1000 is in the first form. As shown in FIG. 8, the at least one camera 121 is aligned with the imaging through hole 221 of the mouth opener 200, and the at least one camera is located in the imaging through hole. When the support piece 122 is stretched to the second position, the oral imaging device is in the second form. As shown in FIGS. 9 and 10, the mouth opener 200 is connected to the main body 11, and the support piece 122 protrudes outside the imaging through hole 221 in the axial direction of the imaging through hole 221, so that the mouth opener 200 is located between the main body 11 and the at least one camera 121, and the at least one camera 121 is allowed to be inserted into the oral cavity.

When the oral imaging device 1000 is in the first form, the support piece 122 and the at least one camera 121 are retracted inside the main body 11, the support piece 122 and the at least one camera 121 do not stretch from the second end 22b of the mouth opener 200, and the at least one camera 121 is aligned with the imaging through hole 221 of the mouth opener 200. At this time, most of the support piece 122 is stored inside the main body 11, and the at least one camera 121 faces the outer surfaces of the teeth through the imaging through hole 221 to capture the image of the outer surfaces of the teeth. Such design makes the oral imaging device 1000 have a compact structure in the first form, which is convenient for the user to hold and operate, while ensuring the stability and clarity of the shooting.

When the oral imaging device 1000 needs to switch to the second form, the support piece 122 moves from the first end 22a to the second end 22b of the mouth opener 200 and stretches outward, so that the at least one camera 121 finally stretches from the imaging through hole 221 and exceeds the second end 22b of the mouth opener 200. At this time, the mouth opener 200 is still connected to the main body 11, but the support piece 122 is fully stretched, and the at least one camera 121 is allowed to be inserted into the oral cavity to shoot the inner surfaces and the occlusal surfaces of the teeth. The retractable mechanism not only realizes the rapid switching between the first form and the second form of the oral imaging device 1000, but also ensures the stability and reliability of the support piece 122 during a retracting process. As a result, the oral imaging device 1000 is allowed to flexibly switch between the two forms to meet different shooting needs.

In some embodiments, as shown in FIGS. 8A-11, the oral imaging device 1000 further includes an isolating sleeve 13 detachably sleeved on an inner side and an outer side of the mouth opener 200 from the second end 22b of the mouth opener 200, and the isolating sleeve 13 is configured to isolate the mouth opener 200 and the oral cavity.

That is, the isolating sleeve 13 is configured to isolate the oral cavity from the mouth opener 200 to prevent the oral cavity from directly contacting the mouth opener 200. The isolating sleeve 13 covers an outer side and an inner side of the mouth opener 200. In the first form, when the user uses the oral imaging device 1000, the isolating sleeve 13 prevents the oral cavity from contacting the outer side and the inner side of the mouth opener 200, thereby effectively avoiding hygienic problems that may be caused by different users cross-using the oral imaging device 1000.

Moreover, the isolating sleeve 13 provides good isolation between different users, prevents cross-infection or bacterial transmission caused by a situation where different users direct the user the mouth opener 200 to directly abut against the dental arches, and improves the hygienic safety of the oral imaging device 1000.

In some embodiments, the isolating sleeve 13 covers at least part of the main body 11. That is, the isolating sleeve 13 at least partially covers the main body 11. In the first form, when the user uses the oral imaging device 1000, the isolating sleeve 13 prevents the oral cavity of the user from contacting at least part of the main body 11, thereby avoiding cross-infection problems.

In addition, the isolating sleeve 13 covers at least part of the main body 11, prevents the oral cavity of the user from contaminating the main body 11, prevents cross-infection or bacterial transmission caused by direct contact between different users and the main body 11, and improves the hygiene and safety of the oral imaging device 1000.

In some embodiments, the isolating sleeve 13 is made of the blister material.

It is understood that the isolating sleeve 13 must be safe enough for being inserted into the mouth. Since the isolating sleeve 13 covers the outer side and the inner side of the mouth opener 200, the mouth opener 200 supports the isolating sleeve 13. Therefore, the isolating sleeve 13 has lower requirements for hardness and thickness. For instance, the isolating sleeve 13 is made of the blister material. The blister material includes but is not limited to the polyvinyl chloride, the polypropylene or the polystyrene.

Then, the isolating sleeve 13 made of the blister material is served as a disposable item, and the isolating sleeve 13 is discarded after use, thereby avoiding the risk of cross infection. The blister material is low in cost, which reduces the cost of the isolating sleeve 13.

In some embodiments, the isolating sleeve 13 is an integrated component. In this way, joint seams are omitted, and liquid (such as saliva) is prevented from contacting the mouth opener 200 through the isolating sleeve, thereby effectively preventing the oral cavity of the user from contacting the outer side and the inner side of the mouth opener 200, thereby effectively avoiding the hygienic problems that may be caused by different users cross-using the oral imaging device 1000.

In some embodiments, the imaging through hole 221 defines a cross section of in the axial direction thereof. A length of the cross section of the imaging through hole 221 in a first direction is greater than a length of the cross section of the imaging through hole in a second direction. The first direction is perpendicular to the second direction. The first direction extends in the width direction of the main body 11, and the second direction extends along the length direction of the main body 11.

It is understood that the cross section of the imaging through hole 221 perpendicular to the axial direction of the imaging through hole 221 includes a long axis with a larger dimension in the first direction and a short axis with a dimension less than the long axis, and the short axis is in the second direction. A dimension of the main body 11 in the length direction is larger than a dimension of the main body 11 in the width direction. A dimension of the imaging through hole 221 in the length direction of the main body 11 is less than a dimension of the imaging through hole 221 in the width direction of the main body 11. It is noted that the long axis of the imaging through hole 221 overlaps the short axis of the main body 11 in the width direction of the main body 11, and the short axis of the imaging through hole 221 overlaps the long axis of the main body 11 in the length direction of the main body 11. When the user holds the mouth opener 200 in the mouth, the long axis of the imaging through hole 221 needs to be placed into the oral cavity parallel to the horizontal plane to adapt to the shape of the oral cavity in an open state. Therefore, in the first form, when the user uses the oral imaging device 1000, the user holds the oral imaging device 1000 vertically. In a vertical holding state, the long axis of the main body 11 is perpendicular to the horizontal plane. When the at least one camera 121 is disposed on one side of the support piece 122, the user needs to hold the main body 11 vertically so that the length direction of the main body 11 is roughly perpendicular to the horizontal plane, and the width direction and the thickness direction are roughly parallel to the horizontal plane. At this time, the side of the support piece 122 where the at least one camera 121 is disposed is naturally aligned with the oral cavity of the user, and the user is allowed to take images without raising his/her arm or adjusting the angle of the main body 11. The user is allowed to hold the oral imaging device 1000 naturally and comfortably and further reduce arm fatigue, which helps the at least one camera 121 to aim at internal parts of the oral cavity more stably and obtain clear and stable oral images.

In some embodiments, as shown in FIG. 10, the camera module 12 includes two cameras 121 and the support piece 122. That is, the at least one camera 121 includes the two cameras 121. The support piece 122 is retractable. The support piece 122 is connected to the main body 11, and the support piece 122 is retractable relative to the main body 11 along the axial direction of the imaging through hole 221. The two cameras 121 are disposed on the support piece 122, and the two cameras 121 are disposed along the first direction. Optical axes of the two cameras 121 are disposed at an angle to the axial direction of the imaging through hole 221. The optical axes of the two cameras 121 are not parallel to the first direction.

In this way, by using the two cameras 121 whose optical axes are inclined relative to the axial direction of the imaging through hole 221, the shooting range of the camera module 12 is increased. In the first form, the two cameras are exposed from the imaging through hole, so the outer surfaces of the teeth are shot. In the second form, enough tooth surfaces are shot at one time, so that more inner surfaces and occlusal surfaces in the oral cavity are shot at one time; thereby reducing the number of shoots and the difficulty of shooting. Further, low efficiency of image acquisition caused by adjusting the shooting angle multiple times is avoided, thereby improving the shooting efficiency. In addition, in the second form, the two cameras 121 are respectively disposed on two sides of the support piece 122 in the thickness direction thereof, and the two cameras 121 simultaneously shoot an image of the teeth located in an upper jaw and an image of the teeth located in a lower jaw, which ensures that the viewing angle and shooting angle of the two images are similar. Therefore, the image of the teeth located in the upper jaw and the image of the teeth located in the lower jaw have the same reference datum in space, which is convenient for occlusal analysis, evaluation of tooth alignment, and identification of potential problems.

In some embodiments, an angle between the optical axes of the two cameras 121 is not less than 60°. Specifically, the optical axes of the two cameras 121 define the angle therebetween. An opening of the angle between the optical axes of the two cameras 121 is away from the main body 11, and the angle between the optical axes of the two cameras 121 is not less than 60°.

The optical axes of the two cameras 121 define the angle, which means that the imaging directions (the optical axes) of the two cameras 121 are not parallel to each other, but form a certain angle. For example, when the two cameras 121 are respectively disposed on two sides of the support piece 122 in the thickness direction thereof, the optical axes of the two cameras 121 are completely opposite to each other and face opposite directions, and the angle between the optical axes of the two cameras 121 is 180°. The opening of the angle between the optical axes of the two cameras 121 is away from the main body 11, which means that the angle formed by the optical axes of the two cameras 121 is away from the main body 11. That is, the imaging directions (optical axis) of the two cameras 121 are spread outward, so that the light received by the two cameras 121 i from a direction away from the main body 11. When the angle between the optical axes of the two cameras 121 is less than 60°, imaging ranges of the two cameras 121 are limited on the one hand, and imaging ranges of the two cameras 121 are at least partially coincided. The angle between the optical axes of the two cameras 121 is not less than 60°, which makes full use of the imaging ranges of the two cameras 121 and improves the imaging efficiency of the camera module 12.

In addition, when the angle between the optical axes of the two cameras 121 is not less than 60, the two cameras 121 have a wider coverage. For example, a first camera 121 thereof is configured to shoot the maxillary teeth, and a second camera 121 thereof is configured to shoot the mandibular teeth, so as to capture enough tooth surfaces at one time. Specifically, more inner surfaces and occlusal surfaces of the teeth in the oral cavity are shot at one time, reduce the number of shots and the need to adjust the angle, and improve shooting efficiency. The opening of the angle between the two cameras 121 is away from the main body 11 to prevent the main body 11 from blocking the two cameras 121, so that the light reflected back from the internal parts of the oral cavity is received by the two cameras 121.

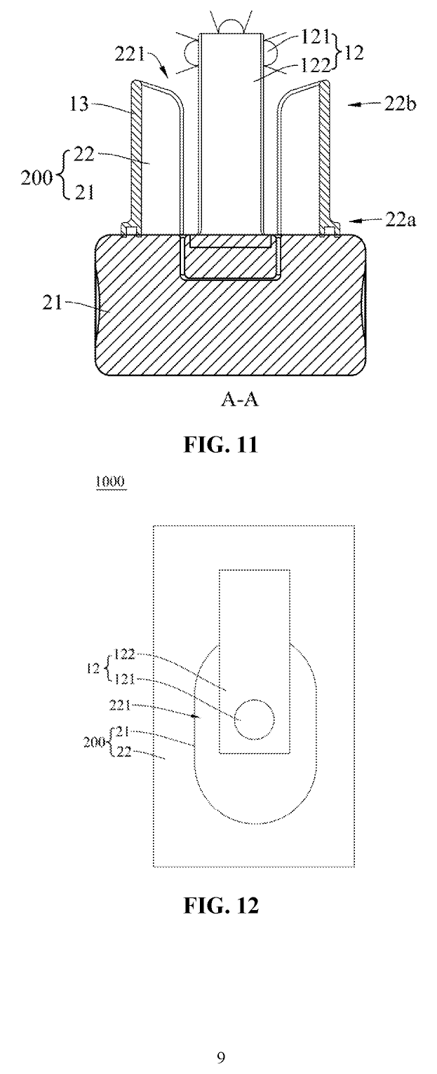

In some embodiments, as shown in FIG. 11, the camera module 12 includes three cameras 121 and the support piece 122. The support piece is retractable. The support piece 122 is connected to the main body 11, a first camera and a second camera 121 thereof are respectively disposed on two side surfaces of the support piece 122 in the first direction, and a third camera 121 thereof is disposed on an end surface of the support piece 122 in the axial direction of the imaging through hole 221.

In this way, in the first form, the third camera 121 disposed on an end surface of the support piece 122 in the axial direction of the imaging through hole 221 is exposed from the imaging through hole, so the outer surfaces of the teeth are shot. In the second form, the first camera 121 and the second camera 121 are respectively disposed on two side surfaces of the support piece 122 in the first direction, and the first camera 121 and the second camera 121 simultaneously shoot the image of the teeth located in the upper jaw and the image of the teeth located in the lower jaw, which ensures that the viewing angle and shooting angle of the two images are similar. Therefore, the image of the teeth located in the upper jaw and the image of the teeth located in the lower jaw have the same reference datum in space, which is convenient for the occlusal analysis, the evaluation of tooth alignment, and the identification of potential problems.

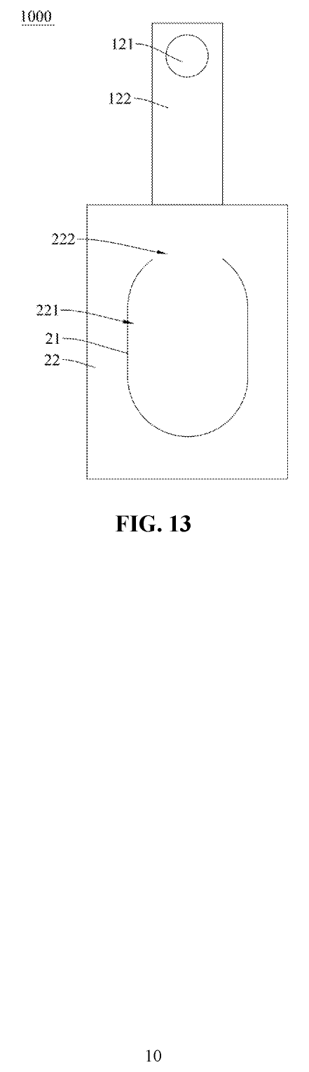

In some embodiments, as shown in FIGS. 12 and 13, the camera module 12 is detachably connected to the main body 11, and the camera module 12 is able to be connected to different positions of the main body 11. Specifically, when the support piece 122 is connected to a first position of the main body 11, the oral imaging device 1000 is in the first form, and the at least one camera 121 is aligned with the imaging through hole 221 of the mouth opener 200. When the support piece 122 is removed from the first position of the main body 11 and the support piece 122 is connected to the second position of the main body 11, the oral imaging device 1000 is in the second form. At this time, the support piece 122 and the at least one camera 121 protrude relative to the main body 11, so that the at least one camera 121 is allowed to be inserted into the oral cavity to take images of the internal parts in the oral cavity.

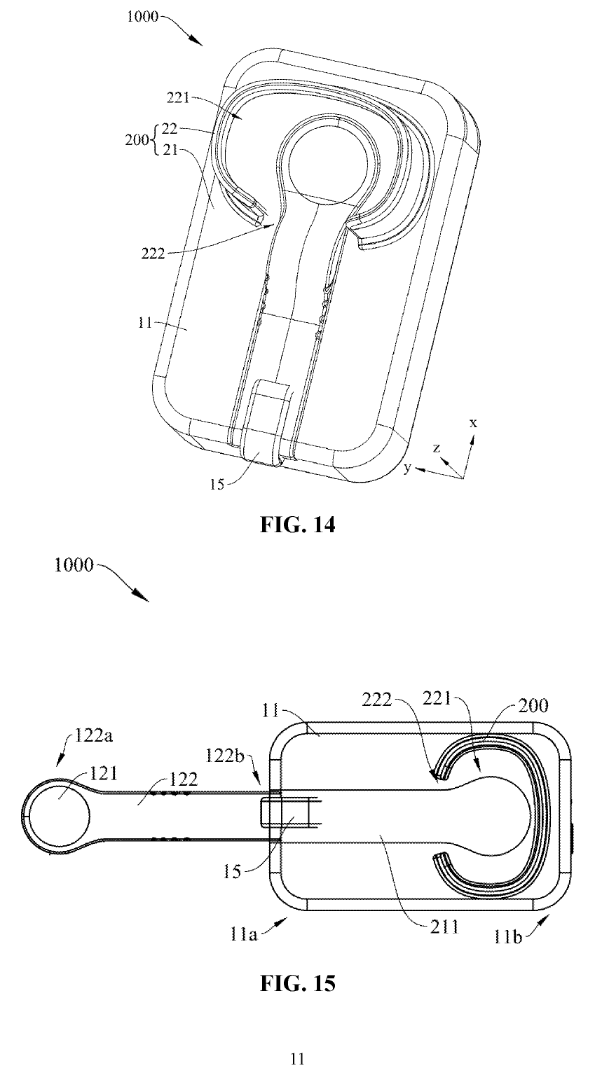

In some embodiments, as shown in FIGS. 14-15, a notch 222 is defined on a wall of the imaging through hole 221, and the notch 222 penetrates the end surface of the second end 22b of the mouth opener. The camera module 12 is rotatably connected to the main body 11, and the camera module 12 is rotatable to be located inside or outside the imaging through hole 221 through the notch 222. That is, the at least one camera 121 is allowed to insert into the imaging through hole 221 through the notch 222, and is folded on the main body 11. At this time, the oral imaging device 1000 is in the first form. When the support piece 122 is rotated to unfold relative to the main body 11, the at least one camera 121 rotates from the imaging through hole 221 to the outside of the imaging through hole 221, and the at least one camera 121 is staggered with the imaging through hole 221,. At this time, the oral imaging device 1000 is in the second form.

Specifically, the notch 222 is located in a rotation direction of the support piece 122, and notch 222 defines a space for the support piece 122 to rotate. In the first form, one end of the support piece 122 and the at least one camera 121 are located in the imaging through hole 221. When the oral imaging device 1000 is switched from the first form to the second form, the one end of the support piece 122 and the at least one camera 121 rotate from the imaging through hole 221 through the notch 222 to the outside of the imaging through hole 221. When the oral imaging device 1000 is switched from the second form to the first form, the one end of the support piece 122 and the at least one camera 121 rotate from the outside of the imaging through hole 221 through the notch 222 to an inside of the imaging through hole 221.

The support piece 122 of the camera module 12 is rotatably connected to the main body 11. When the one end of the support piece 122 and the at least one camera 121 are rotated to be located in the imaging through hole 221, the oral imaging device 1000 is in the first form, the support piece 122 is at least partially located in the imaging through hole 221, and the at least one camera 121 is aligned with the imaging through hole 221 of the mouth opener 200. When the support piece 122 is rotated to be located outside the imaging through hole 221, the oral imaging device 1000 is in the second form, the mouth opener 200 is connected to the main body 11, and the support piece 122 and the at least one camera 121 protrude relative to the main body 11.

For instance, the support piece 122 and the main body 11 are rotatably connected via a rotating shaft 15. An extension direction of the rotating shaft 15 is consistent with the width direction of the main body 11. The support piece 122 is rotatable on a plane where the length direction and the thickness direction are located. Specifically, when the one end of the support piece 122 and the at least one camera 121 are rotated to be located in the imaging through hole 221, the oral imaging device 1000 is in the first form. At this time, the support piece 122 and the at least one camera 121 are at least partially accommodated in the largest surface of the main body 11 and aligned with the imaging through hole 221 of the mouth opener 200, and the at least one camera 121 is aligned with the outer surfaces of the teeth through the imaging through hole 221 for shooting. The support piece 122 and the at least one camera 121 are at least partially located in the imaging through hole 221 of the mouth opener 200, which not only saves space, but also protects the support piece 122 and the at least one camera 121 from external damage, thereby extending their service life. When the oral imaging device 1000 needs to be switched from the first form to the second form, the support piece 122 rotates relative to the rotating shaft, stretches outward from the imaging through hole 221, gradually forms a certain angle with the main body 11, rotates to be roughly parallel to the main body 11, and finally protrudes relative to the main body 11. The support piece 122 is rotatable to be inserted into the oral cavity, and the at least one camera 121 faces the inner surfaces and the occlusal surfaces of the teeth for shooting.

The support piece 122 is rotatably connected to the main body 11 so that the support piece 122 is able to be folded on the main body 11 in the first form, thereby reducing the volume occupied by the support piece 122 and reducing the volume of the oral imaging device 1000, which is conducive to a miniaturization design of the oral imaging device 1000, and makes the oral imaging device 1000 easy to carry and store. The support piece 122 is at least partially located in the imaging through hole 221 of the mouth opener 200, and the at least one camera 121 is located in the imaging through hole 221 of the mouth opener 200, so the mouth opener further provides protection for the support piece 122 and the at least one camera 121 to avoid damage to the support piece 122 and the at least one camera 121 due to accidental collision or extrusion.

In some embodiments, in the first form, the second direction of the imaging through hole 221 is roughly parallel to the length direction of the oral imaging device 1000.

The short axis of the cross section of the imaging through hole 221 in the second direction is parallel to the length direction of the main body 11 and the oral imaging device 1000. When the user holds the main body 11 of the oral camera 100 and adjusts the position of the mouth opener 200, the direction of the imaging through hole 221 is adapted to a natural gesture of the user. For example, when the oral camera 100 is held vertically (that is, the length direction of the oral camera 100 is perpendicular to the horizontal plane), the long axis of the imaging through hole 221 enables the oral camera 100 to have greater freedom to adapt to such movement, so that the mouth opener 200 is smoothly moved along an arrangement direction of the teeth while maintaining a stable connection. The user can more easily slide the mouth opener 200 in an up-and-down direction or a left-and-right direction without worrying about the mouth opener 200 falling off the oral camera 100 due to lateral force.

When it is detected that the mouth opener 200 is held in the mouth of the user for oral examination, the first direction is parallel or approximately parallel to the coronal axis of the human body, and the length direction of the main body 11 of the oral camera 100 is parallel or approximately parallel to the vertical axis of the human body. Compared with a structure of the main body where the length direction of the main body 11 extends in the horizontal direction, a holding portion of the main body 11 is lengthened to facilitate user holding, so that the user is able to hold the main body 11 stably, and the user is able to stably hold the main body 11 when taking the oral images, and the operability and comfort of use are good.

In some embodiments, in the first form, a geometric center of the imaging through hole 221 is eccentrically disposed relative to a center of the oral imaging device 1000 in the length direction.

Since the opening and closing direction of the oral cavity is parallel to the vertical direction of the main body, the main body 11 extends in the vertical direction so that the center of the main body 11 is offset relative to a center of the mouth opener 200 in the vertical direction. In this way, the holding portion of the main body 11 is farther away from the oral cavity than the holding portion of the main body 11 extending in the horizontal direction, thereby reducing the probability of contact between the hand of the user and the oral cavity of the person being shot, and improving the hygiene level.

In some embodiments, in the first form, the at least one camera 121 has a rectangular FOV, and long sides of the rectangular FOV are parallel to the first direction.

Since the long axis of the imaging through hole 221 in the first direction is roughly parallel to the width direction of the dental arches when the mouth opener 200 is supported in the mouth, the long sides of the rectangular FOV are parallel to the first direction, and the long sides of the rectangular FOV are parallel to the width direction of the dental arches (in the left-and-right direction), so that the shooting range of the at least one camera 121 covers all of the dental arches, thereby facilitating the complete imaging of the dental arches.

In some embodiments, in the first form, on a projection plane perpendicular to the axis of the imaging through hole 221, the geometric center of the imaging through hole 221 does not coincide with a geometric center of the mouth opener 200, so that the holding portion of the main body 11 for the user to hold is farther away from the oral cavity than the holding portion of the main body 11 extending in the horizontal direction, which reduces the probability of the hand of the user contacting the oral cavity of the person being shot. Further, the hygiene level is improved.

In some embodiments, in the first form, the geometric center of the imaging through hole 221 does not coincide with the geometric center of the oral imaging device 1000, so that the holding portion of the oral imaging device 1000 for the user to hold is far away from the position of the imaging through hole 221, which reduces the probability of the hand of the user contacting the oral cavity of the person being shot. Further, the hygiene level is improved.

In some embodiments, the cross section of the imaging through hole 221 perpendicular to the axial direction has a largest dimension in the first direction. That is, when the mouth opener 200 is supported in the mouth, the dimension of the imaging through hole 221 in the direction of the coronal axis of the human body is the largest, and the mouth opener is supported between the lips of the person to be shot to open the mouth of the person. The opened mouth maintains a large space in the first direction x, so that the support piece 122 is allowed to be inserted into the mouth of the person to be shot for shooting.

In some embodiments, a shape of the imaging through hole 221 is selected from one of an elliptical hole, an oblong hole, and a rectangular hole, which enables the mouth of the person to be shot to be opened to define an opening similar to the shape of the imaging through hole 221, so that the at least one camera 121 is allowed to shoot the internal parts in the mouth.

In some embodiments, as shown in FIGS. 14-15, the mouth opener 200 includes a connecting portion 21 and an opening portion 22. The connecting portion 21 of the mouth opener 200 is connected to the oral camera 100. That is, the connecting portion 21 of the mouth opener 200 is connected to the main body 11 or the support piece 122. Two ends of the opening portion 22 are respectively the first end 22a and the second end 22b of the mouth opener 200. The first end 22a of the opening portion 22 is connected to the connecting portion 21 of the mouth opener 200, and the imaging through hole 221 is defined in the opening portion 22.

Specifically, the opening portion 22 is configured to open the lips. When the oral imaging device 1000 is in the first form, the user holds the opening portion 22 in his/her mouth, and the opening portion 22 abut against the gums and other parts to open the lips of the user. At least part of the opening portion 22 is located between the lips and the gums, which prevent the lips from fitting with the gums. At this time, at least the outer surfaces of the teeth on the gums correspond to the imaging through hole 221, and at least the outer surfaces of the teeth are located within the imaging range of the at least one camera 121. The at least one camera 121 is able to obtain the light signal of the part to be scanned (at least the outer surfaces of the teeth) through the imaging through hole 221 to generate the image or the video.

The connecting portion 21 is configured to connect the main body 11 and the opening portion 22. The connecting portion 21 is able to change a connection position of the opening portion 22 and the main body 11, so that the position of the opening portion 22 is flexibly adjusted relative to the main body 11.

In other words, the opening portion 22 of the mouth opener 200 is connected to the main body 11 of the oral camera 100 through the connecting portion 21, so that the opening portion 22 is indirectly connected to the main body 11. Compared with a solution where the opening portion 22 is directly connected to the main body 11, the connection position of the mouth opener 200 and the main body 11 is variable, and the position where the mouth opener 200 is connected to the main body 11 and the position of the opening portion 22 (the position of the imaging through hole 221) are decoupled. That is, the position where the mouth opener 200 is connected to the main body 11 is not related to the position of the opening portion 22. On the one hand, the imaging through hole 221 is within a range covered by the main body 11, or the imaging through hole 221 protrudes relative to the main body 11. That is, the imaging through hole 221 extends outside the range covered by the main body 11, and the position of the imaging through hole 221 of the opening portion 22 relative to the main body 11 is diverse.

In addition, the mouth opener 200 is connected to the main body 11 through the connecting portion 21, and there is no need to reserve a connection position of the mouth opener 200 on the support piece 122, so that the volume of the support piece 122 is controlled to be relatively small, so that the support piece 122 and the at least one camera 121 are easily inserted into the mouth.

In some embodiments, the connecting portion 21 of the mouth opener 200 is slidable on the main body 11, so that the oral imaging device 1000 is able to switch between the first form and the second form, and the opening portion 22 does not need to directly bear the friction during the sliding process, which reduces mechanical wear of the opening portion 22 and extends the service life of the opening portion 22.

In some embodiments, the isolating sleeve 13 covers at least part of the connecting portion 21. Specifically, the isolating sleeve 13 covers at least part of the connecting portion 21 in the first form. When the user uses the oral imaging device 1000, the isolating sleeve 13 prevents the oral cavity of the user from contacting at least part of the connecting portion 21, thereby effectively avoiding the hygienic problems that may be caused by different users cross-using the oral imaging device 1000.

In other words, the isolating sleeve 13 covers at least part of the connecting portion 21, thereby preventing the oral cavity of the user from contaminating the connecting portion 21, preventing cross-infection or bacterial transmission caused by the oral cavity of the user being in contact with the connecting portion 21, and improving the hygienic safety of the oral imaging device 1000.

In some embodiments, the opening portion 22 and the connecting portion 21 are an integrated structure. That is, the connecting portion 21 and the opening portion 22 are a one-piece structure, thereby improving a bonding strength between the connecting portion 21 and the opening portion 22, preventing the connecting portion 21 and the opening portion 22 from being separated during the operation of the mouth opener 200, and ensuring the stability and reliability of the operation of the mouth opener 200.

In other embodiments, the opening portion 22 and the connecting portion 21 are separate structures. That is, the connecting portion 21 and the opening portion 22 are two different structures. In one example, the opening portion 22 and the connecting portion 21 are detachably connected, and a detachable connection method thereof includes, but is not limited to a snap-fit connection or a threaded connection. In another example, the opening portion 22 is fixedly connected to the connecting portion 21, and a non-detachable connection method thereof includes but is not limited to bonding or welding.

In some embodiments, on a projection plane perpendicular to the optical axis of the at least one camera 121, the connection position of the connecting portion 21 and the main body 11 is at least partially offset relative to a connection position of the connecting portion 21 and the opening portion 22.

Specifically, the connection position of the connecting portion 21 of the mouth opener 200 and the main body 11 is at least partially offset relative to the connection position of the connecting portion 21 and the opening portion 22, so that the opening portion 22 is indirectly connected to the main body 11. Compared with a solution where the opening portion 22 is directly connected to the main body 11, the connection position of the mouth opener 200 and the main body 11 is variable, and the connection position of the mouth opener 200 and the main body 11 is decoupled from the position of the opening portion 22 (the position of the imaging through hole 221). That is, the connection position of the mouth opener 200 and the main body 11 is not related to the position of the opening portion 22, thereby the position of the at least one camera 121 is not limited, and the position of the at least one camera 121 is flexibly determined.

The connection position of the connecting portion 21 of the mouth opener 200 and the main body 11 is at least partially offset relative to the connection position of the connecting portion 21 and the opening portion 22, indicating that at least part of the connecting portion 21 is located between the opening portion 22 and the main body 11, so that a distance between the main body 11 and the opening portion 22 increases. When the outer surfaces of the teeth are shot in the first form, the distance between the hand of the user and the oral cavity increases, and the probability of the hand of the user contacting the oral cavity reduces. Therefore, the hygiene level is improved.

In some embodiments, in the length direction of the oral imaging device 1000, the center of the imaging through hole 221 does not coincide with the center of the connecting portion 21.

Specifically, in some embodiments, on the projection plane perpendicular to the extension direction of the imaging through hole 221, the geometric center of the imaging through hole 221 does not coincide with the geometric center of the connecting portion 21. The opening portion 22 of the mouth opener 200 is indirectly connected to the main body 11 through the connecting portion 21, rather than being directly connected to the main body 11. The connection position of the mouth opener 200 and the main body 11 changes, so that a projection of the opening portion 22 is located within the projection plane of the connecting portion 21. That is, the connection position of the mouth opener 200 and the main body 11 is not coupled with nor related to the position of the opening portion 22 (i.e., the position of the imaging through hole 221). Therefore, the position of the imaging through hole 221 is adjusted within the range covered by the main body 11, or the imaging through hole 221 is able to protrude relative to the main body 11 and extend beyond the range covered by the main body 11, so the position of the imaging through hole 221 is variable relative to the main body 11, allowing the user to flexibly adjust the shooting angle and position according to specific needs, and improving the adaptability and accuracy of imaging. Furthermore, the opening portion is indirectly connected to the main body, and the connecting portion 21 plays a certain buffering role, which reduces the impact of external forces on the opening portion 22, enhances the overall stability of the oral imaging device, and maintains a good working state even if the oral imaging device encounters a slight collision or movement. In addition, the disposable item (such as the isolating sleeve 13) is easier to be sleeved on the mouth opener 200. Because the geometric center of the imaging through hole 221 does not coincide with the connecting portion 21, the disposable item is smoothly sleeved on the mouth opener, which simplifies operation steps.

The geometric center of the imaging through hole 221 does not coincide with the geometric center of the connecting portion 21, and the portion of the connecting portion 21 that is not located around the geometric center of the imaging through hole 221 is configured to connect with the main body 11. On the one hand, the position of the imaging through hole 221 relative to the main body 11 is flexibly adjusted, and on the other hand, the position of the imaging through hole 221 does not occupy the space of the main body 11, and the holding portion of the main body for the user to hold is sufficient, thereby improving the holding stability of the use..

In some embodiments, the connecting portion 21 is connected to at least one of the main body 11 and the camera module 12, thereby improving the use stability of the mouth opener 200 in the first form.

In some embodiments, the main body 11 extends along the length direction. The main body 11 includes a connecting surface 111 in the length direction thereof, and the main body 11 further includes a first peripheral side surface 112 connected to the connecting surface 111. The support piece 122 is connected to the connecting surface 111 of the main body 11. The support piece 122 is disposed along the length direction of the main body 11. The at least one camera 121 is disposed at the one end of the support piece 122 away from the main body 11.

In some embodiments, the connecting portion 21 of the mouth opener 200 is connected to the connecting surface 111 (end surface) of the main body 11, rather than occupying a large surface of the main body 11 (the peripheral side surface of the main body 11). The so-called "large surface" refers to a main surface on the main body 11 for mounting or connecting other components, and the large surface is commonly the front surface or the side surface of the main body 11. By configuring the connection position of the mouth opener 200 on the end surface of the main body 11 instead of the large surface, the peripheral side surface of the main body 11maintains a simple and flat design, thereby providing a comfortable grip for the user, while reducing the overall volume of the main body 11. Therefore, it is easy for the user to hold and operate the oral imaging device 1000 for a long time.

In addition, the connecting portion 21 of the mouth opener 200 is connected to the connecting surface 111 (end surface) of the main body 11, rather than connecting to the peripheral side surface of the main body 11, which increases the distance between the main body 11 and the opening portion 22. When shooting the outer surfaces of the teeth in the first form, the distance between the hand of the user and the mouth increases, thereby reducing the probability of the hand of the user contacting the mouth. Therefore, the hygiene level of the device is thus improved.

In some embodiments, as shown in FIGS. 1-3, when the connecting portion 21 is connected to the connecting surface 111 of the main body 11, a fitting groove 211 is defined in the connecting portion 21. The fitting groove 211 is matched with the support piece 122, and the fitting groove 211 extends along the length direction of the main body 11. The support piece 122 is engaged with the fitting groove 211 along the length direction of the main body 11.

Specifically, the fitting groove 211 is configured to accommodate at least part of the support piece 122 in the first form. A shape of a cross section of the fitting groove 211 is rectangular, circular etc. Optionally, a shape and a size of the fitting groove 211 are matched with an outer contour of the support piece 122, thereby ensuring that an inner wall of the support piece 122 and the fitting groove 211 form a clearance fit, so the support piece 122 is well positioned and supported by the fitting groove 211, thereby ensuring the stable mounting of the support piece 122, making it difficult for the support piece 122 to shift, and avoiding displacement or loosening of the support piece. The fitting groove 211 further plays a positioning role, which facilitates the mounting of the support piece 122.

In some embodiments, the fitting groove 211 of the connecting portion 21 is communicated with the imaging through hole 221, and the fitting groove 211 is a slot recessed from a surface of the connecting portion 21 on one side where the opening portion 22 is disposed. In this way, when the support piece 122 is plugged and matched with the connecting portion 21, the support piece 122 is embedded in the fitting groove 211, and the at least one camera 121 on the support piece 122 is allowed to be extended to the position corresponding to the imaging through hole 221 through the fitting groove 211. Therefore, the at least one camera 121 is able to shoot the internal parts of the oral cavity of the person being shot through the imaging through hole 221.

In addition, the fitting groove 211 is only defined in the surface of the opening portion 22 on the connecting portion 21. The fitting groove 211 limits the connection position and the connection direction of the mouth opener 200 and the support piece 122, so that the connection position and the connection direction of the mouth opener 200 are unique, which has a good foolproof effect and avoids incorrect mounting.

In some embodiments, in the second direction of the imaging through hole 221, a length of the fitting groove 211 is greater than half a length of the connecting portion 21. In this way, the support piece 122 and the connecting portion 21 have sufficient lengths for plug-in matching, so that the mouth opener 200 is firmly connected to the oral camera 100, and the mouth opener 200 is prevented from being separated from the oral camera 100 during the shooting process.

In some embodiments, one end, close to the main body 11 in the second direction of the imaging through hole 221, of the connecting portion 21 is connected to the main body 11. Since the imaging through hole 221 has the long axis in the first direction and the short axis in the second direction, the one end of the connecting portion 21 close to the main body 11 in the second direction is connected to the main body 11, so that the oral camera 100 is movable in a long axis direction (i.e., the first direction) of the imaging through hole 221. That is, the oral camera has a large adjustment space and is adapted to the shape of the dental arches. Therefore, the oral camera is allowed to shoot different positions of the dental arches in the oral cavity.

In some embodiments, at least part of the connecting portion 21 is flush with the surface of the main body 11, which makes an appearance of the oral imaging device 1000 concise and beautiful, and at the same time prevents the connecting portion 21 from protruding from the surface of the main body 11 and scratching the mouth or the face of the user, thereby improving the comfort and safety of the oral imaging device 1000 during use.

In some embodiments, when the connecting portion 21 is connected to the connecting surface 111 of the main body; at least one first magnetic piece 212 is disposed on the connecting surface 111 of the main body, at least one second magnetic piece 1111 is disposed on the surface of the connecting portion 21 facing the connecting surface 111 of the main body, and the connecting surface 111 of the main body and the connecting portion 21 are detachably connected through the at least one first magnetic piece 212 and the at least one second magnetic piece 1111.

Specifically, the at least one first magnetic piece 212 includes one or more first magnetic pieces 212, which are not limited in the present disclosure. The at least one second magnetic piece 1111includes one or more magnetic pieces 212 which are not limited in the present disclosure. The number of the one or more first magnetic pieces 212 may be the same or different from the number of the one or more second magnetic pieces 1111. When the mouth opener 200 needs to be connected to the main body 11, the one or more first magnetic pieces 212 are magnetically connected to the one or more second magnetic pieces 1111, thereby completing the connection between the mouth opener 200 and the main body 11. When the mouth opener 200 needs to be separated from the main body 11, it is only necessary to overcome the magnetic force and manually separate the mouth opener 200 from the main body 11.

The one or more first magnetic pieces 212 and the one or more second magnetic pieces 1111 are magnetically connected by the magnetic force. A magnetic connection thereof is stable and reliable, so the mouth opener 200 is effectively prevented from loosening or falling off due to external force during use. In addition, the magnetic connection further has a certain self-alignment function, which ensures that the mouth opener 200 and the main body 11 are quickly aligned when connected, thereby improving the efficiency and accuracy of assembly of the oral imaging device 1000.

In some embodiments, the connecting portion 21 is connected to the first peripheral side surface 112 of the main body 11. It is understood that a contour of the first peripheral side surface 112 of the main body 11 at least includes the long sides of the main body 11 in the length direction, and the first peripheral side surface 112 of the main body 11 is a relatively flat surface of the main body 11. The connecting portion 21 is connected to the first peripheral side surface 112 of the main body 11, so a connection area between the connecting portion 21 and the main body is relatively large and the connecting portion 21 and the main body 11 are stably connected. Moreover, the connecting portion 21 is connected to the first peripheral side surface 112 of the main body 11, which limits a movement of the mouth opener 200 in the thickness direction of the main body 11, and ensures that the mouth opener 200 and the main body 11 do not move.

In addition, when shooting the oral cavity in the first form, the user commonly holds the first peripheral side surface 112 of the main body 11, and the connecting portion 21 is connected to the first peripheral side surface 112 of the main body 11, and the connecting portion 21 is held by the user, thereby further improving the stability of the mouth opener 200 on the main body 11.

In some embodiments, when the connecting portion 21 is connected to the first peripheral side surface 112 of the main body 11, the connecting portion 21 covers the support piece 122, so that the support piece 122 is prevented from contacting the internal parts of the oral cavity of the person being shot during the shooting process. When the same oral camera 100 is used to shoot different users, cross contact or contamination is prevented. At the same time, the connecting portion 21 protects the at least one camera 121 and the support piece 122.

In some embodiments, the connecting portion 21 is covered on the surface of the main body. The connecting portion 21 covers the surface of the main body 11, which prevents the surface of the main body 11 from contacting the lips of the person being shot. In this way, the hygiene level is improved, so that the same oral camera 100 is allowed to shoot different users for a period of time without the need to disinfect after each shooting.

In some embodiments, as shown in FIGS. 1-3, the connecting portion 21 includes a first surface 21a and a second surface 21b opposite to the first surface 21a in the thickness direction of the main body 11. The opening portion 22 is disposed on the first surface 21a of the connecting portion 21. When the connecting portion 21 is connected to the first peripheral side surface 112 of the main body 11, a peripheral edge of the connecting portion 21 is bent so that the connecting portion 21 is in a shell structure. The connecting portion 21 is sleeved on the outer periphery of the main body 11. The second surface 21b of the connecting portion 21 is connected to the first peripheral side surface 112 of the main body 11. The shape of the connecting portion 21 adopts the shell structure, and is able to be sleeved on the outer periphery of the main body 11 like a mobile phone case sleeved on a mobile phone. The shell structure is simple, and a larger oral imaging area is obtained. The shell structure has good connection stability, and the at least one camera 121 is allowed to have a large shooting distance by being disposed away from the opening portion or the at least one camera 121 is allowed to have a large shooting distance without being inserted into the opening portion. The peripheral edge of the connecting portion 21 is bent and connected to an edge of the main body 11 to be sleeved on the main body 11. At the same time, the connecting portion 21 covers the outer periphery of the main body 11, which prevents the main body 11 from contacting the lips of the user, thus further improving the hygiene level. Therefore, the same oral camera 100 is allowed to shoot different users over a period of time without the need for disinfection after each shooting.

In some embodiments, at least part of the connecting portion 21 is made of magnetic material, and the second surface 21b of the connecting portion 21 is magnetically connected to the first peripheral side surface 112 of the main body 11. Specifically, the connecting portion 21 is magnetically connected to the first peripheral side surface 112 of the main body 11. On the one hand, a magnetic connection thereof is simple and convenient. On the other hand, a connection area of the connecting portion 21 and the first peripheral side surface 112 of the main body 11 is large, and the generated magnetic force is large, which increases the connection stability of the connecting portion 21 and the main body 11.

In some embodiments, the at least one camera 121 includes a wide-angle camera 121, and the FOV of the at least one camera 121 is not less than 140, so that when the support piece 122 is located between the two dental arches, the at least one camera 121 is at least configured to capture the image of the occlusal surfaces of the teeth on one of the two dental arches.

If the FOV of the at least one camera 121 is less than 140, when shooting the part to be scanned in the oral cavity, the imaging range of the at least one camera 121 is limited, and the at least one camera 121 is impossible to capture enough oral images at one time, which results in the need to adjust the shooting angle or shooting position multiple times to obtain a complete oral image, which increases the time and complexity of shooting and reduces the shooting efficiency.

The FOV of the at least one camera 121 is not less than 140, which provides a wide shooting range, and has both comprehensiveness and efficiency in shooting. When the support piece 122 is inserted into the mouth of the user, the at least one camera 121 has a large imaging range and is able to capture the image including more teeth in a single shot. The image includes the occlusal surfaces, the inner surfaces and the outer surfaces of the teeth, and or the image includes multiple teeth, or the image includes two complete dental arches, thereby reducing the number of shots and improving shooting efficiency.

In other words, when the FOV of the at least one camera 121 is not less than 140°, and when the mouth is opened to a moderate size and the at least one camera 121 is inserted into the mouth, the at least one camera 121 is able to shoot one of the dental arches (i.e., the target dental arch, which is the to-be-shot dental arch) with the larger imaging range. For example, a shooting area of the at least one camera 121 may cover the entire target dental arch, so that a more complete or wider image of the target dental arch is obtained. Thus, a panoramic image of the target dental arch is obtained in a single shot in the mouth, without the need for the reflector, and the operation process is relatively simple and easy to operate. Therefore, the user is able to operate and check the oral cavity by himself/herself.

When a distance between the at least one camera 121 and an object (such as the upper dental arch) is set to 20 mm, a short side length of a captured image is ≥60 mm (diagonal ≥100 mm), and the FOV of the at least one camera 121 must be not less than 140°. Therefore, in some embodiments, the FOV of the at least one camera 121 is greater than or equal to 170, so that the shooting area of the at least one camera 121 covers the entire target dental arch and captures the entire occlusal surfaces of the teeth while further reducing the opening degree of the mouth, which is conducive to capturing the panoramic image that is more in line with the standard of dentist shooting.

In other words, when the FOV of the at least one camera 121 is not less than 140°, and when the mouth is opened to a moderate size and the at least one camera 121 is inserted into the mouth, the at least one camera 121 is able to shoot the target dental arch with the large imaging range. For example, a shooting area of the at least one camera 121 may cover the entire target dental arch, so that a complete or wide image of the target dental arch is obtained. Thus, a panoramic image of the target dental arch is obtained in a single shot in the mouth, without the need for the reflector, and the operation process is relatively simple and easy to operate. Therefore, the user is able to operate and check the oral cavity by himself/herself.

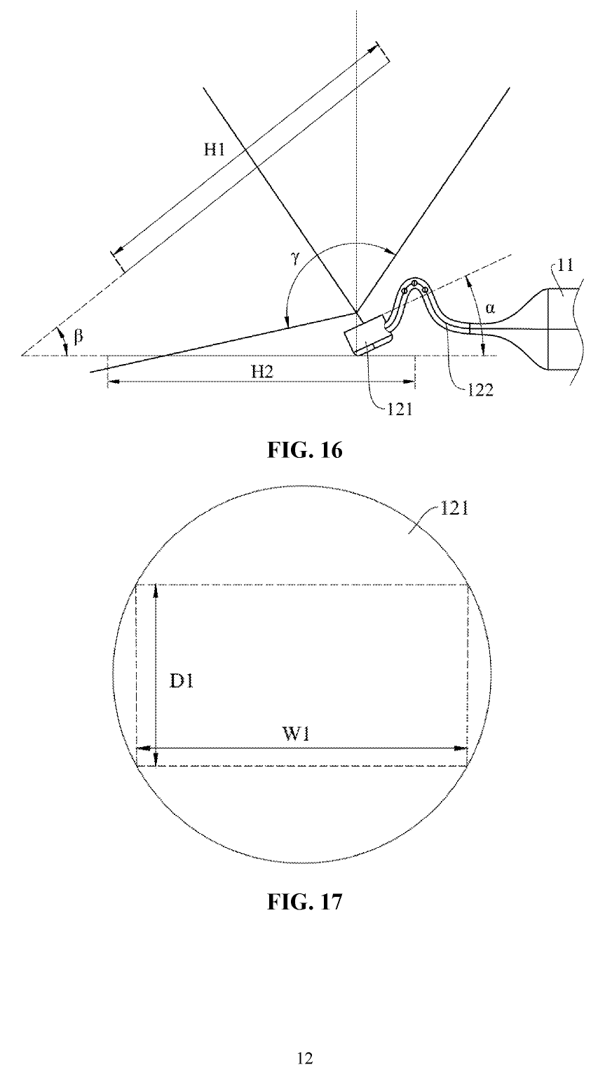

As shown in FIG. 16, taking an example that an average length of any of the dental arches is H1=H2=50 mm for further illustration, under a condition that the FOV angle γ of the at least one camera 121 is not less than 140°, when a light incident surface of the at least one camera 121 is parallel to the upper dental arch and located at the midline of the dental arches, it is easiest to obtain an image of a complete upper dental arch. Assuming an opening angle of the user is β, and an inclined angle of the at least one camera 121 is α, then β=α. Combined with a clinical mouth opening range of 30°-45° for adults and 20°-30° for children, the inclination angle α of the at least one camera 121 is 25°-35°. In conjunction with a fulcrum fine-tuning function of an abutting portion 1222, the oral imaging device 1000 of the present disclosure is able to capture an image covering the complete occlusal surfaces and partial inner surfaces of the teeth on the dental arches in a single shot.

In some embodiments, the at least one camera 121 defines a rectangular FOV (such as a dotted box in FIG. 17), and the short sides of the rectangular FOV are parallel to the length direction of the support piece 122.

The rectangular FOV means that the shooting area captured by the at least one camera 121 is rectangular in shape. The rectangular FOV is divided into a horizontal FOV (HFoV) and a vertical FOV (VFoV). The horizontal FOV determines the maximum angle that is seen on left and right sides of the at least one camera 121, while the vertical FOV determines the maximum angle that is seen on upper and lower sides of the at least one camera 121.

The FOV of the at least one camera 121 is rectangular as a whole, and the maximum range of the FOV of the at least one camera 121 corresponds to the diagonal of the at least one camera 121. The short sides of the rectangular FOV are parallel to the length direction of the support piece 122 (i.e., the extension direction of the support piece 122 away from the main body 11), and the vertical FOV of the at least one camera 121 extends along the length direction of the support piece 122, so that the at least one camera 121 covers more occlusal surfaces or inner surfaces of the teeth, thereby improving the FOV utilization of the at least one camera 121. The user is able to obtain a better viewing angle by adjusting the angle of the support piece 122.

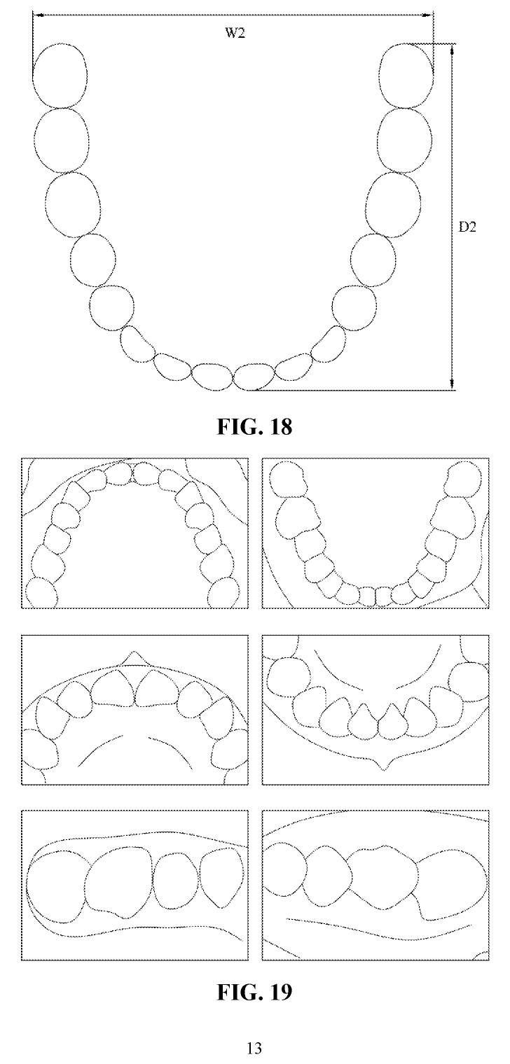

As shown in FIG. 17, the short sides (D1) of the rectangular FOV of the at least one camera 121 are parallel to the length direction of the support piece 122, and the long sides (W1) of the rectangular FOV of the at least one camera 121 are parallel to the width direction of the support piece 122. A ratio of W1 to D1 is 4:3 or 16:9, which is not limited thereto. When the support piece 122 is placed against the dental arches, an angle between the optical axis of the at least one camera 121 and the occlusal surfaces of the teeth is 60°-90° (to obtain a better shooting angle). As shown in FIG. 18, a relationship between a depth D2 of any one of the dental arches (a distance from the molar to the incisor edge) and a width W2 of any one of the dental arches (the distance between molar edges on two sides) determines that the short sides of the rectangular FOV must be parallel to a depth direction of the oral cavity, and the long sides must be parallel to the width direction of the dental arches (left and right direction), so as to achieve complete imaging of the dental arches.

Therefore, the short sides of the rectangular FOV of the at least one camera 121 are parallel to the length direction of the support piece 122, so the FOV is more effectively utilized, and the at least one camera 121 is compatible with a small shooting distance at a specific FOV angle, or at least one camera 121 is able to capture a larger area of the dental arches at a specific shooting distance, which is more conducive to capturing the panoramic image of the occlusal surfaces.

It should be noted that when shooting, the optical axis of the at least one camera 121 should be as perpendicular to the occlusal surfaces of the teeth as possible, or the optical axis of the at least one camera 121 must be maintained between 60° and 90°, so as to avoid pits and fissures of the occlusal surfaces of the teeth being blocked due to excessive shooting angles. Therefore, the panoramic image of the occlusal surfaces of the teeth.is obtained.

FIG. 19 is a schematic diagram of commonly oral images in clinical practice, which includes the images of complete dental arches, images of front teeth partial images of molar occlusal surfaces, etc.. Through the configurations of the first abutting portion 1222a, a second abutting portion 1222b and a third abutting portion 121c, the dentist is able to adjust the angle and the position of the at least one camera 121 in the oral cavity to obtain these images.

In some embodiments, on the projection plane perpendicular to the length direction of the main body 11, an area of the projection plane of the main body 11 is greater than the area of a projection plane of the support piece 122. On the one hand, when the connecting portion 21 of the mouth opener 200 is connected to the connecting surface 111 of the main body 11 in its length direction, the mouth opener 200 can obtain a larger connection area, thereby improving the stability of the connection. On the other hand, the volume of the support piece 122 is made relatively small, which is convenient for entry and movement of the support piece 122 in the oral cavity, making the movement of the support piece 122 in the oral cavity more flexible. Therefore, a total volume of the at least one camera 121 and the support piece 122 is not too large to reduce a distance between the at least one camera 121 and the teeth to be shot, so a shooting area of the teeth obtained by the at least one camera 121 is not limited.

In some embodiments, the thickness of the position where the at least one camera 121 is disposed is not greater than 15 mm.

If the thickness of the position where the at least one camera 121 is located is greater than 15 mm, the distance between the at least one camera 121 and the teeth to be shot is limited, so the shooting area of the teeth obtained by the at least one camera 121 is limited.

Therefore, the thickness of the position where the at least one camera 121 is located is not greater than 15 mm, which prevents the at least one camera 121 from being too close to the teeth to be shot, so that the teeth to be shot are controlled within a suitable shooting range of the at least one camera 121 to obtain a wide shooting range and cover more tooth surfaces. For example, the at least one camera 121 is placed below or above a highest point of the first dental arch 301 in the mouth of the user, thereby increasing the distance between the at least one camera 121 and the second dental arch 302, achieving a larger range or wider angle shooting of the second dental arch 302, and obtaining a more complete or wider image of the second dental arch 302.

In some embodiments, the support piece 122 includes a third end surface 122c, a fourth end surface 122d, and a second peripheral side surface 122e. The third end surface 122c and the fourth end surface 122d of the support piece are disposed opposite to each other in the length direction of the main body 11. The third end surface 122c of the support piece is connected to the main body 11. The second peripheral side surface 122e of the support piece is connected to the third end surface 122c and the fourth end surface 122d. The at least one camera 121 is disposed on the second peripheral side surface 122e of the support piece.

Specifically, the fourth end surface 122d of the support piece is a position farthest from the third end surface 122c on the support piece 122. For instance, the fourth end surface 122d of the support piece is perpendicular to the length direction of the main body 11. It is understood that the dimension of the main body 11 in the length direction is the maximum dimension of the main body 11 in the three directions. In an ideal case where the user uses the oral camera 100, the main body 11 is held by the user in a roughly vertical holding state. Vertical holding means that when the main body 11 is held, the length direction of the main body 11 is roughly perpendicular to the horizontal plane, and the width direction and thickness direction of the main body are roughly parallel to the horizontal plane. If the at least one camera 121 is disposed on the fourth end surface 122d of the support piece, then in the first form, the at least one camera 121 is difficult to align with the imaging through hole, and the user needs to raise the arm and maintain a certain angle when using the oral camera 100, so that the fourth end surface 122d of the support piece of the main body 11 faces the interior of the oral cavity for shooting. This posture causes the largest surface of the main body 11 to face downward, and the user needs to continue to exert force to maintain the position of the arm and the main body 11, which is easy to cause fatigue and difficult to maintain a stable shooting state.

When the at least one camera 121 is disposed on the second peripheral side surface 122e, the user needs to hold the main body 11 vertically, so that the length direction of the main body 11 is roughly perpendicular to the horizontal plane, and the width direction and thickness direction are roughly parallel to the horizontal plane. At this time, the second peripheral side surface 122e of the support piece where the at least one camera 121 is disposed naturally aligns with the oral cavity of the user, and the user is able to shoot without raising his/her arm or adjusting the angle of the main body 11. The user holds the main body 11 comfortably, which reduces arm fatigue and helps the at least one camera 121 to aim at the teeth to be shot more stably, so as to obtain a clearer and more stable oral image.

The second peripheral side surface 122e of the support piece 12 includes first sub-side surfaces 122e1 in the thickness direction of the support piece 122 and second sub-side surfaces 122e2 in the width direction of the support piece 122. The first sub-side surfaces 122e1 are opposite to each other. The second sub-side surfaces 122e2 are opposite to each other.

In some embodiments, the angle between the optical axis of the at least one camera 121 and the length direction of the support piece 122 is not less than 40° and not greater than 90°.

If the angle between the optical axis of the at least one camera 121 and the length direction of the support 122 is less than 40°, the optical axis of the at least one camera 121 needs to be adjusted at a greater angle when aligned with the occlusal surfaces of the teeth, resulting in an inaccurate shooting angle and difficulty in clearly obtaining the details of the occlusal surfaces of the teeth and information on some inner surfaces of the teeth. If the angle between the optical axis of the at least one camera 121 and the length direction of the support 122 is greater than 90°, the shooting angle of the at least one camera 121 is too tilted. When shooting, the optical axis of the at least one camera 121 may fail to accurately align with the occlusal surfaces of the teeth, and the shooting angle on the inner surfaces of the teeth may be too offset, resulting in blurred or incomplete images of the inner surfaces of the teeth. Moreover, an overly tilted shooting angle may further affect the contrast and clarity of the image, affecting the comprehensiveness of tooth shooting.

The angle between the optical axis of the at least one camera 121 and the length direction of the support piece 122 is not less than 40° and not greater than 90°, which provides a moderate angle fine-tuning function to ensure that the at least one camera 121 faces the occlusal surfaces of the teeth. In some cases, the at least one camera 121 shoots both the occlusal surfaces and the inner surfaces of the teeth, and the shooting angle is not excessively inclined to ensure that the at least one camera 121 faces the occlusal surfaces and the inner surfaces of the teeth when shooting.

In some embodiments, in the length direction of the support piece 122 away from the main body 11, the optical axis of the at least one camera 121 is inclined away from the main body 11 from the surface where the at least one camera 121 is located.

With such a configuration, when the user opens his mouth to the moderate size and the at least one camera 121 is placed in the mouth, the angle between the optical axis of the at least one camera 121 and the occlusal surfaces of the teeth is approximately 90°. That is, the shooting surface of the at least one camera 121 is made flush with the occlusal surfaces of the teeth as much as possible, so as to avoid the pits and the fissures of the occlusal surfaces of the teeth being blocked due to excessive shooting angles. Therefore, the panoramic image of the occlusal surfaces of the teeth.is obtained.

In some embodiments, the angle between the optical axis of the at least one camera 121 and the length direction of the support piece 122 is not less than 40° and not greater than 70°.

When the user opens his mouth to the moderate size and the at least one camera 121 is placed in the mouth, it is ensured that the at least one camera 121 is aligned with the occlusal surfaces of the teeth. In some cases, the at least one camera 121 shoots both the occlusal surfaces and the inner surfaces of the teeth, and the shooting angle is not excessively inclined to ensure that the at least one camera 121 faces the occlusal surfaces and the inner surfaces of the teeth when shooting. In addition, by limiting the angle between the optical axis of the at least one camera 121 and the length direction of the support piece 122 within a range of 40°-70°, the optical axis of the at least one camera 121 is inclined relative to the support piece 122, which helps the user find the shooting angle more quickly, reduces a bending angle of the wrist of the user when shooting, and improves comfort.

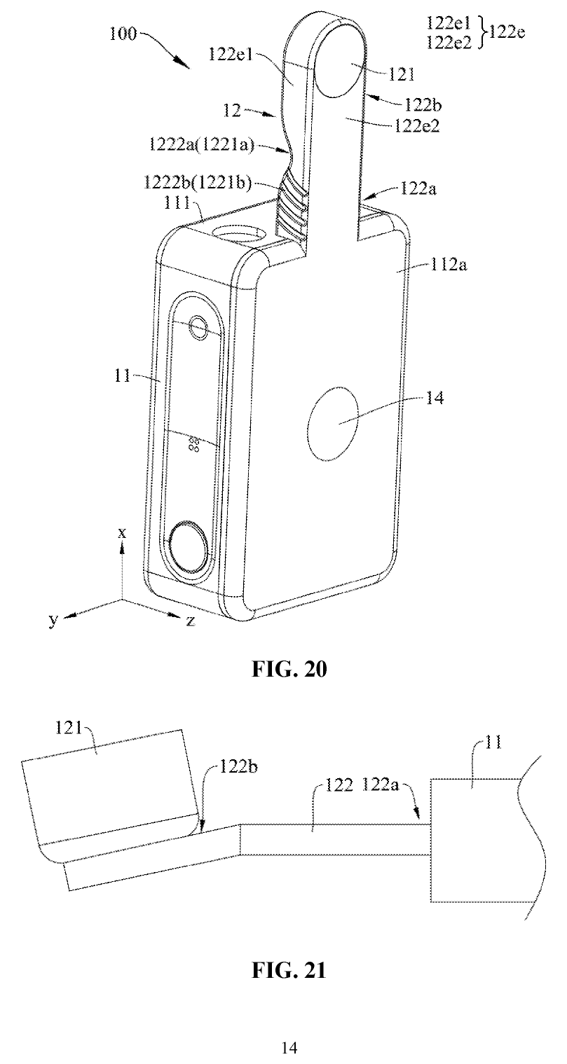

In some embodiments, as shown in FIGS. 20-21, the support piece 122 is bent along the thickness direction thereof, so that the optical axis of the at least one camera 121 is inclined relative to the length direction of the support piece 122. When the user opens his mouth to the moderate size and the at least one camera 121 is placed in the mouth, the angle between the optical axis of the at least one camera 121 and the occlusal surfaces of the teeth is approximately 90°. That is, the shooting surface of the at least one camera 121 is made flush with the occlusal surfaces of the teeth as much as possible, so as to avoid the pits and the fissures of the occlusal surfaces of the teeth being blocked due to excessive shooting angles. Therefore, the panoramic image of the occlusal surfaces of the teeth.is obtained.

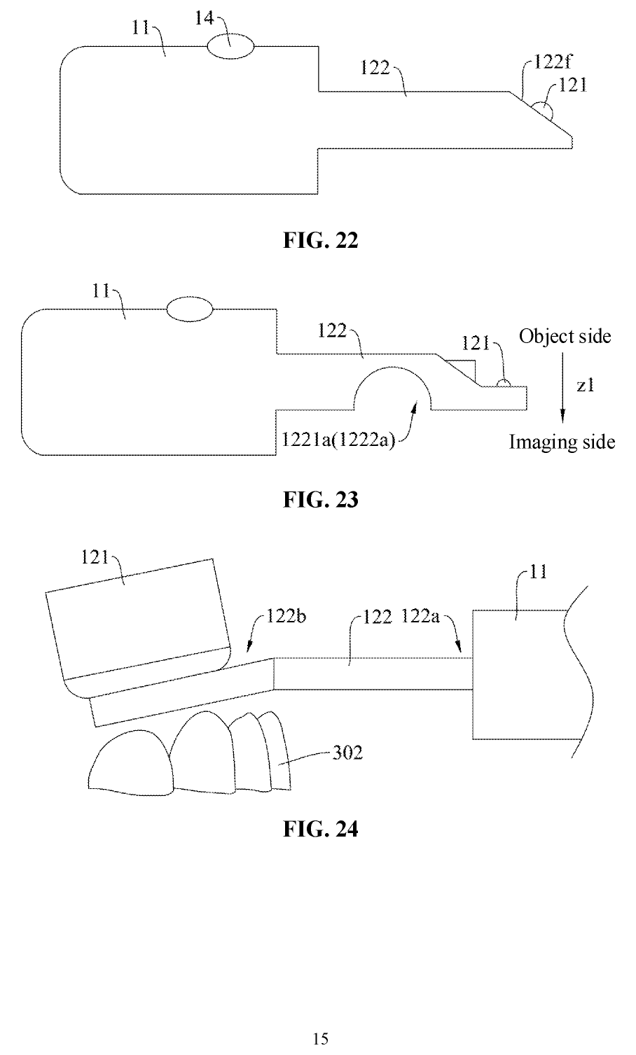

In some embodiments, as shown in FIG. 22, the support piece 122 includes a mounting surface 122f configured to mount the at least one camera 121. The mounting surface 122f of the support piece 122 is inclined relative to the length direction of the support piece 122. The thickness of the support piece 122 decreases in a direction from the support piece 122 to the main body 11. The at least one camera 121 is disposed on the mounting surface 122f of the support piece 122, so that the optical axis of the at least one camera 121 is inclined relative to the length direction of the support piece 122. When the user opens his mouth to the moderate size and the at least one camera 121 is placed in the mouth, the angle between the optical axis of the at least one camera 121 and the occlusal surfaces of the teeth is approximately 90°. That is, the shooting surface of the at least one camera 121 is made flush with the occlusal surfaces of the teeth as much as possible, so as to avoid the pits and the fissures of the occlusal surfaces of the teeth being blocked due to excessive shooting angles. Therefore, the panoramic image of the occlusal surfaces of the teeth.is obtained.

In some embodiments, as shown in FIG. 20, the first peripheral side surface 112 of the main body 11 includes a first side surface 112a and a second side surface 112b. The first side surface 112a of the main body 11 has a same orientation as the at least one camera 121, and the second side surface 112b is opposite to the at least one camera 121. At least one of a portion of the first side surface 112a of the main body 11 and a portion of the second side surface 112b of the main body 11 is a flat surface.

At least one of the portions of the first side surface 112a of the main body 11 and the portion of the second side surface 112b of the main body 11 is the flat surface. On the one hand, and since the shooting direction of the at least one camera 121 is parallel to the flat surface, the user is able to recognize the flat surface by touching the main body 11 through the fingers, thereby determining the direction and angle of the at least one camera 121, which is conducive to capturing a desired target image. Further, the main body 11 is prevented from rolling when being held, so that the user is able to hold the main body 11 stably. That is, the flat surface improves the stability of the user holding the main body 11 and further facilitates capturing the desired target image.

In addition, since the at least one camera 121 is not directly disposed on the main body 11, but is disposed on the main body 11 through the support piece 122, the main body 11 has a larger holding area, which is convenient for the user to hold, so that the user can well hold the main body 11, thereby making the oral camera 100 operable and comfortable to use. When taking the oral images, the hand of the user is away from the oral cavity, thereby reducing the probability of the hand of the user contacting the oral cavity and improving the hygiene level.

Considering that when using the at least one camera 121 to take images, the user needs to control the oral camera to shoot. Based on this, in some embodiments, the oral camera 100 further includes at least one button 14. The at least one button 14 is disposed on the main body and is disposed on at least one of a same side and an opposite side of the at least one camera 121 disposed on the support piece. That is, the at least one button 14 is disposed on at least one of the first side surface 112a and the second side surface 112b of the main body, so that the fingers of the user are allowed to control the at least one button 14 when holding the first side surface 112a and the second side surface 112b of the main body 11, thereby making the oral camera 100 operable and comfortable to use. When taking the oral images, the hand of the user is away from the oral cavity, thereby reducing the probability of the hand of the user contacting the oral cavity and improving the hygiene level.

In addition, at least one of the portion of the first side surface 112a of the main body 11 and the portion of the second side surface 112b of the main body 11 is the flat surface, and the at least one button 14 of the oral camera 100 is disposed on the flat surface of the main body. Therefore, the user is allowed to press the at least one button 14 steadily and reliably, avoiding problems such as swinging and falling of the oral camera 100 caused by uneven force.

Optionally, the at least one button 14 includes, but is not limited to a physical button 14, a virtual button 14 (such as a touch button 14), etc.

Of course, in other embodiments, the at least one button 14 may be disposed on at least one of the two opposite sides of the main body 11 along the width direction, which is not limited thereto.

In some embodiments, at least one of the first side surface 112a and the second side surface 112b of the main body 11 is the largest surfaces of the main body 11, and at least one of the portion of the first side surface 112a of the main body 11 and the portion of the second side surface 112b of the main body 11 is the flat surface, so that at least one of the first side surface 112a and the second side surface 112b of the main body 11 provides sufficient space for the fingers of the user to hold. Further, such a configuration conforms to a gripping posture of the hand, so as to avoid problems such as displacement and falling of the oral camera 100 during use due to small gripping space or unstable gripping position.

In some embodiments, in the direction perpendicular to the length direction of the main body 11, at least part of the cross sections of the main body 11 is greater than the cross sections of the support piece 122. The length direction of the main body 11 and the length direction of the support piece 122 are in the same direction.

In some embodiments, the width of at least a portion of the main body 11 is greater than the width of the support piece 122, and the thickness of at least a portion of the main body 11 is greater than the thickness of the support piece 122. Since the width of the support piece 122 determines the opening degree of the mouth of the user, by making the width of at least the portion of the main body 11 greater than the width of the support piece 122, the support piece 122 is freely rotated to shoot then internal parts of the oral cavity, and the support piece is suitable for users of various ages (such as adults or children) and is with greater universality.

The thickness of at least the portion of the main body 11 is greater than the thickness of the support piece 122. On the one hand, the main body 11 has a certain thickness to provide sufficient accommodating space for internal components of the oral camera 100, and at the same time, it further provides a certain support for the user to hold the main body 11 on the other hand. The thickness of the support piece 122 is less than the thickness of the main body 11, which is convenient for the user to bite the support piece 122, which effectively reduces the opening degree of the mouth, thereby improving the user experience.

In addition, it is noted that the main body 11 is block-shaped and the support piece 122 is rod-shaped, so that the surface of the main body 11 in contact with the hand of the user is the flat surface, and the main body 11 is prevented from rolling when being held by the user. Further, the user is able to hold the main body 11 stably, thereby improving the stability of the user holding the main body 11, and facilitating the shooting of the desired target image. Moreover, the main body 11 is block-shaped to provide sufficient accommodating space for the internal components of the oral camera 100. The support piece 122 is rod-shaped, which is convenient for being inserted into the oral cavity. The user does not need to open the mouth very wide to realize the shooting function of the oral camera 100.

In some embodiments, a length-to-height ratio of the main body is less than a length-to-height ratio of the support piece 122. The length-to-height ratio of the main body is less than the length-to-height ratio of the support piece 122, so the main body 11 is close to a rectangular or square block structure, so that the main body 11 has sufficient holding space and storage space, and the support piece 122 is a long strip structure, which is convenient for being inserted into the oral cavity. to realize the shooting function.

In some embodiments, the width of the support piece 122 is greater than the thickness of the support piece 122. The support piece 122 is the long strip structure, so that the support piece 122 is easily inserted into the oral cavity to realize the shooting function of the oral camera 100.

Furthermore, in some embodiments, at least one of the side surfaces of the support piece 122 in the thickness direction is a flat surface. When the user bites on the side surfaces of the support piece 122 in the thickness direction and applies pressure, the at least one flat surface of the support piece evenly disperses the pressure along the thickness direction to prevent the at least one camera 121 from lateral displacement. In addition, when the side surfaces of the support piece 122 in the thickness direction abut against one of the dental arches for positioning (i.e. a positioning dental arch), the support piece 122 smoothly abut against the positioning dental arch to ensure the shooting angle and shooting quality of the at least one camera 121.

In some embodiments, the support piece 122 and the main body 11 are smoothly connected on the one side where the at least one camera 121 is disposed (as shown in FIG. 20), or a height difference between the connecting surfaces of the support piece 122 and the one side of the main body 11 where the at least one camera 121 is disposed is not greater than 5 mm. In this way, the connection position between the support piece 122 and the main body 11 is smooth without an abrupt connection structure. When the user inserts the support piece 122 into the oral cavity, the smooth connection of the support piece and the main body 11 reduces the stimulation and discomfort to the oral cavity and improves the user experience. Moreover, the smooth connection of the support piece and the main body 11 makes the oral camera 100 beautiful as a whole, meeting the aesthetic needs of the user.

For instance, the height difference between the connecting surface of the support piece 122 and the one side of the main body 11 where the at least one camera 121 is disposed may include but is not limited to 0, 1 mm, 2 mm, 3 mm, 4 mm, 5 mm, etc., which is not limited thereto. If the height difference between the connecting surface of the support piece 122 and the one side of the main body 11 where the at least one camera 121 is greater than 5 mm, the abrupt structure may be presented at the connection position between the support piece 122 and the main body 11, which not only affects the appearance and causes discomfort to the user when the support piece 122 is inserted into the mouth, but also reduces the user experience.

It is understood that the support piece 122 includes the two first sub-side surfaces 122e1, and the first sub-side surfaces 122e1t are opposite to each other in the thickness direction of the support piece. One of the first sub-side surfaces 122e1 of the support piece is the side surface of the support piece 122 where the at least one camera 121 is disposed. The connecting surface of the main body 11 connected to the side surface, where the at least one camera 121 is disposed, of the support piece is the first side surface 112a or the second side surface 112b of the main body 11. The one of the first sub-side surfaces 122e1 is substantially flush with the first side surface 112a or the second side surface 112b of the main body (as shown in FIG. 2), so the connection position of the support piece and the main body 11 is smoothly transitioned without the abrupt connection structure. When the user inserts the support piece 122 into the oral cavity, the connection position of the support piece and the main body 11 does not cause irritation and discomfort to the oral cavity, which improves the user experience. Moreover, the connection position of the support piece and the main body 11 is smoothly transitioned, which marks the appearance of the oral camera 100 beautiful as a whole to meet the aesthetic needs of the user.

In some embodiments, in the width direction of the support piece 122, a width of a position of the support piece 122 where the at least one camera 121 is disposed is greater than a width of other positions of the support piece 122. Alternatively, a recessed portion 1223 is defined on one side of the support piece 122 in the width direction. In this way, when the user shoots at least one of the inner surfaces of the upper teeth and the inner surfaces of the lower teeth, the user only needs to bite the two sides of the support piece 122 in the width direction except the at least one camera 121. The support piece is narrow in the width direction, which reduces the opening degree of the mouth of the user, thereby improving the user experience.

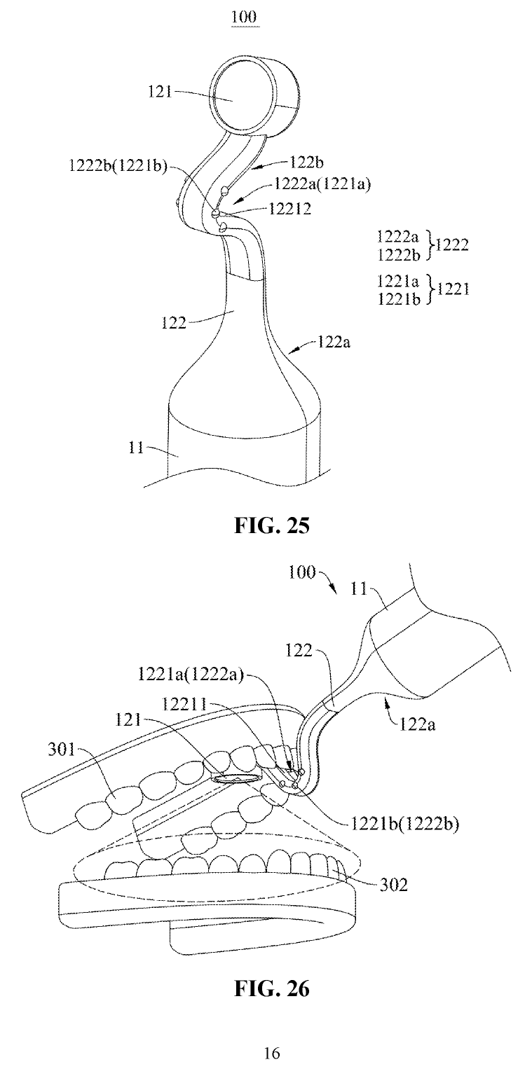

In some embodiments, as shown in FIG. 25, the support piece 122 includes at least one limiting portion 1221 disposed between the at least one camera 121 and the main body 11. The at least one limiting portion 1221 is configured to abut against the positioning dental arch to position the at least one camera 121 relative to the positioning dental arch. Specifically, the at least one limiting portion 1221 includes one or more limiting portions, which are not limited in the present disclosure. The at least one limiting portion 1221 limits the position and movement range of the support piece 122 in the oral cavity by abutting against the positioning dental arch (such as teeth, lips, etc.), ensuring that the at least one camera 121 on the support piece 122 maintains a relatively stable and accurate relative position with the teeth.

In the second form, the at least one limiting portion 1221 abuts against the positioning dental arch, thereby positioning the support piece 122 relative to the positioning dental arch, thereby positioning the at least one camera 121 relative to the positioning dental arch. Therefore, it is ensured that the at least one camera 121 is aligned with the occlusal surfaces and the inner surfaces of the teeth. Further, the stability and accuracy of shooting is improved, and image blur or shooting angle deviation caused by shaking or improper position of the support piece 122 is avoided. In addition, the at least one limiting portion 1221 provides guidance for the user, so the user is able to quickly find a better shooting position, and the at least one camera 121 obtains a better shooting angle. Finally, the panoramic image of the target dental arch in a single shot.

Optionally, a positional relationship between the at least one camera 121 and the positioning dental arch includes at least one of the depth of the at least one camera 121 inserted into the oral cavity, a distance from the at least one camera 121 to the positioning dental arch, and a rotation angle of the at least one camera 121 in the oral cavity.

When the at least one limiting portion 1221 is configured to limit the depth of the at least one camera 121 inserted into the oral cavity, the at least one limiting portion 1221 prevents the support piece 122 from inserting excessively into the oral cavity, reduces interference and potential damage to the soft tissue in the oral cavity, and improves the comfort and safety of the oral camera 100. In addition, by limiting the depth of the at least one camera 121 inserted into the oral cavity, the user is allowed to find the shooting position quickly, which further improves the ease of use, shortens the time required for shooting, and shortens the time required for adjusting the shooting position.

When the at least one limiting portion 1221 is configured to limit the distance from the at least one camera 121 to the positioning dental arch, the at least one camera 121 maintains a threshold distance required for shooting, so that it is easier to capture the panoramic image of the target dental arch, or when the panoramic image of the target dental arch is captured, the opening degree of the mouth is not too large.

When the at least one limiting portion 1221 is configured to limit the rotation angle of the at least one camera 121 in the oral cavity, when shooting, the optical axis of the at least one camera 121 is as perpendicular to the occlusal surfaces of the teeth as possible, or the angle of the optical axis of the at least one camera 121 and the occlusal surfaces of the teeth is maintained between 60° and 90°, so as to avoid the pits and the fissures of the occlusal surfaces being and unable to be fully imaged blocked due to excessive shooting angles. In addition, the optical axis of the at least one camera 121 is able to be fine-tuned to deviate toward the inner surfaces of the teeth so as to capture the corresponding inner surfaces of the teeth synchronously when shooting the occlusal surfaces of the teeth.

Optionally, the at least one limiting portion 1221 includes at least one of a groove, a protrusion and a bending structure. It should be noted that when the at least one limiting portion 1221 includes the groove, the groove is formed by removing part of the material of the support piece 122, or by the second peripheral side surface 122e of the support piece 122 being recessed inward (as shown in FIG. 15), or the groove is formed by the support piece 122 being bent and arched from an imaging side of the at least one camera 121 to an object side of the at least one camera 121 (as shown in FIG. 7-FIG. 8).

It should be noted that when the at least one limiting portion 1221 includes the protrusion, the at least one limiting portion 1221 may include one protrusion or protrusions, and the peripheral side surface of the one or more protrusions are pressed against the inner surfaces or the outer surfaces of the teeth for limiting or positioning, thereby limiting the depth of the at least one camera 121 inserted into the oral cavity.

In some embodiments, the at least one limiting portion includes the limiting portions 1221, such as two limiting portions 1221, three limiting portions 1221, four limiting portions 1221, five limiting portions 1221, six limiting portions 1221, seven limiting portions 1221 or eight limiting portions 1221, etc. The limiting portions 1221 are disposed at intervals along the length direction of the support piece 122.

The limiting portions 1221 adapt to individual differences of oral cavities of different users, especially adapt to the size of the dental arches and the arrangement of the teeth. Due to the differences in the size and shape of the dental arches of different users, such as the different tooth arrangement and oral depth of adults and children, the limiting portions 1221 provide a variety of limiting options, so that the support piece 122 is positioned by selecting one of the limiting portions 1221 according to an oral structure of the user, and limit the depths of the at least one camera 121 inserted into the oral cavity according to the oral structure of the user. That is, the at least one camera 121 is adjusted to the best position according to the size of the dental arches to capture the image of the inner surfaces of the dental arches. For example, when shooting the occlusal surfaces close to the posterior jaw side, one of the limiting portions 1221 that is relatively closer to the main body 11 is selected to ensure that the support piece 122 has enough length to insert into the oral cavity, thereby ensuring that the at least one camera 121 is inserted deep enough.

Exemplarily, the limiting portions 1221 include grooves disposed at intervals or protrusions disposed at intervals, so that a butted tooth is placed in a suitable groove or between two adjacent protrusions according to the oral structure of the user, so as to position the at least one camera 121 and limit the depths of the at least one camera 121 inserted into the oral cavity. Namely, the at least one camera 121 is adjusted to the optimal position according to the size of the dental arches of different users to capture the image of the inner surfaces of the dental arches.

When the limiting portions 1221 include the grooves, at least some of the grooves have different depths, and the depth of the grooves closer to the at least one camera 121 is smaller. Thus, the limiting portions 1221 are able to adapt to users (such as adults and children) with different sizes of the dental arch. A shallower groove thereof is close to the at least one camera 121 and is suitable for shooting the front teeth, because the front teeth (such as incisors and canines) are commonly small and shallow, and the shallower groove ensures that the at least one camera 121 maintains an appropriate distance from the front teeth, avoiding the distance between the at least one camera 121 and the teeth being too far caused by a deeper groove. Therefore, the shooting effect is not affected.

The deeper groove is suitable for shooting back teeth, such as molars. The back teeth are commonly larger and deeper, and the deeper groove is configured to accommodate one of the back teeth, while limiting the depth of the support piece 122 to ensure that the at least one camera 121 is aligned with the occlusal surfaces and the inner surfaces of the teeth.

The user may select an appropriate groove from the grooves for positioning according to the position of the teeth to be shot, thereby controlling the relative position and angle between the at least one camera 121 and the teeth. For example, when shooting the teeth of children, the shallower groove is selected to adapt to the shallower oral structure; when shooting the teeth of adults, the deeper groove is selected to adapt to the deeper oral structure.

When the limiting portions 1221 include the protrusions, at least two of the protrusions have differe4nt heights relative to the support piece 122, and one of the teeth is sandwiched in two adjacent protrusions for positioning the support piece. The closer the protrusions are to the at least one camera 121, the smaller the height of the protrusions. Therefore, the support piece is allowed to adapt to users with different sizes of dental arches, such as adults and children.

Adjacent lower protrusions thereof are close to the at least one camera 121 and are suitable for shooting the front teeth, because the front teeth (such as incisors and canines) are commonly small and shallow, and the adjacent lower protrusions ensure that the at least one camera 121 maintains an appropriate distance from the front teeth, avoiding the distance between the at least one camera 121 and the teeth being too far caused by adjacent higher protrusions. Therefore, the shooting effect is not affected.

The user may select appropriate adjacent protrusions from the protrusions for positioning according to the position of the teeth to be shot, thereby controlling the relative position and angle between the at least one camera 121 and the teeth. For example, when shooting the teeth of children, the adjacent lower protrusions are selected to adapt to the shallower oral structure. When shooting the teeth of the adults, the adjacent higher protrusions are selected to adapt to the deeper oral structure.

In some embodiments, as shown in FIG. 20, the support piece 122 includes a second peripheral side surface 122e perpendicular to the length direction thereof. The at least one limiting portion 1221 and the at least one camera 121 are disposed at intervals on the second peripheral side surface 122e. The at least one limiting portion 1221 and the at least one camera 121 are located on two opposite sides or two adjacent sides of the support piece 122. The at least one limiting portion 1221 is disposed between the at least one camera 121 and the main body 11.

Specifically, the at least one limiting portion 1221 and the at least one camera 121 are disposed at intervals, which means that there is a certain distance between the at least one limiting portion 1221 and the at least one camera 121. The at least one limiting portion 1221 and the at least one camera 121 are disposed on opposite sides of the support piece 122. In the second form, the at least one limiting portion 1221 abuts against the canines and other parts of the teeth. After the at least one limiting portion 1221 abuts against the teeth, the at least one camera 121 is aligned with the maxillary teeth and/or mandibular teeth from the direction opposite to the at least one limiting portion 1221, thereby obtaining the image of the occlusal surfaces and the inner surface of the maxillary teeth and/or mandibular teeth.

When the at least one limiting portion 1221 and the at least one camera 121 are disposed on two adjacent sides of the support piece. In the second form, the at least one limiting portion 1221 abuts against the canines and other parts of the teeth. After the at least one limiting portion 1221 abuts against the teeth, the at least one camera 121 faces two sides of the mouth, thereby capturing images of the inner surfaces of the teeth on the left and right sides of the mouth. The left side and the right side of the mouth are defined relative to the human body, such as the left and right ears on two sides of the human body are defined relative to the human body.

When the at least one limiting portion 1221 and the at least one camera 121 are located on the same side, in the second form, the at least one limiting portion 1221 abuts against the canines or other parts of the teeth, limiting the position and movement range of the support piece 122 in the mouth, and preventing the support piece 122 from excessively inserting into the mouth.

In some embodiments, in the length direction of the support piece 122, a distance between the at least one limiting portion 1221 and the at least one camera 121 is not less than 5 mm and not greater than 20 mm.

If the distance between the at least one limiting portion 1221 and the at least one camera 121 is less than 5 mm, when the at least one limiting portion 1221 clamps on the positioning dental arch, the at least one camera 121 may not be able to fully insert into the oral cavity, resulting in the at least one camera 121 being unable to obtain an image of the teeth deep in the oral cavity, and the shooting range is limited. If the distance between the at least one limiting portion 1221 and the at least one camera 121 is greater than 20 mm, when the at least one limiting portion 1221 clamps on the positioning dental arch, the support piece 122 may be excessively inserted into the oral cavity, reducing interference and potential damage to the soft tissues in the oral cavity (such as the gums and the tongue).

Therefore, the distance between the at least one limiting portion 1221 and the at least one camera 121 is not less than 5 mm and not greater than 20 mm. On the one hand, when the at least one limiting portion 1221 clamps on the positioning dental arch, it is ensured that the at least one camera 121 is inserted into the oral cavity to obtain a more comprehensive and in-depth image of the interior of the oral cavity, such as the image of the inner surfaces and the occlusal surfaces of the molars close to the throat. On the other hand, the support piece 122 is prevented from excessively inserting into the oral cavity, thereby reducing interference and potential damage to the soft tissues in the oral cavity (such as the gums and the tongue).

In some embodiments, the at least one limiting portion 1221 includes at least one of a first limiting portion 1221a and second limiting portions 1221b. The first limiting portion 1221a and the at least one camera 121 are disposed on two opposite sides of the support piece 122. When the second limiting portions and the at least one camera 121 are disposed on two adjacent sides of the support piece 122, the first limiting portion 1221a and the second limiting portions 1221b are configured to limit the depth of the support piece 122 inserting into the oral cavity, thereby preventing the support piece 122 from inserting excessively into the oral cavity, and reducing interference and potential damage to the soft tissues in the oral cavity (such as the gums and the tongue). In addition, by limiting the depth of the at least one camera 121 inserted into the oral cavity, the user is guided to find the shooting position faster, thereby further improving ease of use, shortening the time required for shooting, and shortening the time required for adjusting the position of the at least one camera.

In some embodiments, when the at least one limiting portion 1221 includes the first limiting portion 1221a, the first limiting portion 1221a is further configured to increase the distance between the at least one camera 121 and the dental arches to be shot, so that the at least one camera 121 maintains the threshold distance required for shooting, and it is easier to capture the panoramic image, or when the panoramic image is captured, the opening degree of the mouth is not too large.

In some embodiments, the first limiting portion 1221a includes the groove, which is formed by removing part of the material of the support piece 122, or by the first sub-side surface 122e1 of the support piece 122 being recessed inwardly (as shown in FIG. 20), or the groove is formed by bending the support piece 122 toward the light incident side of the at least one camera 121 (as shown in FIGS. 22-24). Specifically, the support piece 122 abuts against the first dental arch 301 through the first limiting portion 1221a. Therefore, the distance between the at least one camera 121 and the target dental arch (the second dental arch 302, such as the occlusal surfaces of the second dental arch 302) increases via the first limiting portion. Alternatively, the support piece 122 abuts against the second dental arch 302 through the first limiting portion 1221a, and the distance between the at least one camera 121 and the target dental arch (the first dental arch 301, such as the occlusal surfaces of the first dental arch 302) increases via the first limiting portion 1221a.

When the support piece 122 is placed against the first dental arch 301 through the groove, the at least one camera 121 is placed below or above the highest point of the second dental arch 302 in the mouth. Therefore, the at least one camera 121 is positioned in the oral cavity relative to the shot teeth to increase the distance between the second dental arch 302 and the at least one camera 121, which enables the at least one camera 121 to obtain the panoramic image of the second dental arch 302 in a single shot. Further, in this case, the user does not need to open the mouth too wide.

In some embodiments, as shown in FIG. 23, when the first limiting portion 1221a is the groove, in the direction from the object side of the at least one camera 121 to the imaging side of the at least one camera 121 (such as the z1 direction in FIG. 23), the bottom surface of the groove is lower than the light incident surface of the at least one camera 121. That is, in the direction from the object side of the at least one camera 121 to the imaging side of the at least one camera 121, the bottom surface of the groove is observed first, and then the light incident surface of the at least one camera 121 is observed. Therefore, when the bottom surface of the groove abuts against the cusp of one of the teeth of the lower dental arch, the at least one camera 121 is placed below the highest point of the lower dental arch in the mouth, thereby increasing the distance between the at least one camera 121 and the upper dental arch, achieving a larger range or wider shooting angle of the upper dental arch, and obtaining a more complete or wider image of the upper dental arch. Alternatively, when the bottom surface of the groove abuts against the cusp of one of the teeth of the upper dental arch, the at least one camera 121 is placed above the highest point of the upper dental arch in the mouth, thereby increasing the distance between the at least one camera 121 and the lower dental arch, achieving a larger range or wider shooting angle of the lower dental arch, and obtaining a more complete or wider image of the upper dental arch.

In some embodiments, a depth of the first limit portion 1221a (i.e., the groove) is not less than 3 mm and not greater than 15 mm. For example, the depth of the groove is 3 mm, 4 mm, 4.5 mm, 5 mm, 5.5 mm, 6 mm, 6.5 mm, 7 mm, 8 mm, 8.5 mm, 9 mm, 10 mm, 10.5 mm, 11 mm, 12 mm, 12.5 mm, 13 mm, 14 mm, 14.5 mm or 15 mm, etc.

If the depth of the groove is less than 3 mm, a height of the at least one camera 121 relative to the cusp of the one of the teeth is not enough, and the at least one camera 121 is unable to shoot a larger range or a wider angle of the one of the dental arch. If the depth of the groove is greater than 15 mm, a risk of the support piece 122 touching the gums may increase, affecting the shooting effect and user experience.

Therefore, the depth of the groove is between 3-15 mm, which reduces the risk of the support piece 122 colliding with the gums while maintaining the distance between the at least one camera 121 and the positioning dental arch at an optimal shooting distance, thereby achieving a larger range or wider shooting angle of the one of the dental arches and obtaining a more complete or wider image of the target dental arch.

In some embodiments, the first limiting portion 1221a includes a bending structure, and a bending direction of the bending structure faces the side away from the at least one camera 121, so as to increase the distance between the target dental arch and the at least one camera 121, so that the at least one camera 121 is able to obtain the panoramic image of the target dental arch in a single shot. Such a configuration further significantly reduces the opening degree of the mouth that the user needs to open when shooting.

As shown in FIG. 24, the bending structure enables the at least one camera 121 to be inclined on the support piece 122, and the angle between the insertion end 122b of the support piece 122 and the length direction of the support piece 122 is 20°-40°. Such a configuration increases the distance between the at least one camera 121 and the teeth to be shot, so that it is easier to obtain the panoramic image of the target dental arch.

In some embodiments, when the at least one limiting portion 1221 includes the first limiting portion 1221a, the support piece 122 is against the first dental arch 301 through the first limiting portion 1221a to shoot the second dental arch 302, and the at least one limiting portion 1221 is configured to limit the rotation angle of the at least one camera 121. Specifically, the first limiting portion 1221a (i.e, the groove) of the support piece 122 abuts against the inner surface or the outer surfaces of the teeth of the first dental arch 301 (or the second dental arch 302), thereby limiting the rotation angle of the at least one camera 121. Therefore, when shooting, the optical axis of the at least one camera 121 should be as perpendicular to the occlusal surfaces of the teeth of the second dental arch 302 (or the first dental arch 301) as possible, or the angle between the at least one camera and the occlusal surfaces of the teeth is maintained between 60° and 90°, so as to avoid the pits and the fissures of the occlusal surfaces of the teeth being blocked due to excessive shooting angles. Therefore, the panoramic image of the occlusal surfaces of the teeth is obtained. In addition, the optical axis of the at least one camera 121 is fine-tuned to deviate to the inner surfaces of the teeth, so as to capture the corresponding inner surfaces of the teeth synchronously when shooting the occlusal surfaces of the teeth.

In the depth direction of the oral cavity, an inclination angle of the at least one camera is adjusted according to the angle between the optical axis of the at least one camera 121 and the occlusal surfaces of the teeth. In the lateral direction of the oral cavity (left and right direction), the surface of the support piece 122 where the first limiting portion 1221a is located (that is, the surface of the support piece 122 in contact with the teeth, excluding the first limiting portion 1221a) is smooth or flat, so that the surface of the support piece 122 in contact with the teeth is stably placed without lateral deviation. That is, the distance between the at least one camera 121 and the occlusal surfaces of the left teeth is basically equal to the distance between the at least one camera 121 and the occlusal surfaces of the right teeth, which improves the stability of the support piece 122 during operation.

In some embodiments, when the oral imaging device 1000 abuts against the first dental arch 301 or the second dental arch 302 through the first limiting portion 1221a, the rotation angle of the optical axis of the at least one camera 121 is variable within a range of no more than 60°. Namely, an adjustable range of the shooting angle of the at least one camera 121 is not greater than 60°. By such a configuration, it is ensured that the optical axis of the at least one camera 121 is as perpendicular to the occlusal surfaces of the teeth as possible when shooting. Alternatively, when the angle of the optical axis of the at least one camera 121 and the occlusal surfaces of the teeth is maintained between 60° and 90°, the optical axis of the at least one camera 121 is slightly adjusted to incline toward the inner surfaces of the tooth, so that the occlusal surfaces and the corresponding inner surfaces of the teeth are shot at the same time.

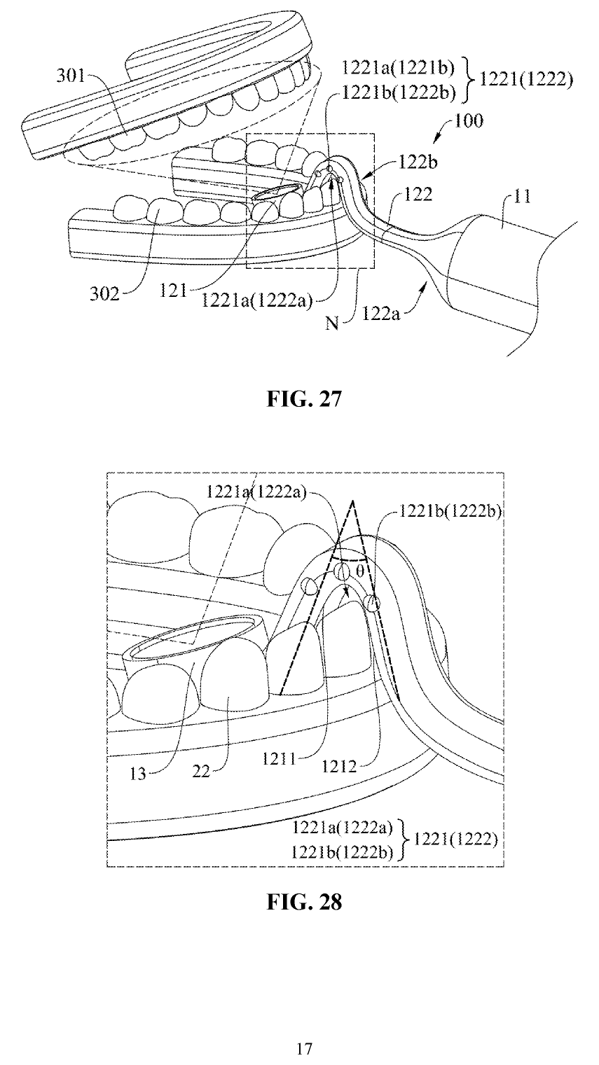

In some embodiments, as shown in FIGS. 25-28, when the at least one limiting portion 1221 includes the first limiting portion 1221a, and the first limiting portion 1221a is the groove. The groove includes a first abutting surface 12211 and a second abutting surface 12212 opposite to the first abutting surface 12211 in the length direction of the support piece 122. In the depth direction of the groove, the distance between the first abutting surface 12211 and the second abutting surface 12212 in the length direction of the support piece 122 decreases. In this way, when the oral camera 100 abuts against the first dental arch 301 or the second dental arch 302 through the first limiting portion 1221a, the probability of the first limiting portion 1221a contacting the gums is reduced.

In some embodiments, when the groove accommodates one of the teeth, the first abutting surface 12211 of the groove abuts against an inner surface of the one of the teeth, and/or the second abutting surface 12212 of the groove abuts against an outer surface of the one of the teeth to limit the range of the angle between the optical axis of the at least one camera 121 and the occlusal surfaces of the teeth within a preset angle. For example, the angle between the optical axis of the at least one camera 121 and the occlusal surfaces of the teeth is 60°-90°, thereby ensuring that when shooting, the shooting angle is not too large, avoiding a problem that the pits and the fissures of the occlusal surfaces of the teeth being blocked by the cusp of the occlusal surfaces of the teeth, and obtaining the panoramic image of the occlusal surfaces of the teeth.

If the angle formed by the optical axis of the at least one camera 121 and the occlusal surfaces of the teeth is less than 60°, the shooting angle is too low, and the pits and the fissures of the occlusal surfaces of the teeth may be blocked by contact surfaces of the upper teeth and the lower teeth, such that the characteristics of the occlusal surfaces of the teeth are not fully displayed.

The angle between the optical axis of the at least one camera 121 and the occlusal surfaces of the teeth is not less than 60° and not greater than 90°, which effectively avoids excessive inclined angle of the at least one camera 121, so as to avoid the pits and the fissures of the occlusal surfaces of the teeth being blocked due to excessive shooting angles. Therefore, the panoramic image of the occlusal surfaces of the teeth is obtained.

In some embodiments, an angle between the first abutting surface 12211 of the groove and the second abutting surface 12212 of the groove is not less than 30°.

In some embodiments, the angle between the first abutting surface 12211 of the groove and the second abutting surface 12212 of the groove is not greater than 60°.

For example, the angle between the first abutting surface 12211 of the groove and the second abutting surface 12212 of the groove is 30°, 35°, 40°, 45°, 50°, 55° or 60°.

When the first limiting portion 1221a fits the teeth, the first abutting surface 12211 abuts against the inner surface of the one of the teeth, or the second abutting surface 12212 of the groove abut against the outer surface of the one of the teeth to limit the angle of the support piece 122,. Namely, the angle of the at least one camera 121 is limited. The angle between the first abutting surface 12211 of the groove and the second abutting surface 12212 of the groove is controlled between 30° and 60°, which ensures that the optical axis of the at least one camera 121 is as perpendicular to the occlusal surfaces of the target dental arch as possible when shooting. Alternatively, the angle of the optical axis of the at least one camera 121 and the occlusal surfaces of the teeth is maintained between 60° and 90°, so as to avoid the problem that the pits and the fissures of the occlusal surfaces of the teeth being blocked due to excessive shooting angles. Therefore, the panoramic image of the occlusal surfaces of the teeth is obtained.

In some embodiments, when the at least one limiting portion 1221 includes the groove, a wall surface of the groove in the length direction of the support piece 122 is arc-shaped to adapt to the shape of the teeth, so as to facilitate the support piece 122 to stably clamp the one of the teeth, stabilize the at least one camera 121, and prevent the at least one camera 121 from shaking, thereby improving the shooting quality of the at least one camera 121.

In some embodiments, the at least one limiting portion 1221 includes the protrusions. Peripheral side surfaces of the protrusions in the length direction of the support piece 122 are arc-shaped so that a space defined between each two adjacent protrusions is adapted to the shape of the teeth (such as the incisors), thereby facilitating the support piece 122 to stably clamp the one of the teeth, stabilize the at least one camera 121, and prevent the at least one camera 121 from shaking, thereby improving the shooting quality of the at least one camera 121.

In some embodiments, the at least one limiting portion 1221 includes the protrusions, and a connection position between a peripheral side surface of each of the protrusions and an end surface thereof is chamferred, or one end of each of the protrusions away from the support piece 122 is a dome. Therefore, when the support piece 122 abuts between the first dental arch 301 and the second dental arch 302 through the at least one limiting portion 1221, it is convenient to rotate the support piece 122 to adjust the shooting angle of the at least one camera 121.

In some embodiments, when the at least one limiting portion 1221 includes the groove, a size of the groove in the length direction of the support piece 122 is matched with the thickness of the teeth, and/or a depth of the groove is matched with the height of the teeth.

When the size of the groove in the length direction of the support piece 122 is matched with the thickness of the teeth, the support piece 122 is well limited by the at least one limiting portion 1221, so that the support piece 122 is relatively stable against the positioning dental arch, avoiding the shaking of the at least one camera 121, thereby improving the shooting quality of the at least one camera 121.

When the depth of the groove is matched with the height of the teeth, the probability of the support piece 122 contacting the gums is reduced.

Optionally, in the length direction of the support piece 122, the size of the groove is not less than 3 mm; and/or the depth of the groove is not greater than 8 mm.

By making the size of the groove in the length direction of the support piece 122 not less than 3 mm, the support piece 122 is well limited by the at least one limiting portion 1221, so that the support piece 122 is relatively stably against the positioning dental arch, thereby avoiding the shaking of the at least one camera 121 and improving the shooting quality of the at least one camera 121.

By making the depth of the groove not greater than 8 mm, the probability of the support piece 122 contacting the gums is reduced. Furthermore, the depth of the groove does not exceed 3.5 mm, which prevents the support piece 122 from contacting the gums while allowing the support piece 122 to be relatively stably against the positioning dental arch through the at least one limiting portion 1221. Thus, the support piece is movable along a predetermined trajectory as a fulcrum of a seesaw.

In some embodiments, when the at least one limiting portion 1221 includes the protrusions, the distance between each two adjacent protrusions is matched with the thickness of the teeth, and/or the height of each of the protrusions relative to the support piece 122 is matched with the height of the teeth.

When the distance between each two adjacent protrusions is matched with the thickness of the teeth, the support piece 122 is well limited by the space defined between each two adjacent protrusions, so that the support piece 122 is relatively stably against the positioning dental arch, thereby avoiding the shaking of the at least one camera 121 and improving the shooting quality of the at least one camera 121.

When the height of each of the protrusions relative to the support piece 122 is matched with the height of the teeth, the probability of the support piece 122 contacting the gums is reduced.

Optionally, in the length direction of the support piece 122, the distance between each two adjacent protrusions is not less than 3 mm, and/or the height of each of the protrusions relative to the support piece 122 is not greater than 8 mm.

By making the distance between each two adjacent protrusions in the length direction of the support piece 122 not less than 3 mm, the support piece 122 is well limited by the space defined between each two adjacent protrusions, so that the support piece 122 is relatively stably against the positioning dental arch, thereby avoiding the shaking of the at least one camera 121 and improving the shooting quality of the at least one camera 121.

By making the height of each of the protrusions relative to the support piece 122 l not greater than 8 mm, the probability of the protrusion contacting the gums is reduced.

Considering that, under normal circumstances, when the support piece 122 is inserted into the mouth of the user, the surface of the support piece 122 along the width direction thereof or the thickness direction thereof is commonly being placed against the positioning dental arch.

Therefore, in some embodiments, the surface of the support piece 122 configured to contact the positioning dental arch remains straight, smooth, or flat in the width direction of the support piece 122. On the one hand, such a design enables the support piece 122 to well fit the positioning dental arch when the support piece is inserted into the mouth of the user, thereby improving the stability and accuracy of shooting. Further, such a design reduces the foreign body sensation of the user to the oral camera 100 to a certain extent, making shooting easier. On the other hand, when the user bites and applies pressure to the support piece 122 (the at least one limiting portion 1221), the straight, smooth or flat surface of the support piece evenly disperses the pressure along the width direction of the support piece 122, thereby effectively preventing the at least one camera 121 from lateral displacement on the positioning dental arch, so that the distance from the at least one camera 121 to the left and right areas of the positioning dental arch is relatively consistent.

In some embodiments, the surface of the support piece 122 is configured to contact the positioning dental arch remains straight, smooth, or flat in the thickness direction of the support piece 122. On the one hand, such a design enables the support piece 122 to well fit the positioning dental arch when the support piece is inserted into the mouth of the user, thereby improving the stability and accuracy of shooting. On the other hand, when the user bites and applies pressure, the straight, smooth or flat surface of the support piece evenly disperses the pressure along the thickness direction of the support piece 122, thereby preventing the at least one camera 121 from lateral displacement.

It is understood that the term "smooth" means that the surface has no significant protrusions or depressions, presenting a relatively smooth state. The term "straight" means that the surface is linearly extended in a certain direction (such as the width direction of the support piece 122) without bending or twisting.

In some embodiments, as shown in FIGS. 25-28, the support piece 122 includes at least one abutting portion 1222 configured to abut against the positioning dental arch. When the support piece 122 abuts against the first dental arch 301, the at least one camera 121 is kept stable by the at least one abutting portion 1222, and the at least one camera 121 is configured to capture at least a first image, and the first image includes the image of at least a first part of the second dental arch 302.

In this way, the at least one abutting portion 1222 abuts against the positioning dental arch to limit the position of the at least one camera 121 in the oral cavity and/or stabilize the at least one camera 121, so as to provide a good position and angle for dental arch shooting, so that the at least one camera 121 has a good shooting angle, thereby achieving a large shooting range or the wide shooting angle of the target dental arch. Finally, the complete or wide image of the target dental arch is obtained, and the entire target dental arch is captured in a single image without the reflector. The operation is relatively simple, and the image of the occlusal surfaces with a good viewing angle is obtained, and the shooting is completed by a single person, and the user is able to detect the oral cavity by himself/herself. That is, the user may act as the dentist and the patient at the same time.

In some embodiments, the at least one abutting portion 1222 includes a first abutting portion 1222a, and the first abutting portion 1222a and the at least one camera 121 are disposed on opposite sides of the support piece 122. The first image includes the image of the occlusal surfaces of the upper teeth or the occlusal surfaces of the lower teeth.

For example, as shown in FIGS. 25-28, the support piece 122 is placed against the incisor of the lower dental arch by the first abutting portion 1222a to capture the image of the occlusal surfaces of the upper dental arch. Alternatively, the first abutting portion 1222a is placed against the incisor of the upper dental arch to capture the image of the occlusal surfaces of the lower dental arch.

The at least one camera 121 is stabilized by the at least one abutting portion 1222 to obtain the image of the occlusal surfaces of the upper dental arch that is more complete and wider or the image of the occlusal surfaces of the lower dental arch that is more complete and wider. Therefore, the oral imaging device 1000 in the present disclosure is allowed to capture the image of the entire upper dental arch and the image of the entire lower dental arch.

In some embodiments, the first image includes the panoramic image of the occlusal surfaces of the upper teeth or the panoramic image of the occlusal surfaces of the lower teeth. The panoramic image of the occlusal surfaces of the upper teeth and the panoramic image of the occlusal surfaces of the lower teeth play a key supporting role in improving the detection efficiency and meeting the requirements for dentition arrangement detection. The panoramic image of the occlusal surfaces of the upper teeth and the panoramic image of the occlusal surfaces of the lower teeth are images that can only be captured by a SLR camera, the mouth opener 200 and the reflector in various detection scenarios. The oral imaging device 1000 in the present disclosure is allowed to capture the image of the entire upper dental arch or the image of the entire lower dental arch in a single shot, which reduces the number of shots, saves shooting time, improves patient comfort, and expands the detection of diseases related to dentition arrangement that is detected by a conventional oral imaging device.

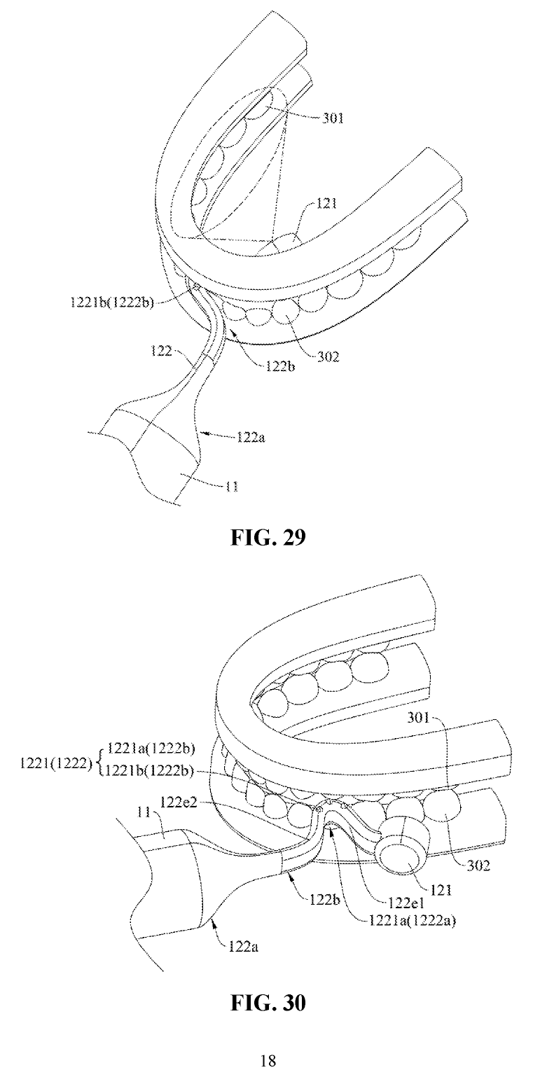

In some embodiments, as shown in FIGS. 29-30, the at least one abutting portion 1222 includes second abutting portions 1222b, and the second abutting portions 1222b and the at least one camera 121 are disposed on adjacent sides of the support piece 122. The support piece 122 abuts against at least one of the first dental arch 301 and the second dental arch 302 through the second abutting portions 1222b, and the support piece 122 is located between the first dental arch 301 and the second dental arch 302. The at least one camera 121 is configured to capture the image of the inner surfaces of the upper teeth and/or the inner surface of the lower teeth.

The at least one camera 121 is stabilized by the at least one abutting portion 1222 to obtain the image of the inner surfaces of the upper teeth with a better position and angle and/or the inner surface of the lower teeth, so as to obtain a more complete or wider image of the upper dental arch or lower dental arch. Therefore, the oral imaging device 1000 in the present disclosure is allowed to capture the image of the entire upper dental arch and the image of the entire lower dental arch.

It should be noted that, in some embodiments, the first abutting portion 1222a and the first limiting portion 1221a are the same structure, and the specific structure of the first abutting portion 1222a is detailed in the above description of the first limiting portion 1221a, which is not repeatedly described herein. In some embodiments, the second abutting portions 1222b and the second limiting portions 1221b are the same structures, and the specific structures of the second abutting portions 1222b are detailed in the above description of the second limiting portions 1221b, which are not repeatedly described herein.

In other words, in some embodiments, the first limiting portion 1221a and the first abutting portion 1222a are disposed selectively, or the second limiting portions 1221b and the second abutting portions 1222b are disposed selectively. For example, when the first limiting portion 1221a is disposed on the side of the support piece 122 opposite to the at least one camera 121, the first limiting portion 1221a realizes the function of the first abutting portions 1222a. That is, the first dental arch 301 abuts against the first limiting portion 1221a, so as to limit the position of the at least one camera 121 and maintain the stability of the at least one camera 121. For another example, when the second limiting portions 1221b are disposed on two opposite sides of the support piece 122 along the width direction, the second limiting portions 1221b realize the function of the second abutting portions 1222b. That is, the first dental arch 301 abuts against the second limiting portions 1221b, so as to limit the depth of the at least one camera 121 inserting into the oral cavity, thereby preventing the support piece 122 from inserting excessively into the oral cavity. Thus, the shooting position is quickly positioned.

Of course, in other embodiments, the first limiting portion 1221a and the first abutting portion 1222a are provided at the same time, and/or the second limiting portions 1221b and the second abutting portions 1222b are provided at the same time, which is not limited in the embodiments of the present disclosure.

As shown in FIG. 29, when the support piece 122 is laterally against one side of the positioning dental arch (e.g., the second dental arch 302) or is placed between the first dental arch 301 and the second dental arch 302, the second abutting portions 1222b stabilize the support piece 122, which enables the at least one camera 121 to face the inner surfaces of at least a portion of the upper and lower dental arches. When shooting the dental arches (as shown in FIG. 29 ), the user or the dentist inserts the at least one camera 121 into the mouth of the user, the support piece 122 is positioned through the second abutment portions 1222b. The second abutting portions 1222b limit the position of the at least one camera 121: so the at least one camera 121 either abuts against the second dental arch 302 or is roughly positioned between the two dental arches. Therefore, the at least one camera 121 is able to capture the image of at least a portion of the inner surfaces of the upper and lower dental arches.

The teeth of the user abut against the second abutting portions 1222b to form a fulcrum, and when the user or the dentist rotates or moves the main body 11, the at least one camera 121 rotates accordingly. The at least one camera rotates to shoot the inner surfaces of the upper and lower dental arches from different angles as needed. For example, to obtain a good viewing angle of the molars, the user or the dentist may rotate the at least one camera 121 to a position away from the molars to obtain an image of the inner surfaces of the molars.

In some embodiments, the second peripheral side surface 122e of the support piece 122 perpendicular to the length direction of the support piece includes sub-side surfaces opposite to each other. The second abutment portions 1222b are disposed on at least one of the sub-side surfaces of the support piece. Specifically, when the at least one camera 121 is disposed on the first sub-side surfaces 122e1 of the support piece 122 in the thickness direction, the second abutting portions 1222b are at least disposed on one of the second sub-side surfaces 122e2 of the support piece in the width direction. For instance, the second abutting portions 1222b are disposed on the two second sub-side surfaces 122e2 in the width direction.

In some embodiments, the sub-side surfaces are flat surfaces, or the bottom surfaces of the second abutting portions 1222b are flat surfaces. In this way, when the patient bites the support piece and applies pressure, the sub-side surfaces evenly disperse the pressure along the thickness direction of the support piece 122 to prevent the at least one camera 121 from lateral displacement.

In some embodiments, the first sub-side surfaces are parallel to each other, and the second sub-side surfaces are parallel to each other, or, when the second abutting portions 1222b are disposed on the two second sub-side surfaces 122e2 of the support piece 122 in the width direction, the bottom surfaces of the second abutting portions 1222b on the two second sub-side surfaces 122e2 are parallel. In this way, when the patient bites the support piece and applies pressure, the width of the support piece 122 at the biting position in the width direction is the same, which is conducive to the patient's bite normally and avoids deviation.

In some embodiments, the at least one abutting portion 1222 includes the first abutting portion 1222a and the second abutting portions 1222b. When the first abutting portion 1222a abuts against the first dental arch 301 to shoot the occlusal surfaces of the second dental arch 302, the at least one camera 121 is movable along a first path. When the second abutting portion 1222b abuts against at least one of the first dental arch 301 and the second dental arch 302 to shoot the inner surfaces of the upper teeth and/or the inner surfaces of the lower teeth, the at least one camera 121 is movable along a second path. A plane where the first path is located intersects with a plane where the second path is located.

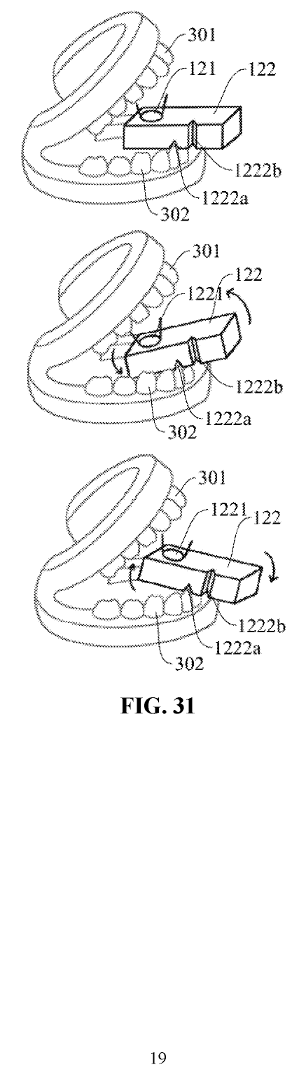

As shown in FIG. 31, the first path is a path along which the at least one camera 121 is movable when the support piece 122 is placed against the positioning dental arch, and the at least one camera 121 faces the target dental arch through the first abutting portion 1222a to capture the image of the occlusal surfaces of the target dental arch. At this time, the first abutting portion 1222a is configured as a fulcrum, allowing the user to push the main body 11 (such as a handle) up and down, so that the at least one camera 121 rotates in the vertical direction. Therefore, the user is allowed to quickly switch the viewing angle according to specific needs, ensuring that the at least one camera 121 is able to capture images from different angles, reducing blind spots or information omissions caused by a single viewing angle without complicated adjustment steps, and improving operational efficiency and convenience.

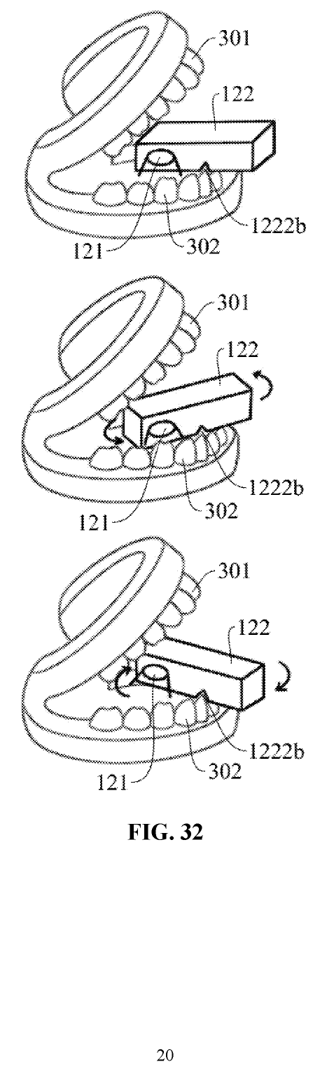

As shown in FIG. 32, the second path is a moving path of the at least one camera 121 when the support piece 122 is placed against the positioning dental arch and the at least one camera 121 is stabilized by the second abutting portions 1222b to capture the image of the inner surfaces of the teeth. At this time, the second abutting portions 1222b are configured as a fulcrum, allowing the user to push the main body 11 left and right, so that the at least one camera 121 rotates in the horizontal direction. Therefore, the user is allowed to quickly switch the viewing angle according to specific needs, ensuring that the at least one camera 121 is able to capture images from different angles, reducing blind spots or information omissions caused by a single viewing angle without complicated adjustment steps, and improving operational efficiency and convenience.

In some embodiments, the plane where the first path is located is perpendicular to the plane where the second path is located. The at least one camera 121 is movable and adjustable in two different directions, providing the user with operational freedom. For example, under the first path, the at least one camera 121 moves along a vertical or nearly vertical plane to capture the image of the inner surfaces of the teeth. Under the second path (located inside the mouth), the at least one camera 121 moves in the plane perpendicular to the first path. That is, the at least one camera 121 moves along a horizontal or nearly horizontal plane to capture the image of the occlusal surfaces of the teeth. The two paths do not interfere with each other, allowing users to quickly switch the viewing angles according to specific needs without the need for complex adjustment steps, thereby improving operational efficiency and convenience. The at least one camera 121 under the two paths obtains image data in different dimensions, thereby providing a more comprehensive and detailed view, ensuring that the at least one camera 121 is able to capture the images from different angles, and reducing the blind spots or the information omissions caused by a single viewing angle.

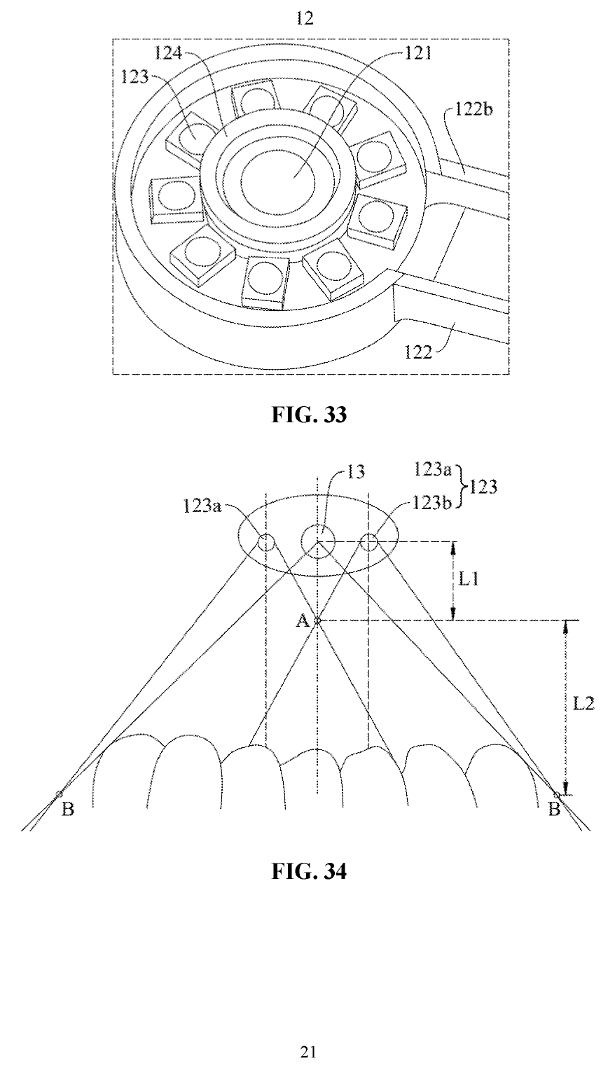

In some embodiments, as shown in FIG. 33, the camera module 12 further includes light sources surrounding the at least one camera, so that the light sources provide sufficient light to enhance a visualization effect of the pathological condition, thereby obtaining a clear image of the target oral region.

In some embodiments, the light sources are configured to generate white light, blue light, purple light, ultraviolet light or infrared light. A wavelength range of the light sources is selected from at least one of: 315-400nm (ultraviolet light), 380-750nm (visible light, the white light), 450-500nm (the blue light) or 750-1500nm (the infrared light). The light sources not only provide sufficient illumination for the at least one camera, but also emit light of a specific wavelength to assist detection. For example, the ultraviolet light is able to stimulate a fluorescence effect of dental plaque.

In some embodiments, the camera module 12 further includes at least one light shield 124 disposed around the at least one camera, and the light sources are disposed around the at least one light shield 124. In this way, the at least one light shield 124 is configured to limit the light path of the light source, thereby preventing the light sources from forming a light spot or a ghost image in the at least one camera, thereby improving the imaging quality of the at least one camera.

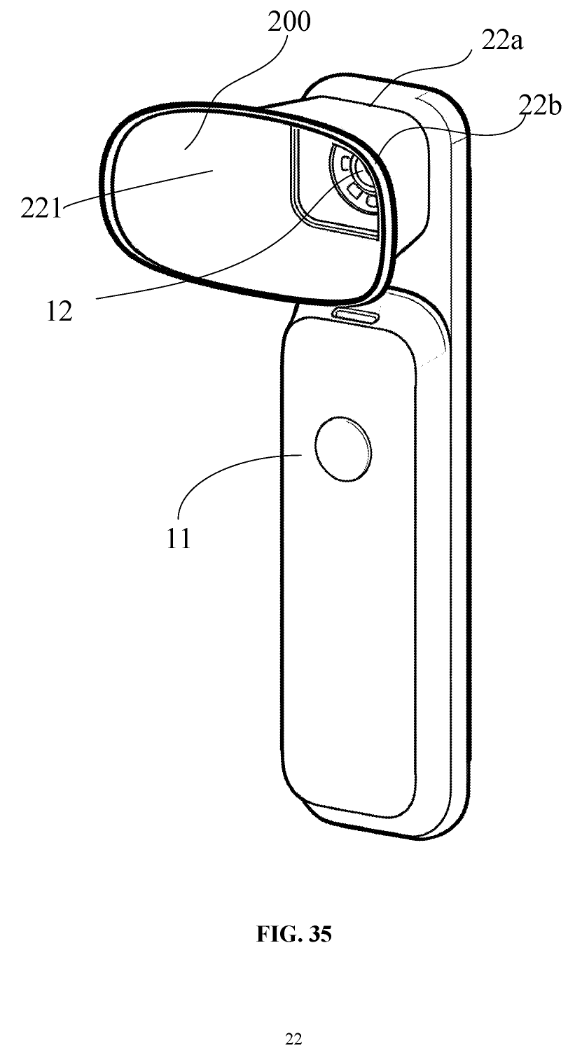

For ease of description, as shown in FIG. 34, one of two farthest light sources of the light sources is defined as a first light source 123a, and the other one thereof is defined as a second light source 123b. An intersection of a contour line of a light emitting range of the first light source 123a and a contour line of a light emitting range of the second light source 123b is defined as a first intersection point A. An intersection of the contour line of the light emitting range of any one of the first light source 123a and the second light source 123b and a contour line of the shooting range of the at least one camera 121 is defined as a second intersection point B;

A distance from the first intersection point A to a surface where the light sources are disposed is configured as L1. That is, a distance between an intersection of the light paths of the at least one camera 121 and each of the light sources is L1, a distance between the first intersection point A and the second intersection point B is configured as L2. That is, a distance between the light paths of the at least one camera 121 and the light sources and the position where the FOV of the at least one camera 121 is offset to the at least one camera 121 is L2. L1≤10mm, L1+L2≥30mm.

If the light sources have uncrossed light rays within the distance L1, there may be a dark area in a middle of the image captured within the viewing distance. If the light sources are unable to cover the FOV of the at least one camera 121 beyond the distance L1+L2, there may be a dark area at the edge of the image. Therefore, by making a positional relationship between the at least one camera 121 and the light sources satisfy the above formulas, it is possible to ensure that the light covers the field of view of the at least one camera 121, enhance the lighting effect of the light source 132 on the at least one camera 121, and minimize shadows, light spots or edges.

In some embodiments, in the direction from the imaging side of the at least one camera 121 to the object side of the at least one camera 121, the light sources are lower than the light incident surface of the at least one camera 121. That is, in the direction from the imaging side of the at least one camera 121 to the object side of the at least one camera 121, the light sources are first observed and then the light incident surface of the at least one camera 121 is observed. Further, in the direction from the object side of the at least one camera 121 to the imaging side of the at least one camera 121, the light sources are lower than the light exit surface of the at least one camera 121. That is, in the direction from the imaging side of the at least one camera 121 to the object side of the at least one camera 121, the light source is observed, then the light-emitting surface of the at least one camera 121 is observed.

In other words, the light sources do not protrude from the light incident surface of the at least one camera 121 or the light-emitting surface of the at least one camera 121 in the direction of the optical axis of the at least one camera 121. Such a configuration not only avoids the light sources from blocking the viewing angle of the at least one camera 121, but also helps the illumination range of the light sources to cover the shooting range of the at least one camera 121, so as to avoid the generation of the dark spots and the dark edges in the middle or at the edge of the image.

In some embodiments, in the first form, the camera module 12 moves along the third path. In the second form, the camera module 12 moves along the fourth path. A plane where the third path is located intersects with a plane where the fourth path is located.

The third path is a movement trajectory of the at least one camera 121 outside the oral cavity when the user holds the oral imaging device 1000 and moves the oral imaging device 1000 to shoot the outer surfaces of the teeth. The fourth path is a movement trajectory of the at least one camera 121 inside the oral cavity when the user holds the oral imaging device 1000 and moves the oral imaging device 1000 to shoot the inner surfaces of the teeth. Specifically, the plane where the third path is located is roughly parallel to the outer surfaces of the teeth. Under the third path, the at least one camera 121 moves along the third path outside the oral cavity to capture the image of the outer surfaces of the teeth, which is intuitive and easy to operate. Since the at least one camera 121/the third path is located outside the oral cavity, the discomfort of the patient is reduced, and the risk of infection or injury caused by direct contact with the interior of the oral cavity is avoided.

For instance, the plane where the fourth path is located is roughly parallel to the occlusal surfaces of the teeth, and the fourth path enables the at least one camera 121 to move flexibly inside the oral cavity without hitting the gums and the teeth, thereby shooting the inner surfaces or the occlusal surfaces of the teeth.

In some embodiments, the plane where the third path is located is perpendicular to the plane where the fourth path is located, and the at least one camera 121 is movable and adjustable in the third path and the fourth path, providing which is flexible for the user to operate. For example, in the third path (located outside the mouth), the at least one camera 121 moves along the horizontal plane or a rough horizontal plane to capture an image of the outer surfaces of the teeth; while in the fourth path (located inside the mouth), the at least one camera 121 moves in the plane perpendicular to the third path to shoot the inner surfaces or the occlusal surfaces of the teeth. The third path and the fourth path do not interfere with each other, so the user is allowed to quickly switch the oral imaging device between the first form and the second form according to specific needs without complex adjustment steps, improving operational efficiency and convenience. In different paths, the at least one camera 121 obtains image data in different dimensions, thereby providing a comprehensive and detailed view, ensuring that the at least one camera 121 may capture images from different angles, and reducing the blind spots or information omissions caused by a single perspective.

In some embodiments, the size of the main body 11 is described as follows.

The length of the main body 11 in the length direction is not less than 25 mm, such as 25 mm, 25.4 mm, 26 mm, 26.7 mm, 27 mm, 27.4 mm, 28 mm, 28.5 mm, 29 mm, 29.7 mm, 30 mm, etc.

If the length of the main body 11 is less than 25 mm, a holding portion of the main body for the user to hold is insufficient, which may cause the oral camera 100 to tilt or even fall during the shooting process, thereby affecting the shooting effect and the user experience. Therefore, the length of the main body 11 in the length direction is not less than 25 mm, so the holding portion of the main body for the user to hold is sufficient while providing sufficient accommodating space for the internal components of the oral camera 100.

In some embodiments, the thickness of the main body 11 in the thickness direction thereof is not less than 15 mm and not greater than 30 mm. That is, the distance between the first side surface 112a and the second side surface 112b is not less than 15 mm and not greater than 30 mm.

If the thickness of the main body 11 is less than 15 mm, the main body 11 is too thin, making it difficult to provide sufficient holding space for the user, thereby affecting the control and shooting effect of the user on the oral camera 100. If the thickness of the main body 11 is greater than 30 mm, the main body 11 is too large, which is inconvenient for the user to carry and use, and affects the user experience.

Therefore, by controlling the thickness of the main body 11 within 15-30 mm. On the one hand, the main body 11 is enabled to have a sufficient holding portion for the user to hold to control and shoot the oral camera 100. On the other hand, the main body 11 provides sufficient accommodating space for the internal components of the oral camera 100. In addition, the main body 11 has a certain thickness, which meets the different holding habits of different users and provides sufficient holding space when the user holds the side walls of the main body 11.

In some embodiments, the size of the oral imaging device 1000 is described in detail as follows.

The mouth opener 200 is connected to the end surface of the main body 11 in the length direction. In the first form, in the direction from the first end 22a to the second end 22b of the mouth opener 200, the distance from the first end 22a to the at least one camera 121 is not greater than 20 mm. If the distance from the first end 22a of the mouth opener 200 to the at least one camera 121 is greater than 20 mm, the overall depth of the mouth opener 200 (i.e., the dimension of the mouth opener in the direction from the first end 22a to the second end 22b of the mouth opener 200) increases, thereby making the size of the mouth opener 200 large and difficult to adapt to a narrow oral space. Further, the distance between the at least one camera 121 and the second end 22b of the mouth opener 200 is not enough. As a result, in the first form, the at least one camera 121 is too close to the outer surfaces of the teeth, the shooting distance of the at least one camera 121 is limited, and a wider shooting range is unable to be effectively obtained, thereby affecting the shooting efficiency and shooting effect.

Therefore, in the direction from the first end 22a to the second end 22b of the mouth opener 200, the distance from the at least one camera 121 to the first end 22a of the mouth opener 200 is not greater than 20 mm, which ensures that the at least one camera 121 is at a certain distance from the second end 22b of the mouth opener 200, which ensures that the at least one camera 121 is at an appropriate distance from the outer surfaces of the teeth, ensures that the at least one camera 121 has a certain imaging distance and the imaging range, and ensures shooting efficiency. The depth of the mouth opener 200 is also controlled, so that the imaging distance and the imaging range of the at least one camera 121 are guaranteed without configuring the depth of the mouth opener 200 to be very large. Thus, the mouth opener 200 is prevented from being designed too large.

The distance from the at least one camera 121 to the second end 22b of the mouth opener 200 is not less than 10 mm. If the distance from the at least one camera 121 to the second end 22b of the mouth opener 200 is less than 10 mm, the overall depth of the mouth opener 200 (i.e., the dimension of the mouth opener 200 in the direction from the first end 22a to the second end 22b of the mouth opener 200) increases, thereby making the mouth opener 200 large and difficult to adapt to the narrower oral space. Further, the distance between the at least one camera 121 and the second end 22b of the mouth opener 200 is not enough. In the first form, the at least one camera 121 is too close to the outer surfaces of the teeth, the shooting distance of the at least one camera 121 is limited, and the wide shooting range is unable to be effectively obtained, thereby affecting the shooting efficiency and the shooting effect.

Therefore, the distance between the at least one camera 121 and the second end 22b of the mouth opener 200 is not less than 10 mm, which ensures that the at least one camera 121 has a certain distance from the second end 22b of the mouth opener 200, ensures that the at least one camera 121 has an appropriate distance from the outer surfaces of the teeth, ensures that the at least one camera 121 has a certain imaging distance and imaging range, and ensures the shooting efficiency. The depth of the mouth opener 200 is also controlled, so that the imaging distance and the imaging range of the at least one camera 121 are guaranteed without configuring the depth of the mouth opener 200 to be very large. Thus, the mouth opener 200 is prevented from being designed too large.

In some embodiments, the size of the oral imaging device 1000 is further described as follows.

The support piece 122 includes the connecting end 122a and the insertion end 122b opposite to the connecting end in the length direction of the main body 11. The end surface of the connecting end 122a of the support piece 122 is connected to the main body 11. The at least one camera 121 is disposed at the insertion end 122b of the support piece 122, and the distance from the at least one camera 121 to the end surface of the connecting end 122a of the support piece 122 is not less than 20 mm. If the distance from the at least one camera 121 to the end surface of the connecting end 122a of the support piece 122 is less than 20 mm, the length of the support piece 122 protruding from the main body 11 is short. In the second form, the at least one camera 121 may not be able to fully insert into the oral cavity, resulting in the at least one camera 121 being unable to obtain the image of the teeth deep in the oral cavity, and the shooting range is limited.

Therefore, the distance from the at least one camera 121 to the end surface of the connecting end 122a of the support piece 122 is not less than 20 mm, which ensures that the support piece 122 protrudes and stretches sufficiently relative to the main body 11. Therefore, the at least one camera 121 is able to be inserted into the oral cavity to obtain a comprehensive and in-depth image of the interior of the oral cavity, such as the image of the inner surfaces and the occlusal surfaces of the molars close to the throat.

In some embodiments, in the length direction of the main body 11, the distance between the end surface of the connecting end 122a of the support piece 122 and the end surface of the insertion end 122b of the support piece 122 is not less than 18 mm.

If the distance between the end surface of the connecting end 122a of the support piece 122 and the end surface of the insertion end 122b of the support piece 122 is less than 18 mm, the length of the support piece 122 protruding from the main body 11 is short, which causes the at least one camera 121 to be unable to fully insert into the oral cavity, thereby making it difficult for the at least one camera 121 to obtain the image of the teeth deep in the oral cavity. Further, the shooting range is limited.

Therefore, the distance between the end surface of the connecting end 122a of the support piece 122 and the end surface of the insertion end 122b of the support piece 122 is greater than 18 mm, which ensures that the support piece 122 protrudes from the main body 11 to have a sufficient length, and the at least one camera 121 is allowed to be inserted into the oral cavity to obtain a comprehensive and in-depth image of the oral cavity, such as the inner surfaces and the occlusal surfaces of the molars close to the throat.

Further, in the length direction of the support piece 122, the distance from the at least one camera 121 to the end surface of the insertion end 122b of the support piece 122 is not greater than 8 mm.

If the distance between the at least one camera 121 and the end surface of the insertion end 122b of the support piece 122 is greater than 8 mm, the at least one camera 121 may not be able to fully insert into the oral cavity, which makes it difficult for the at least one camera 121 to obtain the image of the teeth deep in the oral cavity, limits the shooting range, or causes the support piece 122 to poke the soft tissue in the oral cavity.

Therefore, the distance between the at least one camera 121 and the end surface of the insertion end 122b of the support piece 122 is not greater than 8 mm. On the one hand, the at least one camera 121 is able to be inserted into the oral cavity to obtain the comprehensive and in-depth image of the oral cavity, such as the inner surfaces and the occlusal surfaces of the molars near the throat. On the other hand, it prevents the support piece 122 from being excessively inserted into the oral cavity, thereby reducing interference and potential damage to the soft tissue in the oral cavity, and improving the comfort and safety of the oral camera 100.

In some embodiments, the size of the support piece 122 is further described as follows.

The dimension of the support piece 122 in the length direction of the main body 11 is not less than 30 mm. If the dimension of the support piece 122 in the length direction is less than 30 mm, the length of the support piece 122 protruding from the main body 11 is short. In the second form, the at least one camera 121 is unable to fully insert into the oral cavity, resulting in the at least one camera 121 being unable to obtain the image of the teeth deep in the oral cavity, and resulting in a poor shooting range.

Therefore, the dimension of the support piece 122 in the length direction is greater than 30 mm, which ensures that the support piece 122 protrudes and stretches from the main body 11 to a sufficient length, and the at least one camera 121 is able to be inserted into the oral cavity to obtain the comprehensive and in-depth image of the oral cavity, such as the image of the inner surfaces and the occlusal surfaces of the molars near the throat.

The dimension of the support piece 122 in the width direction (i.e, the width of the support piece 122) is not greater than 20 mm. If the dimension of the support piece 122 in the width direction is greater than 20 mm, the support piece 122 is too large and is difficult to move flexibly in the oral cavity, and the support piece 122 may even collide with the teeth or other oral structures, which affects the shooting effect and user experience.

Therefore, the width of the support piece 122 is not greater than 20 mm. On the one hand, the risk of collision between the support piece 122 and the teeth is reduced. On the other hand, the small width of the support piece 122 makes the movement of the support piece 122 in the oral cavity flexible, thereby improving the comfort and safety of the camera module 12.

The dimension of the support piece 122 in the thickness direction (i.e., the thickness of the support piece) is not greater than 15 mm. If the dimension of the support piece 122 in the thickness direction is greater than 15 mm, the support piece 122 is too large and is difficult to move in the oral cavity, and the support piece 122 may collide with the teeth, which affects the shooting effect.

The dimension of the support piece 122 in the thickness direction is not greater than 15 mm, which reduces the risk of the support piece 122 colliding with the teeth or other oral structures. Further, the small thickness of the support piece 122 makes the movement of the support piece 122 in the oral cavity flexible.

In the length direction perpendicular to the main body 11, the area of the cross section of the support piece 122 is not greater than 300mm2. If the area of the cross section of the support piece 122 is greater than 300 mm², the support piece 122 is too large and is difficult to move in the oral cavity, and the support piece 122 may collide with the teeth, which affects the shooting effect and the user experience.

The area of the cross section of the support piece 122 is not greater than 300 mm², which reduces the risk of collision between the support piece 122 and the teeth, and the small width of the support piece allows the support piece 122 to move flexibly in the oral cavity, thereby improving the comfort and safety of the camera module 12.

In some embodiments, the surface formed by the long sides and the wide sides of the support piece 122 is the largest surface of the support piece 122. The at least one camera 121 is on the same side or the opposite side of the largest surface as the oral camera 100.

Specifically, the long sides of the support piece 122 have the maximum dimension of the support piece 122 in the length direction, and the wide sides of the support piece 122 have the maximum dimension of the support piece 122 in the width direction. The surface formed by the long sides and the wide sides of the support piece 122 is perpendicular to the thickness direction of the support piece 122. The dimension of the support piece 122 in the length direction is the maximum dimension of the support piece 122 in the three directions (the length direction, the width direction, and the thickness direction). The surface formed by the long sides and the wide sides of the support piece 122 is the largest surface of the support piece 122, so the support piece 122 presents a relatively flat shape as a whole. Therefore, in the second form, the support piece 122 is easy to be inserted into the mouth, and after being inserted into the mouth, the possibility of hitting against the teeth or the soft tissues (such as the upper dental arch and the tongue) in the oral cavity is reduced, thereby improving the comfort of the user. Moreover, the largest surface of the support piece 122 is placed on the teeth, which well limits the distance between the at least one camera 121 and the part to be scanned (such as the occlusal surfaces or the inner surfaces of the teeth) in the left and right directions, ensuring that at least one of the captured image and the video is uniform and clear. The at least one camera 121 and the largest surface of the support piece are on the same side or opposite side of the oral camera 100, so that the light-emitting direction (the imaging direction) of the at least one camera 121 is always opposite to the largest surface of the support piece. When the user holds the oral camera 100, the largest surface of the support piece faces the oral cavity of the user, which is ergonomic and has good holding stability.

The technical features of the above embodiments can be combined arbitrarily. In order to make the description concise, possible combinations of the technical features in the above embodiments are not described in detail respectively. However, as long as there is no contradiction in the combination of the technical features, the technical features should be considered to be within the scope of this specification.

In addition, the embodiments described above are only some implementation methods of the present disclosure, and the descriptions thereof are relatively specific and detailed, which should not be understood as limiting the scope of the patent of the present disclosure. It should be pointed out that, for those skilled in the art, variations and improvements can be made without departing from the concept of the present disclosure, and the variations and improvements should fall within the protection scope of the present disclosure. Therefore, the content of this specification should not be understood as limiting the present disclosure, and the protection scope of the present disclosure shall be subject to the attached claims.

What is claimed is:

1. An oral imaging device, comprising:
an oral camera; and
a mouth opener;
wherein the oral camera comprises a main body and a camera module connected to the main body, and the camera module comprises at least one camera;
wherein the mouth opener comprises a first end and a second end opposite to the first end, the mouth opener defines an imaging through hole penetrating through the mouth opener from the first end to the second end, and the second end of the mouth opener is configured to abut against an oral cavity structure of a user;
wherein a position of the mouth opener is variable relative to the at least one camera to enable the oral imaging device to have at least a first form and a second form;
wherein in the first form, the at least one camera faces the imaging through hole and does not exceed the second end of the mouth opener;
wherein in the second form, the at least one camera is exposed outside the imaging through hole and is configured to insert into an oral cavity of the user.

2. The oral imaging device according to claim 1, wherein in the first form, the first end of the mouth opener is connected to the main body or the camera module, the second end of the mouth opener is configured to abut against the oral cavity structure, the at least one camera faces the imaging through hole, and the at least one camera does not exceed the second end of the mouth opener in an axial direction of the imaging through hole.

3. The oral imaging device according to claim 1, wherein in the first form, the at least one camera is configured to shoot outside the oral cavity, and in the second form, the at least one camera is configured to shoot inside the oral cavity; and/or
in the first form, the at least one camera is at least configured to shoot outer surfaces of dental arches of the user, and in the second form, the at least one camera is at least configured to shoot occlusal surfaces and/or inner surfaces of the dental arches.

4. The oral imaging device according to claim 1, wherein at least one of a position of the at least one camera and the position of the mouth opener is variable relative to a position of the main body, so that the oral imaging device is switched between the first form and the second form.

5. The oral imaging device according to claim 4, wherein in the first form, the at least one camera is aligned with the imaging through hole;
in the second form, the mouth opener is connected to the main body, and the at least one camera and the imaging through hole are staggered to expose the at least one camera outside the imaging through hole; or
in the second form, the mouth opener is connected to the main body, and the at least one camera stretches outside the imaging through hole, so that the at least one camera is exposed outside the imaging through hole; or
in the second form, the mouth opener is separated from the main body, and the at least one camera protrudes relative to the main body for inserting into the oral cavity.

6. The oral imaging device according to claim 1, wherein the main body extends in a length direction, the main body comprises a first end and a second end opposite to the first end, the first end and the second end of the main body are disposed in the length direction of the main body, the at least one camera is disposed on an end surface of the first end of the main body, an optical axis of the at least one camera and the length direction of the main body are disposed at an angle, and the mouth opener is detachably sleeved on an outer periphery of the first end of the main body, so that the oral imaging device is enabled to switch between the first form and the second form.

7. The oral imaging device according to claim 1, wherein the imaging through hole defines a cross section in an axial direction thereof, a length of the cross section of the imaging through hole in a first direction is greater than a length of the cross section of the imaging through hole in a second direction, and the first direction is perpendicular to the second direction;
in the first form, the first direction of the imaging through hole is substantially parallel to a length direction of the oral imaging device; and/or
in the first form, a geometric center of the imaging through hole is eccentrically disposed relative to a center of the oral imaging device in the length direction; and/or
in the first form, the at least one camera defines a rectangular field of view (FOV), and long sides of the rectangular FOV are parallel to the first direction.

8. The oral imaging device according to claim 1, wherein in the first form, on a projection plane perpendicular to an axis of the imaging through hole, a geometric center of the imaging through hole does not coincide with a geometric center of the mouth opener, or the geometric center of the imaging through hole does not coincide with a geometric center of the oral imaging device.

9. The oral imaging device according to claim 1, wherein the mouth opener comprises a connecting portion and an opening portion, the connecting portion is connected to the oral camera, two ends of the opening portion are respectively the first end of the mouth opener and the second end of the mouth opener, the first end of the mouth opener is connected to the connecting portion, and the imaging through hole is formed in the opening portion.

10. The oral imaging device according to claim 1, wherein the main body extends in a length direction, the main body comprises a connecting surface in the length direction, and the main body further comprises a first peripheral side surface connected to the connecting surface;
wherein the camera module further comprises a support piece, the support piece is connected to the connection surface of the main body, the support piece extends along the length direction of the main body, and the at least one camera is disposed on the support piece;
wherein the mouth opener comprises a connecting portion and an opening portion, the connecting portion is connected to the connecting surface or the first peripheral side surface of the main body, two ends of the opening portion are respectively the first end of the mouth opener and the second end of the mouth opener, the first end of the mouth opener is connected to the connecting portion, and the imaging through hole is formed in the opening portion.

11. The oral imaging device according to claim 10, wherein when the connecting portion is connected to the connecting surface of the main body, a fitting groove is defined in the connecting portion, the fitting groove is matched with the support piece, the fitting groove extends in the length direction of the main body, and the support piece is plugged into the fitting groove in the length direction of the main body.

12. The oral imaging device according to claim 10, wherein the connecting portion comprises a first surface and a second surface, the first surface of the connecting portion is opposite to the second surface of the connecting portion in a thickness direction of the main body, and the opening portion is disposed on the first surface of the connecting portion;
wherein when the connecting portion is connected to the first peripheral side surface of the main body, a peripheral edge of the connecting portion is bent, so that the connecting portion is of a shell structure;
wherein the connecting portion is sleeved on an outer periphery of the main body, and the second surface of the connecting portion is connected to the first peripheral side surface of the main body.

13. The oral imaging device according to claim 1, wherein the main body includes a connecting component disposed around the at least one camera, and the first end of the mouth opener is detachably connected to the connecting component.

14. The oral imaging device according to claim 1, wherein the mouth opener comprises a first end and a second end, the oral imaging device further comprises an isolating sleeve, the isolating sleeve is detachably sleeved on an outer side and an inner side of the mouth opener from the second end of the mouth opener, and the isolating sleeve is configured to isolate the mouth opener and the oral cavity.

15. The oral imaging device according to claim 1, wherein the camera module further comprises a support piece, the support piece is connected to the main body, the at least one camera is disposed on the support piece, the at least one camera defines a rectangular FOV, and short sides of the rectangular FOV are parallel to a length direction of the support piece.

16. The oral imaging device according to claim 1, wherein the camera module further comprises a support piece, the support piece is connected to the main body, the at least one camera is disposed on the support piece, the support piece comprises at least one limiting portion, the at least one limiting portion is disposed between the at least one camera and the main body, the at least one limiting portion is configured to abut against a positioning dental arch of the dental arches of the user and is configured to limit a position of the at least one camera relative to the positioning dental arch.

17. The oral imaging device according to claim 1, wherein the camera module further comprises a support piece, the support piece is connected to the main body, the at least one camera is disposed on the support piece, the support piece comprises at least one abutting portion, and when the support piece abuts against a first dental arch of the user, the at least one camera remains stable through the at least one abutting portion, the at least one camera is configured to capture at least a first image, and the first image comprises an image of at least a first portion of a second dental arch of the user.

18. The oral imaging device according to claim 17, wherein the at least one abutting portion comprises a first abutting portion and second abutting portions, and the second abutting portions and the at least one camera are disposed on adjacent sides of the support piece;
wherein the support piece is configured to abut against at least one of the first dental arch and the second dental arch through the second abutting portions, when in use, the support piece is placed between the first dental arch and the second dental arch, and the at least one camera is configured to capture at least an image of inner surfaces of upper teeth of the user and an image of inner surfaces of lower teeth of the user.

19. The oral imaging device according to claim 17, wherein the at least one abutting portion comprises a first abutting portion and second abutting portions;
wherein the first abutting portion and the at least one camera are disposed on two opposite sides of the support piece, and when the first abutting portion abuts against the first dental arch, the at least one camera moves along a first path to shoot occlusal surfaces of the second dental arch;
wherein the second abutting portions and the at least one camera are disposed on adjacent sides of the support piece, and when the second abutting portions abut against at least one of the first dental arch and the second dental arch, the at least one camera moves along a second path to shoot at least one of inner surfaces of upper teeth of the user and inner surfaces of lower teeth of the user;
wherein a plane where the first path is located intersects with a plane where the second path is located.

20. The oral imaging device according to claim 1, wherein the camera module further comprises a support piece, the support piece is connected to the main body, and the at least one camera is disposed on the support piece;
wherein in a direction perpendicular to a length direction of the main body, at least part of cross sections of the main body is greater than cross sections of the support piece, and the length direction of the main body and a length direction of the support piece are in a same direction; and/or
a thickness of at least part of the main body is greater than a thickness of the support piece; and/or
the main body is a block structure, and the support piece is a rod structure; and/or
a length-to-height ratio of the main body is less than a length-to-height ratio of the support piece.
